(19) United States
(12) Patent Application Publication
(10) Pub. No.: US 2012/0308486 A1
(43) Pub. Date: Dec. 6, 2012

(54) Fragrance and/or flavoring compositions containing dioxolanes

(75) Inventors: Emilie Singer, Holzminden (DE); Bernd Hölscher, Halle (DE)

(73) Assignee: SYMRISE AG, Holzminden (DE)

(21) Appl. No.: 13/473,053

(22) Filed: May 16, 2012

Related U.S. Application Data
(60) Provisional application No. 61/486,998, filed on May 17, 2011.

(30) Foreign Application Priority Data
May 17, 2011 (EP) 11 166 440.5

(51) Int. Cl. A61K 8/49; C07D 317/12; C07D 317/72; A61Q 17/04; A23L 2/56; A61Q 15/00; A61Q 11/00; C11D 3/50; A23L 1/226; A61Q 13/00; A61Q 19/00

(52) U.S. Cl. 424/49; 512/12; 512/9; 549/430; 549/341; 424/59; 514/772; 424/65; 510/103; 426/536

(57) ABSTRACT

The present invention primarily relates to the use of dioxolanes of the following Formula (I) as fragrance and/or flavoring substances, certain perfume and/or flavoring compositions comprising these dioxolanes and corresponding perfumed and/or flavored items.

The present invention also relates to a method for producing the dioxolanes of Formula (I) and certain new dioxolanes according to Formula (I).

The present invention primarily relates to the use of dioxolanes of Formula (I) defined below as fragrance and/or flavoring substance, certain fragrance and/or flavoring compositions comprising these dioxolanes and correspondingly perfumed and/or flavored items.

The present invention also relates to a method for the producing the dioxolanes of Formula (I) and certain new dioxolanes according to Formula (I).

Because of the general scarcity of natural fragrance components, the changes required to keep up with changing flavor preferences and the ever-increasing need for new fragrances, which on their own or in the form of compositions constitute valuable fragrances or perfumes with interesting scents, there continues also to be a need for compounds with interesting fragrance qualities.

Because of the increasing demand from consumers for new scents the perfume industry has a constant need for new fragrance substances with which to achieve novel effects in perfumes and in this way to create fashion trends.

Despite a whole range of fragrance substances already being available the perfume industry also continues to have a general need for new fragrance substances, in particular those which, in addition to their primary, that is to say odor, characteristics also have positive secondary characteristics, such as for example a higher stability under certain application conditions, a high extension, a high radiant power, good diffusivity (e.g. a good spatial effect), fullness, power and/or naturalness, odor-enhancing characteristics or also improved dermatological characteristics compared with fragrance substances with comparable primary odor characteristics.

The perfume industry thus has a basic need for more fragrance substances that are suitable for the production of fragrance compositions or perfumed items. There is in particular a need for fragrances which through the abovementioned technical characteristics lead to increased use of fragrance compositions and perfume oils.

WO 02/085294 describes various acetals for blocking or reducing the unpleasant odor of certain acids. The acetals disclosed there can, according to WO 02/085294, be used in various compositions, inter alia in the area of body care and in cleaning agents.

DE 25 41 438 describes dioxolane aldehyde fragrance substances, which were obtained there by the hydroformylation of the corresponding dioxolanes. The various dioxolanes disclosed in DE 25 41 438 served there as starting materials for the production of the dioxolane aldehyde fragrance substances. The only dixolane described in DE 25 41 438 with regard to odor is 2-hexyl-4-vinyl-1,3-dioxolane, which according to DE 25 41 438 has a fruity odor of pineapple.

EP 1 992 606 A1 discloses certain alkene acetals as fragrance substances. The fragrance substances disclosed in EP 1 992 606 A1 are derived from 4-pentenals, and have in particular a 2,3,3-trimethyl-4-pentenyl structure element.

So the primary object was to find fragrance and/or flavoring substances having an interesting sensory profile and which are suitable for use as fragrance substances in perfumery or as flavoring substances for flavoring consumable preparations, without themselves imparting a pineapple note in the process. In the context of the present invention, therefore, no fragrance substances are sought which exclusively or predominantly smell of pineapple or impart a pineapple note.

Furthermore these fragrance and/or flavoring substances preferably have positive secondary characteristics, preferably odor-enhancing characteristics and/or have the capacity to have odor and/or taste-enhancing characteristics and/or in combination with other fragrance and/or flavoring substances to round off or to increase their naturalness, freshness, (mouth)fill, (radiant) power and/or radiance and/or increase the diffusivity of a fragrance mixture.

This object was achieved by the use of a compound according to Formula (I)

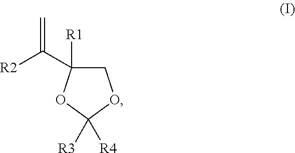

including the stereosiomers, wherein

R1 and R2 in each case and independently of one another is either hydrogen or methyl, preferably hydrogen, and
a) R3 and R4—together with the ketal carbon atom of the dioxolane ring—form a ring with a total of 5, 6, 7 or 8 ring carbon atoms, where the ring optionally contains one or two double bonds and/or optionally 1 to a maximum of 3 oxygen atoms, and wherein the ring is optionally substituted by one or a plurality of branched or unbranched, bridged or unbridged, alkyl groups, alkenyl groups, cycloalkyl groups, cycloalkenyl groups, aryl groups, aryl alkyl groups, alkoxy alkyl groups or alkoxy aryl groups, and wherein R3 and R4 together comprise in total 3 through 30 carbon atoms,

or
b) R3 is either hydrogen or an organic radical with 1 through 15 carbon atoms, wherein the organic radical optionally contains 1 to a maximum of 3 oxygen atoms, preferably R3 is either hydrogen or a saturated or unsaturated, aromatic or aliphatic, branched or unbranched, cyclical or linear, bridged or unbridged structure element with 1 through 15 carbon atoms, wherein the structure element optionally contains 1 to a maximum of 3 oxygen atoms and the structure element is optionally substituted by 1 to a maximum of 3 alkyl- and/or alkenyl groups and/or by one or two hydroxy groups,

and

R4 contains an organic radical with 2 through 15 carbon atoms, wherein the organic radical optionally contains 1 to a maximum of 3 oxygen atoms, preferably R4 is a saturated or unsaturated, aromatic or aliphatic, branched or unbranched, cyclical or linear, bridged or unbridged structure element with 2 through 15 carbon atoms, wherein the structure element optionally contains 1 to a maximum of 3 oxygen atoms and the structure element is optionally substituted by 1 to a maximum of 3 alkyl- and/or alkenyl groups and/or by one or two hydroxy groups

and wherein at least one of the following conditions applies:
i. where R4 is a branched or unbranched alkyl radical with 4 through 15 carbon atoms with optionally one, two or a plurality of double bonds, at position 3 to position 4 of R4 a carbon-carbon single bond is present

or
ii. where R4 is a branched or unbranched alkyl radical with 2 through 15 carbon atoms with optionally one, two or a plurality of double bonds, R3 is not hydrogen,

as a fragrance and/or flavoring substance for imparting, enhancing and/or modifying one, two or a plurality of scents and/or flavors, wherein no scent and/or flavor of pineapple is imparted.

To clarify: condition i. means that between the two carbon atoms at positions 3 and 4 of R4 in particular no carbon-carbon double bond is present.

Preference is for compounds of Formula (I), wherein

R1 and R2 in each case and independently of one another is either hydrogen or methyl, preferably hydrogen, and
a) R3 and R4 together result in a cyclical alkyl system with 5, 6, 7 or 8 carbon atoms, which is optionally substituted by one or a plurality of branched or unbranched alkyl radicals selected from the group consisting of methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl,
3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl, and wherein the alkyl system optionally contains one or two double bonds,

or
b) R3 is either hydrogen or a branched or unbranched alkyl radical with 1 through 15 carbon atoms and optionally one, two or a plurality of double bonds or a cyclical alkyl system with 5, 6 or 7 carbon atoms with optionally one, two or a plurality of double bonds, wherein the alkyl radical is preferably selected from the group consisting of methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl,

and
1. R4 is an aromatic compound, wherein the aromatic compound is optionally substituted by one, two or a plurality of alkyl radicals with 1 through 3 carbon atoms and/or one, two or a plurality of hydroxy radicals and/or one, two or a plurality of alkoxy radicals,

or
2. R4 a mono-, bi- or tricyclic alkyl system, which is optionally substituted by one, two or a

plurality of alkyl radicals, hydroxy radicals or alkoxy radicals, wherein the alkyl radicals are preferably selected from the group consisting of methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl,

or
3. R4 is a branched or unbranched alkyl radical with 2 through 15 carbon atoms with one, two or a plurality of double bonds or a ring system with 5, 6 or 7 carbon atoms with one, two or a plurality of double bonds,

and wherein at least one of the following conditions applies:
i. where R4 is a branched or unbranched alkyl radical with 4 through 15 carbon atoms with optionally one, two or a plurality of double bonds, at position 3 to position 4 of R4 a carbon-carbon single bond is present

or
ii. where R4 is a branched or unbranched alkyl radical with 2 through 15 carbon atoms with optionally one, two or a plurality of double bonds, R3 is a branched or unbranched alkyl radical with 1 through 15 carbon atoms and optionally one, two or a plurality of double bonds.

More preferred compounds of Formula (I) are those in which R1=H and R2=H. More preferred compounds of Formula (I) thus correspond to Formula (I-A):

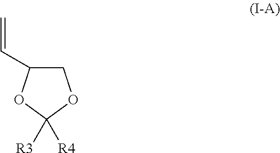

wherein in Formula (I-A) R3 and R4 have the (preferred) meaning given above in relation to Formula (I).

Preference according to the invention is for a use wherein for the compound according to Formula (I) or (I-A) only condition i. applies.

Preference according to the invention is for a use wherein for the compound according to Formula (I) or (I-A) only condition ii. applies.

Preference according to the invention is for a use wherein for the compound according to Formula (I) or (I-A) both conditions i. and ii. apply.

Compounds of Formula (I) or (I-A) preferred according to the invention are the compounds of Formula (II):

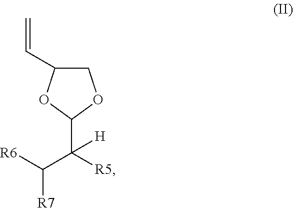

including the stereoisomers,

wherein in Formula (II):

R5 and R6 in each case and independently of one another is either hydrogen or methyl,

R7 is an organic radical with 1 through 10 carbon atoms, wherein the organic radical optionally contains 1 to a maximum of 3 oxygen atoms, preferably R7 is a saturated or unsaturated, aromatic or aliphatic, branched or unbranched, cyclical or linear, bridged or unbridged structure element with 1 through 10 carbon atoms, wherein the structure element optionally contains 1 to a maximum of 3 oxygen atoms and the structure element is optionally substituted by 1 to a maximum of 3 alkyl- and/or alkenyl groups and/or by a hydroxy group, and wherein where R7 is a branched or unbranched alkyl radical with 1 through 10 carbon atoms, at position 1 to position 2 of R7 a carbon-carbon single bond is present.

Preferred compounds according to the invention of Formula (II) are those in which R5 and R6 in each case and independently of one another is either hydrogen or methyl, and

R7 is a branched or unbranched alkyl radical with 1 through 10 carbon atoms, in particular selected from the group consisting of methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl,

or

R7 is a cyloalkyl radical with 5, 6 or 7 carbon atoms, which is optionally substituted by one or a plurality of alkyl radicals, selected from the group consisting of methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl, a hydroxy radical or an alkoxy radical,

and

wherein where R7 is a branched or unbranched alkyl radical with 1 through 10 carbon atoms, at position 1 to position 2 of R7 a carbon-carbon single bond is present.

To clarify: the stipulation regarding R7 means that between the two carbon atoms at positions 1 and 2 of R7 in particular no carbon-carbon double bond is present.

Here the compounds of Formula (I) according to the invention or to be used according to the invention, depending on the substitution pattern, can be present in the form of any mixture of various stereoisomers, in particular as any mixture of diastereomers, here in particular as cis- or trans-isomers, or enantiomers, here in particular as a racemate.

The total number of carbon atoms of the compounds of Formulas (I), (I-A) or (II) is in each case preferably a maximum of 22, preferably a maximum of 18, more preferably a maximum of 16 and particularly preferably a maximum of 14.

The compounds of Formula (I), (I-A) or (II), because of their olfactory characteristics, are eminently suitable for use in fragrance and flavoring substances compositions. The compounds can be used in numerous products in combination with a number of further fragrance or flavoring substances. Particularly advantageously the compound can be combined with other fragrance or flavoring substances in a number of different proportions to form novel fragrance or flavoring compositions.

The compounds of Formula (I) according to the invention or to be used according to the invention are in particular suitable for use as fragrance and/or flavoring substances that can be used in perfumes and/or flavorings. In addition the compounds according to the invention or to be used according to the invention have in particular in alkaline and in oxidizing media a high, quite exceptional stability. In particular because of these characteristics the compounds of Formula (I) are exceptionally well-suited to use as fragrance and/or flavoring substances, and in particular in fact if they are used in perfumed or flavored items or preparations having a pH>7 or an oxidizing effect.

Furthermore the compounds of Formula (I), (I-A) or (II) or fragrance mixtures comprising these compounds have an exceptional diffusivity (spatial effect), making them particularly suitable for incorporation in products for improving ambient air quality (preferably in liquid or gel form, or applied to a solid carrier as well as in pump sprays or aerosol sprays).

A further aspect of the present invention relates to the use of compounds of Formula (I), (I-A) or (II) as enhancers for fragrance or flavoring substances, in particular for fragrance or flavoring substances with flowery (here in particular rosy, indolic), leathery, fruity, fresh, creamy, woody, mossy and/or green notes.

As boosters the compounds of Formula (I), (I-A) or (II) enhance the odor or taste of fragrance or flavoring substances. The so-called boost effect is in perfume oils particularly pronounced in particular in perfume oils with flowery (here in particular rosy, indolic), leathery, fruity, fresh, creamy, woody, mossy and/or green notes.

A corresponding method for modifying and/or enhancing (boosting) an odor or taste with one, a plurality of or all the notes and in particular the flowery notes (here in particular rosy, indolic), leathery, fruity, fresh, creamy, woody, mossy and/or green notes, comprises the following step:
mixing one or a plurality of fragrance or flavoring substances with one, a plurality of or all of the flowery (here in particular rosy, indolic), leathery, fruity, fresh, creamy, woody, mossy and/or green notes, with a quantity of compounds of Formula (I), (I-A) or (II), that is sufficient to sensorially modify and/or enhance the odor and/or taste of the fragrance or flavoring substance(s), that bring about one or a plurality of the flowery (here in particular rosy, indolic), leathery, fruity, fresh, creamy, woody, mossy and/or green notes.

The compounds of Formula (I), (I-A) or (II) according to the invention or to be used according to the invention even in small doses can also enhance the intensity of the fragrance or flavor mixture and round off the overall odor impression of the mixture and can be used to provide a fragrance or flavor composition with more fullness or mouthfill, freshness, (radiant) power, radiance, luster, rounding and/or naturalness.

Where in the context of this text there is a discrepancy between the chemical name and the structure formula shown for the compounds of Formulas (I), (I-A) or (II), the structure formula shown applies.

Particularly preferred compounds of Formula (I) are
2-isobutyl-4-vinyl-[1,3]dioxolane, 2-secbutyl-4-vinyl-[1,3]dioxolane,
2-isopropyl-4-vinyl-[1,3]dioxolane,
2-(3,5-dimethylcyclohex-3-enyl)-4-vinyl-[1,3]dioxolane,
2-(2,4-dimethylcyclohex-3-enyl)-4-vinyl-[1,3]dioxolane,
2-(2,6-dimethylhept-5-enyl)-4-vinyl-[1,3]dioxolane,
2-ethyl-2-(2-methylbutyl)-4-vinyl-[1,3]dioxolane,
2-(2,4,4-trimethylpentyl)-4-vinyl-[1,3]dioxolane,
2-(3,5-dimethylhex-4-enyl)-2-methyl-4-vinyl-[1,3]dioxolane,
2-methyl-2-(4-methylpent-3-enyl)-4-vinyl-[1,3]dioxolane,
8-tertbutyl-2-vinyl-1,4-dioxaspiro-[4.5]-decane,
2-(2,6-dimethylhepta-1,5-dienyl-4-vinyl-[1,3]dioxolane,
2-[2-(4-methylcyclohex-3-enyl)-propyl]-4-vinyl-[1,3]dioxolane,
2-phenyl-4-vinyl-[1,3]dioxolane,
2-cyclohexyl-4-vinyl-[1,3]dioxolane,
2-(2,2,3-trimethylcyclopent-3-enylmethyl)-4-vinyl-[1,3]dioxolane,
2-vinyl-1,4-dioxaspiro[4.5]decane,
7-methyl-2-vinyl-1,4-dioxaspiro[4.5]decane,
2-(2-methylpropenyl)-4-vinyl-[1,3]dioxolane,
2-methyl-2-(3-methylbutyl)-4-vinyl-[1,3]dioxolane.

Quite particularly preferred compounds of Formula (I) are
2-isobutyl-4-vinyl-[1,3]dioxolane,
2-(3,5-dimethylcyclohex-3-enyl)-4-vinyl-[1,3]dioxolane,
2-(2,4-dimethylcyclohex-3-enyl)-4-vinyl-[1,3]dioxolane,
2-(2,6-dimethylhept-5-enyl)-4-vinyl-[1,3]dioxolane,
2-vinyl-1,4-dioxaspiro[4.5]decane,
7-methyl-2-vinyl-1,4-dioxaspiro[4.5]decane,
2-(2-methylpropenyl)-4-vinyl-[1,3]dioxolane, and
2-methyl-2-(3-methylbutyl)-4-vinyl-[1,3]dioxolane.

The most preferred compound of Formula (I) is 2-isobutyl-4-vinyl-[1,3]dioxolane, since this compound has to date had what is probably a unique olfactory profile and on top of this demonstrates the effects described in the context of this invention to a particularly marked extent.

The compounds of Formula (I), (I-A) and (II) according to the invention or to be used according to the invention, (I-A) and (II) can—depending on the specific meaning of the radicals R1 through R4 or R5 through R7—be present in as cis- or trans-isomers. Even though in our own investigations in connection with the present invention the respective cis- or trans-isomers have proved to have somewhat different smells, with regard to the sensory characteristics it has generally not proved detrimental to use isomer mixtures of compounds of Formula (I), (I-A) and (II).

During production the compounds of Formula (I), (I-A) or (II) frequently occur as a mixture of various stereoisomers. For this reason also mixtures of compounds of Formula (I), (I-A) or (II) according to the invention or to be used according to the invention are generally preferred in connection with the present invention.

Where a compound of Formula (I), (I-A) or (II) is used as a mixture of its cis- and trans-isomers, the mass ratio of cis:trans-isomers is preferably in the range 4:1 through 1:4, preferably in the range 3:1 through 1:3, more preferably in the range 2:1 through 1:2, in particular preferably in the range 3:2 through 2:3.

Thus it has been discovered, for example, that the cis-isomer and the trans-isomer of 2-isobutyl-4-vinyl-[1,3]dioxolane have a somewhat different odor from one another. Whereas cis-2-isobutyl-4-vinyl-[1,3]dioxolane has a natural odor of olives, in particular of green olives, coupled with rosy aspects, trans-2-isobutyl-4-vinyl-[1,3]dioxolane on the other hand has an odor of olives, in particular green olives, and garlic, coupled with a fatty note.

Cis-2-isobutyl-4-vinyl-[1,3]dioxolane is in particular suitable as a fragrance m. Trans-2-isobutyl-4-vinyl-[1,3]dioxolane, because of its fatty note, is in particular suited as a fragrance substance.

The following table shows the sensorial, in particular olfactory, descriptions of particularly preferred compounds of Formula (I) to be used according to the invention.

| Number | Compound | Structure | Sensorial description |
|---|---|---|---|
| 1 | 2-isobutyl-4-vinyl- [1,3]dioxolane | 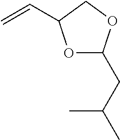 | Strong, green olives, natural |
| 2 | 2-secbutyl-4-vinyl- [1,3]dioxolane | 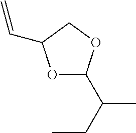 | Fruity |
| 3 | 2-Isopropyl-4-vinyl- [1,3]dioxolane | 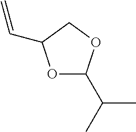 | Metallic, green |
| 4 | 2-(2,4- dimethylcyclohex-3- enyl)-4-vinyl- [1,3]dioxolane | 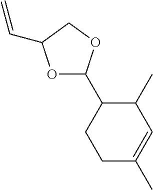 | Fruity, flowery, rose |
| 5 | 2-(2,6- dimethylhept-5- enyl)-4-vinyl- [1,3]dioxolane | 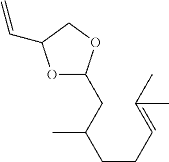 | Fresh, myrrh, fruity, pear, mango, flowery, green |
| 6 | 2-ethyl-2-(2- methylbutyl)-4- vinyl-[1,3]dioxolane | 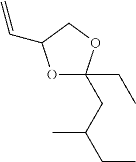 | Fruity, grapefruit |
| 7 | 2-(2,4,4- trimethylpentyl)-4- vinyl-[1,3]dioxolane | 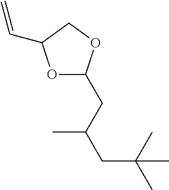 | Watery, fruity, melon |
| 8 | 2-(3,5-dimethylhex- 4-enyl)-2-methyl-4- vinyl-[1,3]dioxolane | 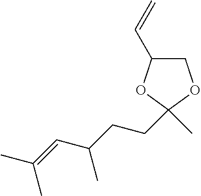 | Fruity, green |
| 9 | 2-methyl-2-(4- methylpent-3-enyl)- 4-vinyl- [1,3]dioxolane | 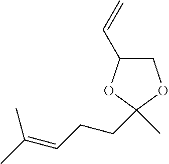 | Green, flowery, aromatic, lemon, linalool |
| 10 | 8-tertbutyl-2-vinyl- 1,4-dioxaspiro- [4.5]-decane | 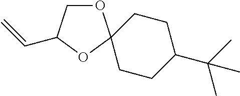 | Fruity, melon |
| 11 | 2-(2,6- dimethylhepta-1,5- dienyl-4-vinyl- [1,3]dioxolane | 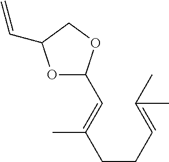 | Fresh, lemon, watery |
| 12 | 2-[2-(4- methylcyclohex-3- enyl)-propyl]-4- vinyl-[1,3]dioxolane | 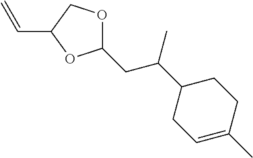 | Fruity, Mirabelle |
| 13 | 2-phenyl-4-vinyl- [1,3]dioxolane | 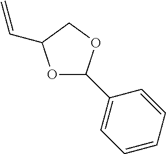 | Herby, spearmint, phenolic, mushroomy, metallic |
| 14 | 2-cyclohexyl-4- vinyl-[1,3]dioxolane | 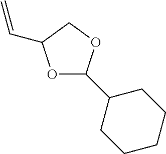 | Fruity, chamomile, apple, aromatic |
| 15 | 2-(2,2,3- trimethylcyclopent- 3-enylmethyl)-4- vinyl-[1,3]dioxolane | 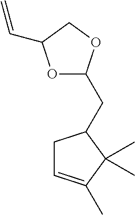 | Fruity, apple, flowers, carbinol |
| 16 | 2-Vinyl-1,4- dioxaspiro[4.5] decane | 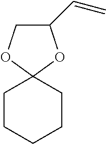 | Fruity, lemon |
| 17 | 7-methyl-2-vinyl- 1,4- dioxaspiro[4.5] decane | 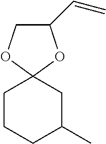 | Fresh, citrus fruits, lemon |
| 18 | 2-(2- methylpropenyl)-4- vinyl-[1,3]dioxolane | 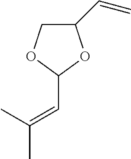 | Herby, fatty |
| 19 | 2-methyl-2-(3- methylbutyl)-4- vinyl-[1,3]dioxolane | 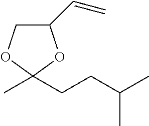 | Fresh, grapefruit |

Preference according to the invention is for the use of a compound of Formula (I), (I-A) or (II), preferably in one of the configurations referred to as preferred or particularly preferred, for imparting, enhancing and/or modifying one, two, three or a plurality of the scents and/or flavors flowery, fatty, green olives, leathery, fruity, green, flowers, rose, myrrh, pear, mango, grapefruit, melon, lemon, linalool, Mirabelle, spearmint, phenolic, mushroomy, metallic, camomile, apple, carbinol, aromatic, watery, fruity, herby.

Accordingly, the invention also relates to a method for imparting, modifying and/or enhancing an odor or taste without imparting the odor or taste of the pineapple note, wherein a sensorially effective quantity of
a fragrance and/or flavoring composition according to the invention (as defined in the following), preferably in one of the configurations referred to as preferred or particularly preferred,

or
a compound according to the invention or to be used according to the invention of Formula (I), (I-A) or (II), preferably in one of the configurations referred to as preferred or particularly preferred,

is brought into contact or mixed with a product,

in particular for imparting, modifying and/or enhancing one, two or a plurality of the scents and/or flavors flowery, fatty, green olives, leathery, fruity, green, flowers, rose, myrrh, pear, mango, grapefruit, melon, lemon, linalool, Mirabelle, spearmint, phenolic, mushroomy, metallic, camomile, apple, carbinol, aromatic, watery, fruity, herby.

The present invention also relates to novel compounds of Formula (I)

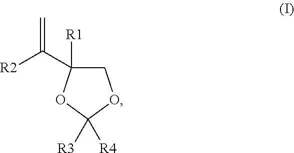

including the stereoisomers,

wherein

R1 and R2 in each case and independently of one another is either hydrogen or methyl, preferably hydrogen,

and
a) R3 and R4 together result in a cyclical alkyl system with 5, 6, 7 or 8 carbon atoms, which is optionally substituted by one or a plurality of branched or unbranched alkyl radicals selected from the group consisting of methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl, and wherein the alkyl system optionally contains one or two double bonds,

or
b) R3 is either hydrogen or a branched or unbranched alkyl radical with 1 through 15 carbon atoms and optionally one, two or a plurality of double bonds or a cyclical alkyl system with 5, 6 or 7 carbon atoms with optionally one, two or a plurality of double bonds, wherein the alkyl radical is preferably selected from the group consisting of methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl,

and
1. R4 is an aromatic compound, wherein the aromatic compound is optionally substituted by one, two or a plurality of alkyl radicals with 1 through 3 carbon atoms and/or one, two or a plurality of hydroxy radicals and/or one, two or a plurality of alkoxy radicals,

or
2. R4 is a mono-, bi- or tricyclic alkyl system, which is optionally substituted by one, two or a plurality of alkyl radicals, hydroxy radicals or alkoxy radicals, wherein the alkyl radicals are in particular selected from the group consisting of methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl,

or
3. R4 is a branched or unbranched alkyl radical with 2 through 15 carbon atoms with optionally one, two or a plurality of double bonds or a ring system with 5, 6 or 7 carbon atoms with optionally one, two or a plurality of double bonds,

and wherein at least one of the following conditions applies:
i. where R4 is a branched or unbranched alkyl radical with 4 through 15 carbon atoms with optionally one, two or a plurality of double bonds, at position 3 to position 4 of R4 a carbon-carbon single bond is present

or
ii. where R4 is a branched or unbranched alkyl radical with 2 through 15 carbon atoms with optionally one, two or a plurality of double bonds, R3 is a branched or unbranched alkyl radical with 1 through 15 carbon atoms and optionally one, two or a plurality of double bonds,

excluding the following compounds:

2-ethyl-4-vinyl-[1,3]dioxolane2-propyl-4-vinyl-[1,3]dioxolane, 2-isopropyl-4-vinyl-[1,3]dioxolane, 2-butyl-4-vinyl-[1,3]dioxolane, 2-isobutyl-4-vinyl-[1,3]dioxolane, 2-pentyl-4-vinyl-[1,3]dioxolane, 2-hexyl-4-vinyl-[1,3]dioxolane, 2-heptyl-4-vinyl-[1,3]dioxolane, 2-cyclohexyl-4-vinyl-[1,3]dioxolane, 2-phenyl-4-vinyl-[1,3]dioxolane, 2-o-tolyl-4-vinyl-[1,3]dioxolane, 2-m-tolyl-4-vinyl-[1,3]dioxolane, 2-p-tolyl-4-vinyl-[1,3]dioxolane, 2-(2-methoxy-phenyl)-4-vinyl-[1,3]dioxolane, 2-(3-methoxy-phenyl)-4-vinyl-[1,3]dioxolane, 2-(4-methoxy-phenyl)-4-vinyl-[1,3]dioxolane, 5-(4-vinyl-[1,3]dioxolane-2-yl)-benzo[1,3]dioxol, 2-furan-3-yl-4-vinyl-[1,3]dioxolane, 2-ethyl-2-methyl-4-vinyl-[1,3]dioxolane, 2-methyl-2-propyl-4-vinyl-[1,3]dioxolane, 2-butyl-2-methyl-4-vinyl-[1,3]dioxolane, 2-isopropyl-2-methyl-4-vinyl-[1,3]dioxolane, 2-methyl-2-(3-methylbutyl)-4-vinyl-[1,3]dioxolane, 2-methyl-2-(4-methyl-pentyl)-4-vinyl-[1,3]dioxolane, 2,2-diethyl-4-vinyl-[1,3]dioxolane, 2,2-dipropyl-4-vinyl-[1,3]dioxolane, 2,2-diisopropyl-4-vinyl-[1,3]dioxolane, 2-methyl-2-pentyl-4-vinyl-[1,3]dioxolane, 2-hexyl-2-methyl-4-vinyl-[1,3]dioxolane, 2-methyl-2-octyl-4-vinyl-[1,3]dioxolane, 2-methyl-2-nonyl-4-vinyl-[1,3]dioxolane, 2-methyl-2-phenyl-4-vinyl-[1,3]dioxolane, 2-cyclohexyl-2-methyl-4-vinyl-[1,3]dioxolane, 2-cyclopentyl-4-vinyl-[1,3]dioxolane, 2-cycloheptyl-4-vinyl-[1,3]dioxolane, 2,2-pentamethylen-4-vinyl-[1,3]-dioxolane, 2-methyl-2-isopropyl-4-vinyl-[1,3]-dioxolane, 2,2-hexamethylen-4-vinyl-[1,3]-dioxolane, 2-but-3-enyl-2-methyl-4-vinyl-[1,3]dioxolane, (S)-2,2-diethyl-4-vinyl-[1,3]dioxolane, (R)-2-vinyl-1,4-dioxa-spiro[4.5]decane, (S)-2-vinyl-1,4-dioxa-spiro[4.5]decane, 2-((e)-propenyl)-4-vinyl-[1,3]dioxolane, 2-(4-vinyl-[1,3]dioxolane-2-yl)-phenol, 2,4-divinyl-[1,3]dioxolane, 2-tert-butyl-2-methyl-4-vinyl-[1,3]dioxolane, 2-butyl-2-ethyl-4-vinyl-[1,3]dioxolane, 2-(2-methyl-cyclohexyl)-4-vinyl-[1,3]dioxolane, 2-(4-methyl-cyclohexyl)-4-vinyl-[1,3]dioxolane, (S)-2-methyl-2-vinyl-1,4-dioxa-spiro[4.5]decane, 2-methyl-2-vinyl-1,4-dioxa-spiro[4.5]decane, (R)-2-(4-methoxy-phenyl)-4-methyl-4-vinyl-[1,3]dioxolane, 4-isopropenyl-2-p-tolyl-[1,3]dioxolane, 4-isopropenyl-2-phenyl-[1,3]dioxolane, (2S,4S)-4-methyl-2-((R)-1-methyl-allyl)-4-vinyl-[1,3]dioxolane, (2R,4R)-4-methyl-2-((R)-1-methyl-allyl)-4-vinyl-[1,3]dioxolane, (2S,4R)-4-methyl-2-((R)-1-methyl-allyl)-4-vinyl-[1,3]dioxolane, (2R,4S)-4-methyl-2-((R)-1-methyl-allyl)-4-vinyl-[1,3]dioxolane, 2-ethyl-2,4-dimethyl-4-vinyl-[1,3]dioxolane, 4-methyl-2-(1-phenyl-ethyl)-4-vinyl-[1,3]dioxolane and 2-isobutyl-2-methyl-4-vinyl-[1,3]dioxolane.

Preference according to the invention is for a compound wherein for the compound according to Formula (I) only condition i. applies.

Preference according to the invention is for a compound, wherein for the compounds according to Formula (I) only condition ii. applies.

Preference according to the invention is for a compound, wherein for the compound according to Formula (I) both conditions i. and ii. apply.

A particularly preferred compound according to the invention is selected from the group consisting of:

2-isobutyl-4-vinyl-[1,3]dioxolane, 2-secbutyl-4-vinyl-[1,3]dioxolane, 2-isopropyl-4-vinyl-[1,3]dioxolane, 2-(3,5-dimethylcyclohex-3-enyl)-4-vinyl-[1,3]dioxolane, 2-(2,4-dimethylcyclohex-3-enyl)-4-vinyl-[1,3]dioxolane, 2-(2,6-dimethylhept-5-enyl)-4-vinyl-[1,3]dioxolane, 2-ethyl-2-(2-methylbutyl)-4-vinyl-[1,3]dioxolane, 2-(2,4,4-trimethylpentyl)-4-vinyl-[1,3]dioxolane, 2-(3,5-dimethylhex-4-enyl)-2-methyl-4-vinyl-[1,3]dioxolane, 2-methyl-2-(4-methylpent-3-enyl)-4-vinyl-[1,3]dioxolane, 8-tertbutyl-2-vinyl-1,4-dioxaspiro-[4.5]-decane, 2-(2,6-dimethylhepta-1,5-dienyl-4-vinyl-[1,3]dioxolane, 2-[2-(4-methylcyclohex-3-enyl)-propyl]-4-vinyl-[1,3]dioxolane, 2-phenyl-4-vinyl-[1,3]dioxolane, 2-cyclohexyl-4-vinyl-[1,3]dioxolane, 2-(2,2,3-trimethylcyclopent-3-enylmethyl)-4-vinyl-[1,3]dioxolane, 2-vinyl-1,4-dioxaspiro[4.5]decane, 7-methyl-2-vinyl-1,4-dioxaspiro[4.5]decane, 2-(2-methylpropenyl)-4-vinyl-[1,3]dioxolane, and 2-methyl-2-(3-methylbutyl)-4-vinyl-[1,3]dioxolane,

and particularly preferably selected from the group consisting of;

2-isobutyl-4-vinyl-[1,3]dioxolane, 2-(3,5-dimethylcyclohex-3-enyl)-4-vinyl-[1,3]dioxolane, 2-(2,4-dimethylcyclohex-3-enyl)-4-vinyl-[1,3]dioxolane, 2-(2,6-dimethylhept-5-enyl)-4-vinyl-[1,3]dioxolane, 2-vinyl-1,4-dioxaspiro[4.5]decane, 7-methyl-2-vinyl-1,4-dioxaspiro[4.5]decane, 2-(2-methylpropenyl)-4-vinyl-[1,3]dioxolane, and 2-methyl-2-(3-methylbutyl)-4-vinyl-[1,3]dioxolane.

The compounds of Formula (I) can by way of example be obtained by reacting 1-butene-3,4-diol and the corresponding aldehydes (if just one of the two groups R3 or R4=H) or ketones (if R3 and R4 do not represent H), as shown by the following aldehydes example of isovaleraldehyde:

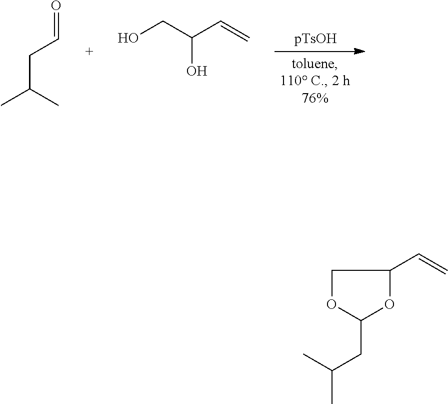

If by way of example isovaleraldehyde in toluene in the presence of p-TsOH (para-toluene sulfonic acid) is reacted with 1-butene-3,4-diol at 110° C., after 2 hours the corresponding dioxolane is obtained with a 76% yield.

1-butene-3,4-diol is not currently commercially available in large quantities, however.

1-butene-3,4-diol can by way of example be produced from 2-butene-1,4-diol, as described in DE 3334589:

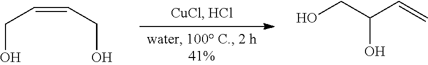

Because of the low overall yield of dioxolanes in the context of the present invention a new synthesis pathway has been developed in order to arrive at compounds of Formula (I).

The following reaction schemess explains by way of example the reaction of isovaleraldehyde with 2-butene-1,4-diol which is in good commercial supply in a method according to the invention:

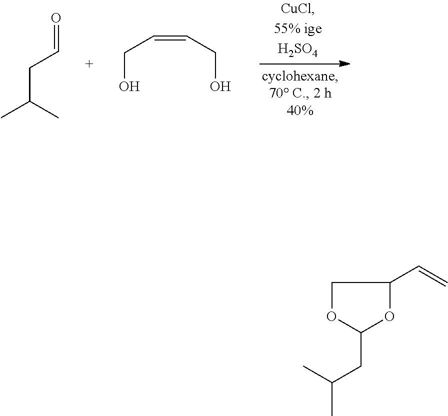

With the method according to the invention the compounds of Formula (I) can be obtained in a single step and in a one-pot reaction using 2-butene-1,4-diol.

In a further aspect, therefore, the invention relates to a method for producing a compound of Formula (I) comprising the following step:

Reaction of a compound of Formula (III) with a compound of Formula (IV) to form the compound of Formula (I) according to the scheme

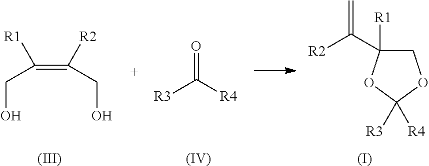

wherein the radicals R1, R2, R3 and R4 of Formulas (III) and (IV) are defined similarly to the corresponding radicals for Formula (I).

Here the radicals R1, R2, R3 and R4 preferably have the meaning indicated above in each case as preferred or particularly preferred.

As a result the compounds of Formula (I) correspond to the compounds of the following structure formula, in which compared with Formula (I) the radicals R1 and R2 are reversed in terms of their positions

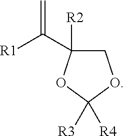

The compounds of Formula (I), (I-A) and (II) indicated above as preferred or particularly preferred in terms of their use apply accordingly to the production method.

The method according to the invention is preferably characterized in that the reaction takes place using copper(I)chloride, preferably with the addition of copper(I)chloride in aqueous sulfuric acid, preferably 55% sulfuric acid, and cyclohexane.

A preferred method according to the invention comprises the following step:

Heating of a mixture of Formula (III), a compound of Formula (IV) and a catalytically effective quantity of copper(I)chloride, preferably in a 2-phase system, preferably in a 2-phase system of aqueous sulfuric acid, particularly preferably 55% sulfuric acid, and cyclohexane.

In a method preferred according to the invention 0.8 through 1.2 molar equivalents of a compound of Formula (III), 0.8 through 1.2 molar equivalents of a compound of Formula (IV) and 0.02 through 0.07 molar equivalents of copper(I)chloride are used.

The method according to the invention is preferably carried out in such a way that the reaction time is 60 through 240 minutes, preferably 80 through 160 minutes, more preferably 100 through 140 minutes, particularly preferably 110 through 130 minutes.

The method according to the invention is preferably carried out at a reaction temperature in the range 40 through 120° C., preferably in the range 50 through 90° C., more preferably in the range 60 through 75° C., in particular preferably in the range 65 through 72° C. The method according to the invention is preferably carried out at ambient pressure, i.e. at a pressure of approximately 1013 mbar.

A method particularly preferred according to the invention is carried out at a reaction temperature in the range 60 through 75° C. for a period of two hours.

Here the following process specification can be used for production of the compounds of Formula (I): a mixture of the diol of Formula (III) (1.00 equiv.), the aldehyde or ketone of Formula (IV) (1.00 equiv.) and copper(I)chloride (0.04 equiv.) is heated in 55% sulfuric acid (44 g/mol) and cyclohexane (22 g/mol) at 70° C. for 2 hours. Following cooling and phase separation the organic phase is washed with water and the aqueous phase is separated again. Following removal of the solvent the raw product can be further cleaned, preferably by distillation over sodium carbonate.

Preference is for a method according to the invention, wherein for the compound of Formula (I) and the compound of Formula (IV) in each case at least one of the following conditions applies:
i. where R4 is a branched or unbranched alkyl radical with 4 through 15 carbon atoms with optionally one, two or a plurality of double bonds, at position 3 to position 4 of R4 a carbon-carbon single bond is present

or
ii. where R4 is a branched or unbranched alkyl radical with 2 through 15 carbon atoms with optionally one, two or a plurality of double bonds, R3 is not hydrogen, and preferably R3 is a branched or unbranched alkyl radical with 1 through 15 carbon atoms and optionally one, two or a plurality of double bonds.

A method preferred according to the invention is a method wherein for the compound according to Formula (I) and the compound of Formula (IV) in each case only the above-mentioned condition i. applies.

A method preferred according to the invention is a method wherein for the compound according to Formula (I) and the compound of Formula (IV) in each case only the above-mentioned condition ii. applies.

A method preferred according to the invention is a method wherein for the compound according to Formula (I) and the compound of Formula (IV) in each case both the above-mentioned conditions i. and ii. apply.

A method according to the invention is particularly suited to the production of the following compounds according to the invention or to be used according to the invention:

2-isobutyl-4-vinyl-[1,3]dioxolane, 2-secbutyl-4-vinyl-[1,3]dioxolane, 2-isopropyl-4-vinyl-[1,3]dioxolane, 2-(3,5-dimethylcyclohex-3-enyl)-4-vinyl-[1,3]dioxolane, 2-(2,4-dimethylcyclohex-3-enyl)-4-vinyl-[1,3]dioxolane, 2-(2,6-dimethylhept-5-enyl)-4-vinyl-[1,3]dioxolane, 2-ethyl-2-(2-methylbutyl)-4-vinyl-[1,3]dioxolane, 2-(2,4,4-trimethylpentyl)-4-vinyl-[1,3]dioxolane, 2-(3,5-dimethylhex-4-enyl)-2-methyl-4-vinyl-[1,3]dioxolane, 2-methyl-2-(4-methylpent-3-enyl)-4-vinyl-[1,3]dioxolane, 8-tertbutyl-2-vinyl-1,4-dioxaspiro-[4.5]-decane, 2-(2,6-dimethylhepta-1,5-dienyl-4-vinyl-[1,3]dioxolane, 2-[2-(4-methylcyclohex-3-enyl)-propyl]-4-vinyl-[1,3]dioxolane, 2-phenyl-4-vinyl-[1,3]dioxolane, 2-cyclohexyl-4-vinyl-[1,3]dioxolane, 2-(2,2,3-trimethylcyclopent-3-enylmethyl)-4-vinyl-[1,3]dioxolane, 2-vinyl-1,4-dioxaspiro[4.5]decane, 7-methyl-2-vinyl-1,4-dioxaspiro[4.5]decane, 2-(2-methylpropenyl)-4-vinyl-[1,3]dioxolane, and 2-methyl-2-(3-methylbutyl)-4-vinyl-[1,3]dioxolane.

A method according to the invention is particularly suitable for producing the following compounds that are preferred or particularly preferred for use according the invention:

2-isobutyl-4-vinyl-[1,3]dioxolane, 2-(3,5-dimethylcyclohex-3-enyl)-4-vinyl-[1,3]dioxolane, 2-(2,4-dimethylcyclohex-3-enyl)-4-vinyl-[1,3]dioxolane, 2-(2,6-dimethylhept-5-enyl)-4-vinyl-[1,3]dioxolane, 2-vinyl-1,4-dioxaspiro[4.5]decane, 7-methyl-2-vinyl-1,4-dioxaspiro[4.5]decane, 2-(2-methylpropenyl)-4-vinyl-[1,3]dioxolane, and 2-methyl-2-(3-methylbutyl)-4-vinyl-[1,3]dioxolane.

In a particularly preferred configuration in a method according to the invention the compound of Formula (III) is 2-butene-1,4-diol and the compound of Formula (IV) isovaleraldehyde.

The invention also relates to a fragrance and/or flavoring composition comprising one, two, three or a plurality of compounds of Formula (I),

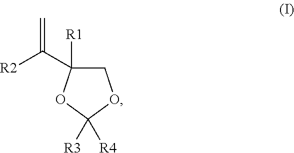

including the stereosiomers, wherein

R1 and R2 in each case and independently of one another is either hydrogen or methyl, preferably hydrogen, and
a) R3 and R4 form a ring with a total of 5, 6, 7 or 8 ring carbon atoms, wherein the ring optionally contains one or two double bonds and/or optionally 1 to a maximum of 3 oxygen atoms, and wherein the ring is optionally substituted by one or a plurality of branched or unbranched, bridged or unbridged, alkyl groups, alkenyl groups, cycloalkyl groups, cycloalkenyl groups, aryl groups, aryl alkyl groups, alkoxy alkyl groups or alkoxy aryl groups, and wherein R3 and R4 together comprise in total 3 through 30 carbon atoms,

or
b) R3 is either hydrogen or an organic radical with 1 through 15 carbon atoms, wherein the organic radical optionally contains 1 to a maximum of 3 oxygen atoms, preferably R3 is either hydrogen or a saturated or unsaturated, aromatic or aliphatic, branched or unbranched, cyclical or linear, bridged or unbridged structure element with 1 through 15 carbon atoms, wherein the structure element optionally contains 1 to a maximum of 3 oxygen atoms and the structure element is optionally substituted by 1 to a maximum of 3 alkyl- and/or alkenyl groups and/or by one or two hydroxy groups,

and

R4 is an organic radical with 2 through 15 carbon atoms, wherein the organic radical optionally contains 1 to a maximum of 3 oxygen atoms, preferably R4 is a saturated or unsaturated, aromatic or aliphatic, branched or unbranched, cyclical or linear, bridged or unbridged structure element with 2 through 15 carbon atoms, wherein the structure element optionally contains 1 to a maximum of 3 oxygen atoms and the structure element is optionally substituted by 1 to a maximum of 3 alkyl- and/or alkenyl groups and/or by one or two hydroxy groups,

and wherein at least one of the following conditions applies:
i. where R4 is a branched or unbranched alkyl radical with 4 through 15 carbon atoms with optionally one, two or a plurality of double bonds, at position 3 to position 4 of R4 a carbon-carbon single bond is present

or
ii. where R4 is a branched or unbranched alkyl radical with 2 through 15 carbon atoms with optionally one, two or a plurality of double bonds, R3 is not hydrogen, and wherein the fragrance and/or flavoring composition contains at least 3, 4, 5, 6, 7, 8 or a plurality of further fragrance and/or flavoring substances, with in each case this not being a matter of a compound of Formula (I),

and wherein the fragrance and/or flavoring substance composition does not contain 2-hexyl-4-vinyl-1,3-dioxolane.

A fragrance and/or flavoring composition preferred according to the invention comprises one, two, three or a plurality of compounds of the above Formula (I), wherein

R1 and R2 in each case and independently of one another is either hydrogen or methyl, in each case preferably hydrogen,

and
a) R3 and R4 together result in a cyclical alkyl system with 5, 6, 7 or 8 carbon atoms, which is optionally substituted by one or a plurality of branched or unbranched alkyl radicals selected from the group consisting of methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl, and wherein the alkyl system optionally contains one or two double bonds,

or
b) R3 is either hydrogen or a branched or unbranched alkyl radical with 1 through 15 carbon atoms and optionally one, two or a plurality of double bonds or a cyclical alkyl system with 5, 6 or 7 carbon atoms with optionally one, two or a plurality of double bonds, wherein the alkyl radical is preferably selected from the group consisting of methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl,

and
1. R4 is an aromatic compound, wherein the aromatic compound is optionally substituted by one, two or a plurality of alkyl radicals with 1 through 3 carbon atoms and/or one, two or a plurality of hydroxy radicals and/or one, two or a plurality of alkoxy radicals,

or
2. R4 is a mono-, bi- or tricyclic alkyl system, that is optionally substituted by one, two or a plurality of alkyl radicals, hydroxy radicals or alkoxy radicals, wherein the alkyl radicals are preferably selected from the group consisting of methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl,

or
3. R4 is a branched or unbranched alkyl radical with 2 through 15 carbon atoms with optionally one, two or a plurality of double bonds or a ring system with 5, 6 or 7 carbon atoms with optionally one, two or a plurality of double bonds,

and wherein the fragrance and/or flavoring composition contains at least 3, 4, 5, 6, 7, 8 or a plurality of further fragrance and/or flavoring substances, with in each case this not being a matter of a compound of Formula (I),

and wherein the fragrance and/or flavoring composition does not contain 2-Hexyl-4-vinyl-1,3-dioxolane.

A fragrance and/or flavoring composition more preferred according to the invention comprises one, two, three or a plurality of compounds of Formula (II),

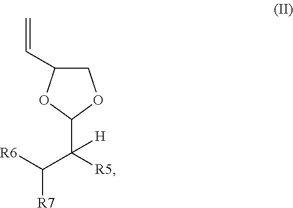

including the stereoisomers, wherein
a) R5 and R6 in each case and independently of one another is either hydrogen or methyl,

and
b) R7 is an organic radical with 1 through 10 carbon atoms, wherein the organic radical optionally contains 1 to a maximum of 3 oxygen atoms, preferably R7 is a saturated or unsaturated, aromatic or aliphatic, branched or unbranched, cyclical or linear, bridged or unbridged structure element with 1 through 10 carbon atoms, wherein the structure element optionally contains 1 to a maximum of 3 oxygen atoms and the structure element is optionally substituted by 1 to a maximum of 3 alkyl- and/or alkenyl groups and/or by a hydroxy group, and

wherein where R7 is a branched or unbranched alkyl radical with 1 through 10 carbon atoms, at position 1 to position 2 of R7 a carbon-carbon single bond is present.

A fragrance and/or flavoring composition preferred according to the invention comprises one, two, three or a plurality of compounds of the above Formula (II), wherein

R5 and R6 in each case and independently of one another is either hydrogen or methyl, and

R7 is a branched or unbranched alkyl radical with 1 through 10 carbon atoms, preferably selected from the group consisting of methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl,

or

R7 is a cyloalkyl radical with 5, 6 or 7 carbon atoms, which is optionally substituted by one or a plurality of alkyl radicals, selected from the group consisting of methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl, a hydroxy radical or an alkoxy radical,

and

wherein where R7 is a branched or unbranched alkyl radical with 1 through 10 carbon atoms, at position 1 to position 2 of R7 a carbon-carbon single bond is present.

A fragrance and/or flavoring composition more preferred according to the invention comprises one, two, three or a plurality of compounds of the above Formula (I), selected from the group consisting of 2-isobutyl-4-vinyl-[1,3]dioxolane, 2-secbutyl-4-vinyl-[1,3]dioxolane, 2-isopropyl-4-vinyl-[1,3]dioxolane, 2-(3,5-dimethylcyclohex-3-enyl)-4-vinyl-[1,3]dioxolane, 2-(2,4-dimethylcyclohex-3-enyl)-4-vinyl-[1,3]dioxolane, 2-(2,6-dimethylhept-5-enyl)-4-vinyl-[1,3]dioxolane, 2-ethyl-2-(2-methylbutyl)-4-vinyl-[1,3]dioxolane, 2-(2,4,4-trimethylpentyl)-4-vinyl-[1,3]dioxolane, 2-(3,5-dimethylhex-4-enyl)-2-methyl-4-vinyl-[1,3]dioxolane, 2-methyl-2-(4-methylpent-3-enyl)-4-vinyl-[1,3]dioxolane, 8-tertbutyl-2-vinyl-1,4-dioxaspiro-[4.5]-decane, 2-(2,6-dimethylhepta-1,5-dienyl-4-vinyl-[1,3]dioxolane, 2-[2-(4-methylcyclohex-3-enyl)-propyl]-4-vinyl-[1,3]dioxolane, 2-phenyl-4-vinyl-[1,3]dioxolane, 2-cyclohexyl-4-vinyl-[1,3]dioxolane, 2-(2,2,3-trimethylcyclopent-3-enylmethyl)-4-vinyl-[1,3]dioxolane, 2-vinyl-1,4-dioxaspiro[4.5]decane, 7-methyl-2-vinyl-1,4-dioxaspiro[4.5]decane, 2-(2-methylpropenyl)-4-vinyl-[1,3]dioxolane, and 2-methyl-2-(3-methylbutyl)-4-vinyl-[1,3]dioxolane.

A fragrance and/or flavoring composition particularly preferred according to the invention comprises one, two, three or a plurality of compounds of the above Formula (I), selected from the group consisting of

2-isobutyl-4-vinyl-[1,3]dioxolane, 2-(3,5-dimethylcyclohex-3-enyl)-4-vinyl-[1,3]dioxolane, 2-(2,4-dimethylcyclohex-3-enyl)-4-vinyl-[1,3]dioxolane, 2-(2,6-dimethylhept-5-enyl)-4-vinyl-[1,3]dioxolane, 2-vinyl-1,4-dioxaspiro[4.5]decane, 7-methyl-2-vinyl-1,4-dioxaspiro[4.5]decane, 2-(2-methylpropenyl)-4-vinyl-[1,3]dioxolane, and 2-methyl-2-(3-methylbutyl)-4-vinyl-[1,3]dioxolane.

In a flavoring composition according to the invention (as defined above) preferably one or a plurality of, particularly preferably two, three, four, five, six, seven, eight, nine, ten or all the further flavorings are selected from the group consisting of:

acetophenone, allyl caproate, alpha-ionone, beta-ionone, anisaldehyde, anisyl acetate, anisyl formate, benzaldehyde, benzothiazole, benzyl acetate, benzyl alcohol, benzyl benzoate, beta-ionone, butyric acid, butyl butyrate, butyl caproate, butylidene phthalide, carvone, camphene, caryophyllene, cineol, cinnamyl acetate, citral, citronellol, citronellal, citronellyl acetate, cyclohexyl acetate, cymol, damascone, decalactone, acetic acid, dihydrocoumarin, dimethyl anthranilate, dimethyl anthranilate, dodecalactone, ethoxyethyl acetate, ethylbutyric acid, ethyl butyrate, ethyl caprinate, ethyl caproate, ethyl crotonate, ethyl furaneol, ethyl guajacol, ethyl isobutyrate, ethyl isovalerate, ethyl lactate, ethyl methyl butyrate, ethyl propionate, eucalyptol, eugenol, ethyl heptylate, 4-(p-hydroxyphenyl)-2-(butanone), gamma-decalactone, geraniol, geranyl acetate, geranyl acetate, grapefruit aldehyde, methyl dihydrojasmonate (Hedion®), heliotropin, 2-heptanone, 3-heptanone, 4-heptanone, trans-2-heptenal, cis-4-heptenal, trans-2-hexenal, cis-3-hexenol, trans-2-hexenoic acid, trans-3-hexenoic acid, cis-2-hexenyl acetate, cis-3-hexenyl acetate, cis-3-hexenyl caproate, trans-2-hexenyl caproate, cis-3-hexenyl formate, cis-2-hexyl acetate, cis-3-hexyl acetate, trans-2-hexyl acetate, cis-3-hexyl formate, para-hydroxy benzyl acetone, isoamyl alcohol, isoamyl isovalerate, isobutyl butyrate, isobutyraldehyde, isoeugenol methyl ether, isopropylmethylthiazole, lauric acid, levulinic acid, linalool, linalool oxide, linalyl acetate, menthol, menthofuran, methyl anthranilate, methylbutanol, methylbutyric acid, 2-methylbutyl acetate, methyl caproate, methyl cinnamate, 5-methyl furfural, 3,2,2-methyl cyclopentenolone, 6,5,2-methyl heptenone, methyl dihydrojasmonate, methyl jasmonate, 2-methyl methyl butyrate, 2-methyl-2-pentenoic acid, methylthiobutyrate, 3,1-methylthiohexanol, 3-methylthiohexyl acetate, nerol, neryl acetate, trans, trans, 2,4-nonadienal, 2,4-nonadienol, 2,6-nonadienol, 2,4-nonadienol, nootkatone, delta-octalactone, gamma-octalactone, 2-octanol, 3-octanol, 1,3-octenol, 1-octyl acetate, 3-octyl acetate, palmitic acid, paraldehyde, phellandrene, pentanedione, phenylethyl acetate, phenylethyl alcohol, phenylethyl alcohol, phenylethyl isovalerate, piperonal, propionaldehyde, propyl butyrate, pulegone, pulegol, sinensal, sulfurol, terpinene, terpineol, terpinolene, 8,3-thiomenthanone, 4,4,2-thiomethyl pentanone, thymol, delta-undecalactone, gamma-undecalactone, valencene, valeric acid, vanillin, acetoin, ethyl vanillin, ethyl vanillin isobutyrate (=3-ethoxy-4-isobutyryloxybenzaldehyde), 2,5-dimethyl-4-hydroxy-3(2H)-furanone and the derivatives thereof (in this case in particular homofuraneol (=2-ethyl-4-hydroxy-5-methyl-3(2H)-furanone), homofuronol (=2-ethyl-5-methyl-4-hydroxy-3(2H)-furanone and 5-ethyl-2-methyl-4-hydroxy-3(2H)-furanone), maltol and maltol derivatives (in this case preferably ethyl maltol), coumarin and coumarin derivatives, gamma-lactones (in this case preferably gamma-undecalactone, gamma-nonalactone, gamma-decalactone), delta-lactones (in this case preferably 4-methyl delta decalactone, massoia lactone, delta decalactone, tuberose lactone), methyl sorbate, divanillin, 4-hydroxy-2(or 5)-ethyl-5(or 2)-methyl-3(2H)furanone, 2-hydroxy-3-methyl-2-cyclopentenone, 3-hydroxy-4,5-dimethyl-2(5H)-furanone, acetic acid isoamyl ester, butyric acid ethyl ester, butyric acid-n-butyl ester, butyric acid isoamyl ester, 3-methylbutyric acid ethyl ester, n-hexanoic acid ethyl ester, n-hexanoic acid allyl ester, n-hexanoic acid-n-butyl ester, n-octanoic acid ethyl ester, ethyl-3-methyl-3-phenyl glycidate, ethyl-2-trans-4-cis-decadienoate, 4-(p-hydroxyphenyl)-2-butanone, 1,1-dimethoxy-2,2,5-trimethyl-4-hexane, 2,6-dimethyl-5-hepten-1-al and phenylacetaldehyde, 2-methyl-3-(methylthio)furan, 2-methyl-3-furanthiol, bis(2-methyl-3-furyl)disulphide, furfuryl mercaptan, methional, 2-acetyl-2-thiazoline, 3-mercapto-2-pentanone, 2,5-dimethyl-3-furanthiol, 2,4,5-trimethylthiazole, 2-acetylthiazole, 2,4-dimethyl-5-ethylthiazole, 2-acetyl-1-pyrroline, 2-methyl-3-ethylpyrazine, 2-ethyl-3,5-dimethylpyrazine, 2-ethyl-3,6-dimethylpyrazine, 2,3-diethyl-5-methylpyrazine, 3-isopropyl-2-methoxypyrazine, 3-isobutyl-2-methoxypyrazine, 2-acetylpyrazine, 2-pentylpyridine, (E,E)-2,4-decadienal, (E,E)-2,4-nonadienal, (E)-2-octenal, (E)-2-nonenal, 2-undecenal, 12-methyltridecanal, 1-penten-3-one, 4-hydroxy-2,5-dimethyl-3(2H)-furanone, guajacol, 3-hydroxy-4,5-dimethyl-2(5H)-furanone, 3-hydroxy-4-methyl-5-ethyl-2(5H)-furanone, cinnamaldehyde, cinnamyl alcohol, methyl salicylate, isopulegol and stereoisomers, enantiomers, enantiomers, diastereomers and possibly cis/trans-isomers thereof.

Examples of fragrance substances, with which the compounds of formula (I) according to the invention or to be used according to the invention can be advantageously combined and which accordingly are preferably contained in a fragrance composition according to the invention (as defined above) can be found, for example, in S. Arctander, Fragrance and Flavor Materials, Vol. I and II, Montclair, N.J., 1969, author's edition or H. Surburg, J. Panten, Common Fragrance and Flavor Materials, 5th. Ed., Wiley-VCH, Weinheim 2006.

In a fragrance composition (as defined above) preferably one or a plurality of, particularly preferably two, three, four, five, six, seven, eight, nine, ten or all the further fragrance substances are selected from the group consisting of:

extracts of natural raw materials such as essential oils, concretes, absolutes, resins, resinoids, balsams and tinctures, such as for example ambergris tincture; amyris oil; angelica seed oil; angelica root oil; anise oil; valerian oil; basil oil; wood moss absolute; bay oil; mugwort oil; benzoin resin; bergamot oil; beeswax absolute; birch tar oil; bitter almond oil; savory oil; bucco leaf oil; cabreuva oil; cade oil; calmus oil; camphor oil; cananga oil; cardamom oil; cascarilla oil; cassia oil; cassia absolute; castoreum absolute; cedar leaf oil; cedarwood oil; cistus oil; citronella oil; lemon oil; copaiva balsam; copaiva balsam oil; coriander oil; costus root oil; cumin oil; cypress oil; davana oil; dill herb oil; dill seed oil; eau de brouts absolute; oakmoss absolute; elemi oil; estragon oil; eucalyptus citriodora oil; eucalyptus oil; fennel oil; spruce needle oil; galbanum oil; galbanum resin; geranium oil; grapefruit oil; guaiac wood oil; gurjun balsam; gurjun balsam oil; helichrysum absolute; helichrysum oil; ginger oil; iris root absolute; iris root oil; jasmine absolute; calamus oil; camomile blue oil; camomile Roman oil; carrot seed oil; cascarilla oil; pine needle oil; spearmint oil; caraway oil; labdanum oil; labdanum absolute; labdanum resin; lavandin absolute; lavandin oil; lavender absolute; lavender oil; lemongrass oil; lavage oil; distilled lime oil; pressed lime oil; linaloe oil; litsea cubeba oil; bayleaf oil; mace oil; marjoram oil; mandarin oil; massoi bark oil; mimosa absolute; musk seed oil; musk tincture; clary sage oil; nutmeg oil; myrrh absolute; myrrh oil; myrtenol; clove leaf oil; clove flower oil; neroli oil; olibanum absolute; olibanum oil; opopanax oil; orange-flower absolute; orange oil; origanum oil; palmarosa oil; patchouli oil; perilla oil; peruvian balsam oil; parsley leaf oil; parsley seed oil; petitgrain oil; peppermint oil; pepper oil; pimenta oil; pine oil; pennyroyal oil; rose absolute; rosewood oil; rose oil; rosemary oil; dalmation sage oil; Spanish sage oil; sandalwood oil; celery seed oil; spike lavender oil; Japanese anise oil; styrax oil; tagetes oil; fir needle oil; teatree oil; turpentine oil; thyme oil; tolu balsam; tonka absolute; tuberose absolute; vanilla extract; violet leaf absolute; verbena oil; vetiver oil; juniper oil; wine lees oil; absinthe oil; wintergreen oil; ylang oil; hyssop oil; civet absolute; cinnamon leaf oil; cinnamon bark oil; and fractions thereof, or ingredients isolated therefrom; individual fragrances from the group of

the hydrocarbons 3-carene; α-pinene; β-pinene; α-terpinene; γ-terpinene; p-cymene; bisabolene; camphene; caryophyllene; cedrene; farnesene; limonene; longifolene; myrcene; ocimene; valencene; (E,Z)-1,3,5-undecatriene; styrene; diphenyl methane;

the aliphatic alcohols such as for example hexanol; octanol; 3-octanol; 2,6-dimethylheptanol; 2-methylheptanol; 2-methyloctanol; (E)-2-hexenol; (E)- and (Z)-3-hexenol; 1-octen-3-ol; mixture of 3,4,5,6,6-pentamethyl-3/4-hepten-2-ol and 3,5,6,6-tetramethyl-4-methyleneheptan-2-ol; (E,Z)-2,6-nonadienol; 3,7-dimethyl-7-methoxyoctan-2-ol; 9-decenol; 10-undecenol; 4-methyl-3-decen-5-ol;

the aliphatic aldehydes and the acetals thereof, which do not correspond to the Formulas (I), (I-A) and (II) such as for example hexanal; heptanal; octanal; nonanal; decanal; undecanal; dodecanal; tridecanal; 2-methyloctanal; 2-methylnonanal; (E)-2-hexenal; (Z)-4-heptenal; 2,6-dimethyl-5-heptenal; 10-undecenal; (E)-4-decenal; 2-dodecenal; 2,6,10-trimethyl-5,9-undecadienal; heptanaldiethylacetal; 1,1-dimethoxy-2,2,5-trimethyl-4-hexene; citronellyloxyacetaldehyde;

the aliphatic ketones and the oximes thereof such as for example 2-heptanone; 2-octanone; 3-octanone; 2-nonanone; 5-methyl-3-heptanone; 5-methyl-3-heptanone oxime; 2,4,4,7-tetramethyl-6-octen-3-one; 6-methyl-5-hepten-2-one;

the aliphatic sulfur-containing compounds such as for example 3-methylthiohexanol; 3-methylthiohexyl acetate; 3-mercaptohexanol; 3-mercaptohexyl acetate; 3-mercaptohexyl butyrate; 3-acetylthiohexyl acetate; 1-menthene-8-thiol;

the aliphatic nitriles such as for example 2-nonenenitrile; 2-tridecenenitrile; 2,12-tridecadienenitrile; 3,7-dimethyl-2,6-octadienenitrile; 3,7-dimethyl-6-octenenitrile;

the aliphatic carboxylic acids and the esters thereof such as for example (E)- and (Z)-3-hexenyl formate; ethyl acetoacetate; isoamyl acetate; hexyl acetate; 3,5,5-trimethylhexyl acetate; 3-methyl-2-butenyl acetate; (E)-2-hexenyl acetate; (E)- and (Z)-3-hexenyl acetate; octyl acetate; 3-octyl acetate; 1-octen-3-yl acetate; ethyl butyrate; butyl butyrate, isoamyl butyrate; hexyl butyrate; (E)- and (Z)-3-hexenyl isobutyrate; hexyl crotonate; ethyl isovalerate; ethyl-2-methylpentanoate; ethyl hexanoate; allyl hexanoate; ethyl heptanoate; allyl heptanoate; ethyl octanoate; ethyl(E,Z)-2,4-decadienoate; methyl-2-octynoate; methyl-2-nonynoate; allyl 2-isoamyl oxyacetate; methyl-3,7-dimethyl-2,6-octadienoate;

the acyclic terpene alcohols such as for example citronellol; geraniol; nerol; linalool; lavandulol; nerolidol; farnesol; tetrahydrolinalool; tetrahydrogeraniol; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2-ol; 3,7-dimethyl-4,6-octadien-3-ol; 3,7-dimethyl-1,5,7-octatrien-3-ol; 2,6-dimethyl-2,5,7-octatrien-1-ol; and the formates, acetates, propionates, isobutyrates, butyrates, isovalerates, pentanoates, hexanoates, crotonates, tiglinates, 3-methyl-2-butenoates thereof;

the acyclic terpene aldehydes and ketones such as for example geranial; neral; cirtonellal; 7-hydroxy-3,7-dimethyloctanal; 7-methoxy-3,7-dimethyloctanal; 2,6,10-trimethyl-9-undecenal; geranylacetone; and the dimethyl and diethyl acetals of geranial, neral, 7-hydroxy-3,7-dimethyloctanal;

the cyclic terpene alcohols such as for example menthol; isopulegol; alpha-terpineol; terpineol-4; menthan-8-ol; menthan-1-ol; menthan-7-ol; borneol; isoborneol; linalool oxide; nopol; cedrol; ambrinol; vetiverol; guajol; and the formates, acetates, propionates, isobutyrates, butyrates, isovalerates, pentanoates, hexanoates, crotonates, tiglinates, 3-methyl-2-butenoates thereof;

the cyclic terpene aldehydes and ketones such as for example menthone; isomenthone; 8-mercaptomenthan-3-one; carvone; camphor; fenchone; alpha-ionone; beta-ionone; alpha-n-methylionone; beta-n-methylionone; alpha-isomethylionone; beta-isomethylionone; alpha-iron; alpha-damascone; beta-damascone; beta-damascenone; delta-damascone; gamma-damascone; 1-(2,4,4-trimethyl-2-cyclo-hexen-1-yl)-2-buten-1-one; 1,3,4,6,7,8a-hexahydro-1,1,5,5-tetramethyl-2H-2,4a-methanona phthalen-8(5H)-one; nootkatone; dihydronootkatone; alpha-sinensal; beta-sinensal; acetylated cedar wood oil (methylcedryl ketone);

the cyclic alcohols such as for example 4-tert-butylcyclohexanol; 3,3,5-trimethylcyclohexanol; 3-isocamphylcyclohexanol; 2,6,9-trimethyl-Z2,Z5, E9-cyclododecatrien-1-ol; 2-isobutyl-4-methyltetrahydro-2H-pyran-4-ol;

the cycloaliphatic alcohols such as for example alpha-3,3-trimethylcyclohexylmethanol; 2-methyl-4-(2,2,3-trimethyl-3-cyclopent-1-yl)butanol; 2-methyl-4-(2,2,3-trimethyl-3-cyclopent-1-yl)-2-buten-1-ol; 2-ethyl-4-(2,2,3-trimethyl-3-cyclopent-1-yl)-2-buten-1-ol; 3-methyl-5-(2,2,3-trimethyl-3-cyclopent-1-yl)-pentan-2-ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl)pentan-3-ol; 1-(2,2,6-trimethylcyclohexyl)-hexan-3-ol;

the cyclic and cycloaliphatic ethers, such as, for example, cineol; cedryl methyl ether; cyclododecyl methyl ether; (ethoxymethoxy)cyclododecane; alpha-cedrene epoxide; 3a,6,6,9a-tetramethyldodecahydronaphtho[2,1-b]furan; 3a-ethyl-6,6,9a-trimethyldodecahydronaphtho[2,1-b]furan; 1,5,9-trimethyl-13-oxabicyclo[10.1.0]trideca-4,8-diene; rose oxide; 2-(2,4-dimethyl-3-cyclohexen-1-yl)-5-methyl-5-(1-methylpropy I)-1,3-dioxane;

the cyclic and macrocyclic ketones, such as, for example, 4-tert.-butylcyclohexanone; 2,2,5-trimethyl-5-pentylcyclopentanone; 2-heptylcyclopentanone; 2-pentylcyclopentanone; 2-hydroxy-3-methyl-2-cyclopenten-1-one; 3-methyl-cis-2-penten-1-yl-2-cyclopenten-1-one; 3-methyl-2-pentyl-2-cyclopenten-1-one; 3-methyl-4-cyclopentadecenone; 3-methyl-5-cyclopentadecenone; 3-methylcyclopentadecanone; 4-(1-ethoxyvinyl)-3,3,5,5-tetramethylcyclohexanone; 4-tert.-pentylcyclohexanone; 5-cyclohexadecen-1-one; 6,7-dihydro-1,1,2,3,3-pentamethyl-4(5H)-indanone; 7-cyclohexadecen-1-one; 8-cyclohexadecen-1-one; 9-cycloheptadecen-1-one; cyclopentadecanone; cyclohexadecanone;

the cycloaliphatic aldehydes, such as, for example, 2,4-dimethyl-3-cyclohexenecarbaldehyde; 2-methyl-4-(2,2,6-trimethyl-cyclohexen-1-yl)-2-butenal; 4-(4-hydroxy-4-methylpentyl)-3-cyclohexenecarbaldehyde; 4-(4-methyl-3-penten-1-yl)-3-cyclohexenecarbaldehyde;

the cycloaliphatic ketones, such as, for example, 1-(3,3-dimethylcyclohexyl)-4-penten-1-one; 1-(5,5-dimethyl-1-cyclohexen-1-yl)-4-penten-1-one; 2,3,8,8-tetramethyl-1,2,3,4,5,6,7,8-octahydro-2-naphthalenyl methyl ketone; methyl 2,6,10-trimethyl-2,5,9-cyclododecatrienyl ketone; tert.-butyl (2,4-dimethyl-3-cyclohexen-1-yl) ketone;

the esters of cyclic alcohols, such as, for example, 2-tert-butyl cyclohexylacetate; 4-tert-butyl cyclohexylacetate; 2-tert-pentyl cyclohexylacetate; 4-tert-pentyl cyclohexylacetate; decahydro-2-naphthyl acetate; 3-pentyltetrahydro-2H-pyran-4-yl acetate; decahydro-2,5,5,8a-tetramethyl-2-naphthyl acetate; 4,7-methano-3a,4,5,6,7,7a-hexahydro-5, or 6-indenyl acetate; 4,7-methano-3a,4,5,6,7,7a-hexahydro-5, or 6-indenyl propionate; 4,7-methano-3a,4,5,6,7,7a-hexahydro-5, or 6-indenyl isobutyrate; 4,7-methanooctahydro-5, or 6-indenyl acetate;

the esters of cycloaliphatic carboxylic acids, such as, for example, allyl 3-cyclohexylpropionate; allyl cyclohexyloxyacetate; cis- and trans-methyldihydrojasmonate; cis- and trans-methyljasmonate; methyl 2-hexyl-3-oxocyclopentanecarboxylate; ethyl 2-ethyl-6,6-dimethyl-2-cyclohexenecarboxylate; ethyl 2,3,6,6-tetramethyl-2-cyclohexenecarboxylate; ethyl 2-methyl-1,3-dioxolan-2-acetate;

the araliphatic alcohols, such as, for example, benzyl alcohol; 1-phenylethyl alcohol; 2-phenylethyl alcohol; 3-phenylpropanol; 2-phenylpropanol; 2-phenoxyethanol; 2,2-dimethyl-3-phenylpropanol; 2,2-dimethyl-3-(3-methylphenyl)propanol; 1,1-dimethyl-2-phenylethyl alcohol; 1,1-dimethyl-3-phenylpropanol; 1-ethyl-1-methyl-3-phenylpropanol; 2-methyl-5-phenylpentanol; 3-methyl-5-phenylpentanol; 3-phenyl-2-propen-1-ol; 4-methoxybenzyl alcohol; 1-(4-isopropylphenyl)ethanol;

the esters of araliphatic alcohols and aliphatic carboxylic acids, such as, for example, benzyl acetate; benzyl propionate; benzyl isobutyrate; benzyl isovalerate; 2-phenylethyl acetate; 2-phenylethyl propionate; 2-phenylethyl isobutyrate; 2-phenylethyl isovalerate; 1-phenylethyl acetate; alpha-trichloromethylbenzyl acetate; alpha, alpha-dimethylphenylethyl acetate; alpha, alpha-dimethylphenylethyl butyrate; cinnamyl acetate; 2-phenoxyethyl isobutyrate; 4-methoxybenzyl acetate;

the araliphatic ethers, such as, for example, 2-phenylethyl methyl ether; 2-phenylethyl isoamyl ether; 2-phenylethyl 1-ethoxyethyl ether; phenylacetaldehydedimethylacetal; phenylacetaldehydediethylacetal; hydratropaaldehydedimethylacetal; phenylacetaldehydeglycerinacetal; 2,4,6-trimethyl-4-phenyl-1,3-dioxane; 4,4a,5,9b-tetrahydroindeno[1,2-d]-m-dioxine; 4,4a,5,9b-tetrahydro-2,4-dimethylindeno[1,2-d]-m-dioxine;

the aromatic and araliphatic aldehydes, such as, for example, benzaldehyde; phenylacetaldehyde; 3-phenylpropanal; hydratropaaldehyde; 4-methylbenzaldehyde; 4-methylphenylacetaldehyde; 3-(4-ethylphenyl)-2,2-dimethylpropanal; 2-methyl-3-(4-isopropylphenyl)propanal; 2-methyl-3-(4-tert.-butylphenyl)propanal; 2-methyl-3-(4-isobutylphenyl)propanal; 3-(4-tert.-butylphenyl)propanal; cinnamaldehyde; alpha-butylcinnamaldehyde; alpha-amylcinnamaldehyde; alpha-hexylcinnamaldehyde; 3-methyl-5-phenylpentanal; 4-methoxybenzaldehyde; 4-hydroxy-3-methoxybenzaldehyde; 4-hydroxy-3-ethoxybenzaldehyde; 3,4-methylenedioxybenzaldehyde; 3,4-dimethoxybenzaldehyde; 2-methyl-3-(4-methoxyphenyl)propanal; 2-methyl-3-(4-methylenedioxyphenyl)propanal;

the aromatic and araliphatic ketones, such as, for example, acetophenone; 4-methylacetophenone; 4-methoxyacetophenone; 4-tert.-butyl-2,6-dimethylacetophenone; 4-phenyl-2-butanone; 4-(4-hydroxyphenyl)-2-butanone; 1-(2-naphthalenyl)ethanone; benzophenone; 1,1,2,3,3,6-hexamethyl-5-indanyl methyl ketone; 6-tert.-butyl-1,1-dimethyl-4-indanyl methyl ketone; 1-[2,3-dihydro-1,1,2,6-tetramethyl-3-(1-methylethyl)-1H-5-in denyl]ethanone; 5′,6′,7′,8′-tetrahydro-3′,5′,5′,6′,8′,8′-hexamethyl-2-acetonaphthone;

the aromatic and araliphatic carboxylic acids and their esters, such as, for example, benzoic acid; phenylacetic acid; methyl benzoate; ethyl benzoate; hexyl benzoate; benzyl benzoate; methylphenyl acetate; ethylphenyl acetate; geranylphenyl acetate; phenylethyl-phenyl acetate; methyl cinnamate; ethyl cinnamate; benzyl cinnamate; phenylethyl cinnamate; cinnamyl cinnamate; allylphenoxy acetate; methyl salicylate; isoamyl salicylate; hexyl salicylate; cyclohexyl salicylate; cis-3-hexenyl salicylate; benzyl salicylate; phenylethyl salicylate; methyl 2,4-dihydroxy-3,6-dimethylbenzoate; ethyl 3-phenylglycidate; ethyl 3-methyl-3-phenylglycidate;

the nitrogen-containing aromatic compounds, such as, for example, 2,4,6-trinitro-1,3-dimethyl-5-tert.-butylbenzene; 3,5-dinitro-2,6-dimethyl-4-tert.-butylacetophenone; cinnamic acid nitrile; 3-methyl-5-phenyl-2-pentenoic acid nitrile; 3-methyl-5-phenylpentanoic acid nitrile; methyl anthranilate; methyl-N-methyl anthranilate; Schiffs bases of methyl anthranilate with 7-hydroxy-3,7-dimethyloctanal, 2-methyl-3-(4-tert.-butylphenyl)propanal or 2,4-dimethyl-3-cyclohexenecarbaldehyde; 6-isopropylquinoline; 6-isobutylquinoline; 6-sec.-butylquinoline; indene; scatole; 2-methoxy-3-isopropylpyrazine; 2-isobutyl-3-methoxypyrazine;

the phenols, phenyl ethers and phenyl esters, such as, for example, estragole; anethole; eugenol; eugenyl methyl ether; isoeugenol; isoeugenyl methyl ether; thymol; carvacrol; diphenyl ether; beta-naphthyl methyl ether; beta-naphthyl ethyl ether; beta-naphthyl isobutyl ether; 1,4-dimethoxybenzene; eugenyl acetate; 2-methoxy-4-methylphenol; 2-ethoxy-5-(1-propenyl)phenol; p-cresylphenyl acetate;

the heterocyclic compounds, such as, for example, 2,5-dimethyl-4-hydroxy-2H-furan-3-one; 2-ethyl-4-hydroxy-5-methyl-2H-furan-3-one; 3-hydroxy-2-methyl-4H-pyran-4-one; 2-ethyl-3-hydroxy-4H-pyran-4-one; the lactones, such as, for example, 1,4-octanolide; 3-methyl-1,4-octanolide; 1,4-nonanolide; 1,4-decanolide; 8-decen-1,4-olide; 1,4-undecanolide; 1,4-dodecanolide; 1,5-decanolide; 1,5-dodecanolide; 1,15-pentadecanolide; cis- and trans-11-pentadecen-1,15-olide; cis- and trans-12-pentadecen-1,15-olide; 1,16-hexadecanolide; 9-hexadecen-1,16-olide; 10-oxa-1,16-hexadecanolide; 11-oxa-1,16-hexadecanolide; 12-oxa-1,16-hexadecanolide; ethylene-1,12-dodecane dioate; ethylene-1,13-tridecane dioate; coumarin; 2,3-dihydrocoumarin; octahydrocoumarin.

In a preferred configuration of a fragrance or flavoring composition according to the invention the compounds of Formula (I) according to the invention or to be used according to the invention are preferably combined with one or a plurality of, particularly preferably with two, three, four, five or more, flowery and/or fruity further fragrance and/or flavoring substances.

Accordingly, the present invention also relates to a fragrance or flavoring composition, comprising one, two, three, four, five or a plurality of fragrance and/or flavoring substances, which impart a flowery and/or fruity odor or taste.

Here through the compound of Formula (I) according to the invention or to be used according to the invention advantageously (at least to some extent) an olfactory enhancement of the flowery fragrance or flavoring substances is achieved.

Flower fragrance or flavoring compositions, with which compounds of Formula (I) according to the invention or to be used according to the invention (in particular in fragrance or flavoring compositions according to the invention) can be advantageously combined, are preferably selected from the group consisting of:

hydroxycitronellal, methoxycitronellal, cyclamen aldehyde [2-methyl-3-(4-isopropylphenyl)propanal], 1-(4-Isopropyl-cyclohexyl)ethanol (Mugetanol®), 4-tert.-butyl-α-methyl dihydrocinnamaldehyde (Lilial®), cis-hexahydrocuminyl alcohol (Mayol®), 3-[4-(1,1-dimethylethyl)phenyl]propanal (Bourgeonal®), 2,2-dimethyl-3-(3-methylphenyl)propanol (Majantol®), 3-methyl-3-(3-methylbenzyl)-butan-2-ol, 2-isobutyl-4-methyltetrahydro-2H-pyran-4-ol (Florosa®), 2-methyl-3-(3,4-methylenedioxyphenyl)propanal (Heliofolal®), 4-(4-hydroxy-4-methylpentyl)-3-cyclohexene carbaldehyde (Lyral®),4-(octahydro-4,7-methano-5H-inden-5-ylidene-butanal (Dupical®), vernaldehyde, 4-(4-methyl-3penten-1-yl)-3-cyclohexene carbaldehyde (Vertomugal®), octahydro-5-(4-methoxybutylidene)-4,7-methano-1H-indene (Mugoflor®), 2,6-dimethyl-2-heptanol(Freesiol®), 1-ethyl-1-methyl-3-phenylpropanol (Phemec®), 2,2-dimethyl-3-phenyl-1-propanol (muguet alcohol), profarnesol, dihydrofarnesol, farnesol, nerolidol, hydroxycitronellal dimethyl acetal, hexyl benzoate, geraniol, nerol, linaool, tetrahydrogeraniol, tetrahydrolinalool, ethyl linalool, geranyl tiglinate, phenethylalcohol (2-phenylethylalcohol), citronellol, rose oxide, 2-methyl-5-phenylpentanol (Rosaphen), 3-methyl-5-phenylpentanol (Phenoxanol), methyldihydrojasmonate (Hedion®, Hedione® high cis), 2-heptylcyclopentanone (Projasmon P), cis-jasmone, dihydrojasmone, cinammic alcohol (3-phenyl-2-propen-1-ol), dihydrocinammic alcohol (3-phenylpropanol), 2-methyl-4-phenyl-1,3-dioxolan (Jacinthaflor®) and dihydromyrcenol (2,6-dimethyl-7-octen-2-ol).

Furthermore, compounds of Formula (I) according to the invention or to be used according to the invention are advantageously suitable for enhancing fruity fragrance or flavoring substances, in particular fruity fragrance substances, in respect of their odor.

Fruity fragrance or flavoring substances, with which the compounds of Formula (I) according to the invention or to be used according to the invention can advantageously be combined and which accordingly are particularly preferred (further) fragrance or flavoring substances of fragrance or flavoring compositions according to the invention, are preferably selected from the group consisting of:

2-methyl-butyric acid ethyl ester, 4-(p-hydroxyphenyl)-2-butanone, ethyl-3-methyl-3-phenylglycidate, butyric acid isoamyl ester, acetic acid isoamyl ester, acetic acid-n-butyl ester, butyric acid ethyl ester, 3-methyl-butyric acid ethyl ester, n-hexanoic acid ethyl ester, n-hexanoic acid allyl ester, ethyl-2-trans-4-cis-decadienoate, 1,1-dimethoxy-2,2,5-trimethyl-4-hexane, 2,6-dimethyl-5-hepten-1-al, gamma-undecalactone, gamma-nonalactone, hexanal, 3Z-hexenal, n-decanal, n-dodecanal, citral, vanillin, ethylvanillin, maltol, ethylmaltol and mixtures of these.

Preferred fragrance or flavoring compositions comprise a total quantity of compounds of Formula (I) and/or (II) in the range 0.0001 through 70 Wt. %, preferably in the range 0.001 through 50 Wt. % and particularly preferably in the range 0.01 through 20 Wt. %, in each case in relation to the total quantity of fragrance and/or flavoring substances.

Generally further preferred are fragrance or flavoring compositions according to the invention, which comprise a total quantity of compounds of Formulas (I), (I-A) and/or (II) according to the invention or to be used according to the invention in the range 0.0001 through 15 Wt. %, preferably 0.001 through 10 Wt. % and particularly preferably 0.01 through 5 Wt. %, in relation to the total quantity of fragrance or flavoring composition.

Where the (one or a plurality of) compounds of Formula (I) according to the invention or to be used according to the invention are mainly used in order to impart to a fragrance or flavoring composition more fullness or mouthfill, freshness, (radiant) power, radiance, luster, rounding and/or naturalness and/or to enhance certain notes, in particular notes in the directions of flowery (here in particular rosy, indolic), leathery, fruity, fresh, creamy, woody, mossy and/or green, the proportion of compounds of Formula (I) is preferably quite low and in particular in the range 0.0005 through 2 Wt. %, preferably in the range 0.001 through 1 Wt. %, more preferably in the range 0.01 through 0.5 Wt. %, in relation to the total quantity of fragrance or flavoring composition. Where within the preferred concentration ranges a comparatively low concentration is selected, depending on the other components of the respective composition in many cases the inherent smells or tastes indicated above are not yet imparted.

Fragrance or flavoring compositions, containing one or a plurality of compounds of Formula (I) according to the invention or to be used according to the invention, can be used in liquid form, undiluted or thinned with a solvent for perfuming or flavoring.

Preferred solvents for this are ethanol, isopropanol, diethylene glycol monoethyl ether, glycerin, propylene glycol, 1,2-butylene glycol, dipropylene glycol, diethylphthalate, triethylcitrate, isopropyl myristate, triacetin and vegetable oils.

Furthermore, the fragrance or flavoring compositions according to the invention (as described above) can further be adsorbed by a carrier which ensures both a fine distribution of the fragrances in the product and also controlled release of these during application. Such carriers can be porous inorganic materials such as lightweight sulfate, silica gels, zeolites, gypsum, clay, granulated clay and gas concrete or organic materials such as wood and cellulose-based substances.

Fragrance or flavoring compositions according to the invention (as described above) can also be in microencapsulated, spray-dried, inclusion complex or extrusion product form and in such form can be preferably added to a product to be perfumed.

Optionally the properties of fragrance and/or flavoring compositions modified in this way can be further optimized through so-called coating with suitable materials for a controlled release of fragrance, to which end wax-like synthetic materials such as for example polyvinyl alcohol are preferably used

Microencapsulation of fragrance and/or flavoring compositions according to the invention can for example take place by the so-called coacervation method with the help of capsule materials, for example made from polyurethane-like substances or soft gelatin. The spray-dried fragrance and/or flavoring compositions can for example be produced by spray drying of an emulsion or dispersion containing the fragrance and/or flavoring compositions, wherein for the carriers modified starches, proteins, dextrin and vegetable gums can be used. Inclusion complexes for example can be produced by mixing fragrance and/or flavoring compositions according to the invention and cyclodextrins or urea derivatives in a suitable solvent, such as water. Extrusion products can result from the blending of the fragrance and/or flavoring compositions (according to the invention) with a suitable extrudable substance and by extrusion with subsequent solidification, optionally in a suitable solvent (e.g. isopropanol).

The items according to the invention containing one or a plurality of compounds of Formula (I) according to the invention or to be used according to the invention, are according to a preferred configuration produced in that the compounds of Formula (I) are incorporated as a substance, as a solution (e.g. in ethanol, water or 1,2-propylene glycol) or in the form of a mixture with a solid or liquid carrier (e.g. maltodextrin, starch, silica gel), other flavors or flavoring substances and optionally further adjuvants and/or stabilizers (e.g. natural or synthetic polysaccharides and/or vegetable gums such as modified starches or gum Arabic) in a basic preparation for nutrition, oral care or pleasure. Advantageously preparations according to the invention in the form of a solution and/or suspension or emulsion can also be transformed by spray drying into a solid preparation according to the invention (semi-finished product).

The spray-dried solid preparations according to the invention (for example items according to the invention) in the form of semi-finished products are particularly well-suited for the production of further preparations according to the invention. The spray-dried solid preparations according to the invention preferably contain 50 through 95% Wt. % of carriers, in particular of maltodextrin and/or starch, 5 through 40% of adjuvants, preferably natural or synthetic polysaccharides and/or vegetable gums such as modified starches or gum Arabic.

According to a further preferred embodiment, preparations according to the invention may be produced by incorporating one or a plurality of the compounds of Formula (I) according to the invention or to be used according to the invention and optionally other constituents of the preparation according to the invention into emulsions, into liposomes, for example starting from phosphatidyl choline, into microspheres, into nanospheres or also into capsules, granules or extrudates produced from a matrix suitable for foodstuffs and products serving for pleasure, for example produced from starch, starch derivatives (for example modified starch), cellulose or cellulose derivatives (for example hydroxypropylcellulose), other polysaccharides (for example dextrin, alginate, curdlan, carageenan, chitin, chitosan, pullulan), natural fats, natural waxes (for example beeswax, carnauba wax), or from proteins, for example gelatin or other natural products (for example shellac). In said embodiment, depending on the matrix, the products may be obtained by spray drying, spray granulation, melt granulation, coacervation, coagulation, extrusion, melt extrusion, emulsion methods, coating or other suitable encapsulation methods and optionally a suitable combination of the above-stated methods. In a further preferred method for producing a preparation according to the invention, compound(s) of Formula (I) according to the invention or to be used according to the invention is/are initially complexed with one or a plurality of suitable complexing agents, for example with cyclodextrins or cyclodextrin derivatives, preferably α- or β-cyclodextrin, and used in this complexed form.

Fragrance or flavoring compositions according to the invention are preferably used for the production of perfumed or flavored products (items).

The fragrance compositions according to the invention (as described above) can advantageously be used in concentrated form, in solutions or in modified form as described above for the production of perfumed or flavored items.

The compounds of Formula (I) according to the invention or to be used according to the invention can be incorporated into flavored products or products to be flavored, in particular preparations for nutrition, pleasure or oral care.

In a further aspect the present invention relates to a perfumed and/or flavored item comprising a fragrance and/or flavoring composition according to the invention, preferably in one of the configurations referred to as preferred or particularly preferred, or a compound of Formula (I), (I-A) or (II) according to the invention or to be used according to the invention, preferably in one of the configurations referred to as preferred or particularly preferred

Preferred items according to the invention here are perfumed items selected from the group consisting of detergents, hygiene or care products, in particular in the range of body and hair care, cosmetic and household, and flavored items, selected from the group consisting of foodstuffs, semi-luxury foods, drinks, oral care products (e.g. oral hygiene products) or pharmaceutical products.

Preferred perfumed and/or flavored items according to the invention (as described above) contain the compounds of Formula (I) according to the invention or to be used according to the invention in a total quantity of 0.0000005 Wt. % through 5 Wt. %, preferably of 0.00005 Wt. % through 2 Wt. % particularly preferably of 0.0001 Wt. % through 0.5 Wt. % in relation to the total weight of the item.

Further normal base materials, adjuvants and additives for the respective item according to the invention are preferably contained in quantities of up to 99.99 Wt. %, preferably of 10 through 95 Wt. %, in relation to the total weight of the item. The items according to the invention can further comprise water in a quantity of up to 99 Wt. %, preferably of 5 through 80 Wt. %, in relation to the total weight of the item.

Accordingly the present invention also relates to a perfumed item comprising a fragrance or flavor composition according to the invention, preferably a perfume composition according to the invention (perfume oil). Preferred items perfumed or to be perfumed are perfume extracts, eaux de parfum, eaux de toilettes, aftershaves, eaux de colognes, pre-shave products, splash colognes and perfumed freshening wipes, acidic, alkaline and neutral cleaners, such as, for example, floor cleaners, window cleaners, dishwashing detergents, bath and sanitary cleaners, scouring milk, solid and liquid WC cleaners, pulverulent and foam carpet cleaners, liquid laundry detergents, pulverulent laundry detergents, laundry pretreatment agents, such as bleaches, soaking agents and stain removers, fabric softeners, washing soaps, washing tablets, disinfectants, surface disinfectants, and air fresheners in liquid or gel form or deposited on a solid carrier, aerosol sprays, waxes and polishes, such as furniture polishes, floor waxes, shoe creams, and bodycare compositions, such as, for example, solid and liquid soaps, shower gels, shampoos, shaving soaps, shaving foams, bath oils, cosmetic emulsions of the oil-in-water, water-in-oil and water-in-oil-in-water type, such as, for example, skin creams and lotions, face creams and lotions, sunscreen creams and lotions, aftersun creams and lotions, hand creams and lotions, foot creams and lotions, depilatory creams and lotions, aftershave creams and lotions, tanning creams and lotions, haircare products, such as, for example, hairsprays, hair gels, hairsetting lotions, hair rinses, permanent and semipermanent hair colorants, hair-shaping compositions, such as cold waves and hair-smoothing compositions, hair tonics, hair creams and lotions, deodorants and antiperspirants, such as, for example, underarm sprays, roll-ons, deodorant sticks, deodorant creams, products in decorative cosmetics, such as, for example, eyeshadows, nail varnishes, foundations, lipsticks, mascara, and candles, lamp oils, joss-sticks, insecticides, repellents, propellants.

The preparations for nutrition or pleasure according to the invention are preferably for example baked goods (e.g. bread, dry biscuits, cakes, other baked goods), confectionery (e.g. chocolates, fruit gums, hard and soft caramels, chewing gum, liquorice), alcoholic or non-alcoholic drinks (e.g. coffee, tea, cocoa, drinks containing cocoa, wine, drinks containing wine, beer, drinks containing beer, liqueurs, whiskies, brandies, soft drinks containing fruit, isotonic drinks, refreshment drinks, nectars, fruit and vegetable juices, fruit or vegetable juice preparations), instant drinks, meat products (e.g. ham, fresh sausage or raw sausage preparations), eggs or egg products (dried egg, egg white, egg yolk), cereal products (e.g. breakfast cereals, muesli bars), milk products (e.g. milk drinks, milk ice, yoghurt, kefir, fresh cheese, soft cheese, hard cheese, dried milk powder, whey, butter, buttermilk), fruit preparations (e.g. jams, fruit ice, fruit sauces), vegetable preparations (e.g. ketchup, sauces, dried vegetables), snack articles (e.g. baked or fried potato crisps or potato pulp products, corn- or peanut-based extrudates), products based on fat and oil or emulsions thereof (e.g. mayonnaise, remoulade, dressings), ready meals and soups, spices, spice mixtures and also, in particular, seasonings, which are used in the snacks sector.

Preparations according to the invention can for example be in the form of semi-finished products or a spice mixture. Preparations according to the invention can also in particular serve as semi-finished products for the production of further preparations for nutrition or pleasure, in particular in spray-dried form.

The preparations according to the invention for oral care are in particular oral and/or tooth care agents such as toothpastes, tooth gels, tooth powders, mouthwashes, chewing gums and other oral care agents.

Substances and adjuvants which can also contain a perfumed and/or flavored item according to the invention, in particular a cosmetic preparation according to the invention, here preferably a topical cosmetic preparation, containing one or a plurality of compounds of Formula (I), are for example:

preservatives, preferably those described in US 2006/0089413, abrasives, antiacne and sebum reducing agents, preferably those described in WO 2008/046791, compounds against ageing of the skin, preferably those described in WO 2005/123101, antibacterial agents, anti-cellulitis agents, antidandruff agents, preferably those described in WO 2008/046795, antiphlogistic agents, irritation-preventing agents, anti-irritants (anti-inflammatory, irritation-preventing and irritation-inhibiting agents), preferably those described in WO 2007/042472 and US 2006/0089413, antimicrobial agents, preferably those described in WO 200/23101, antioxidants, preferably those described in WO 2005/123101, astringents, antiseptic agents, antistatics, binders, buffers, support materials, preferably those described in WO/2005/123101, chelating agents, preferably those described in WO 2005/123101, cell stimulants, cleansing agents, conditioning agents, depilators, surface-active substances, deodorizing agents and antiperspirants, preferably those described in WO 2005/123101, softeners, emulsifiers, preferably those described in WO 2005/12301, enzymes, ethereal oils, preferably those described in US 2008/0070825, insect repellents, preferably those described WO 2005/123101, fibers, film-formers, other fixers, foaming agents, foam stabilizers, substances to prevent foaming, foam boosters, fungicides, gelling agents, gel forming agents, preferably those described in WO 205/12301, hair care products, hair styling agents, hair straightening agents, moisture regulators (moisture-donating agents, moisturizing substance, moisture-retaining substances), preferably those described in WO 2005/123101, osmolytes, preferably those described in WO 2005/123101, compatible solutes, preferably those described in WO 01/76572 and WO 02/15868, bleaching agents, strengthening agents, stain-removing agents, optically brightening agents, impregnating agents, dirt-repellent agents, friction-reducing agents, lubricants, moisturizing crèmes, ointments, opacifying agents, plasticizing agents, covering agents, polish, gloss agents, polymers, preferably those described in WO 2008/046676, powders, proteins and protein hydrolysates, preferably those described in WO 2005/123101 and WO 2008/046676, lipid replenishing crèmes, polishing agents, skin soothing agents, skin cleansing agents, skin care agents, skin repair agents, preferably containing cholesterol and/or fatty acids and/or ceramides and/or pseudoceramides, preferably those described in WO 2006/053912, skin lightening agents, preferably those described in WO 2007/110415, skin-protecting agents, skin-softening agents, skin-cooling agents, preferably those described in WO 2005/123101, skin-warming agents, preferably those described in WO 2005/123101, stabilizers, UV-absorbing agents and UV-filters, preferably those described in WO 2005/123101, benzylidene-beta-dicarbonyl compounds, preferably those described in WO 2005/107692, alpha-benzoyl-cinnamic acid nitriles, preferably those described in WO 2006/015954, AhR-receptor antagonists, preferably those described in WO 2007/128723 and WO 2007/060256, detergents, fabric conditioners, suspending agents, skin tanning agents preferably those described in WO 2006/045760, thickening agents, vitamins, preferably those described in WO 2005/123101, oils, waxes and other fats, preferably those described in WO 2005/123101, phospholipids, preferably those described in WO 2005/123101, fatty acids (saturated fatty acids and mono- or polyunsaturated fatty acids and α-hydroxy acids and polyhydroxy-fatty acids), preferably those described in WO 2005/123101, condensers, dyestuffs and color-protecting agents and pigments, preferably those described in WO 2005/123101, anti-corrosive agents, alcohols and polyols, preferably those described in WO 2005/123101, surfactants, preferably those described in WO 2005/123101, animal extracts, yeast extracts, extracts from algae or microalgae, electrolytes, condensers, organic solvents, preferably those described in WO 2005/123101, hair growth modulating agents (hair growth promoting or hair growth inhibiting), preferably those described in EP 2168570 and EP 2193785 or silicones and silicone derivatives preferably those described in WO2008/046676.

Preferred products according to the invention are (a) perfume oils (as an example of fragrance compositions according to the invention) for surfactant-containing formulations, such as for example detergents, cleaning agents, fabric conditioners and body care products as well as (b) the corresponding surfactant-containing formulations themselves.

Particularly preferred perfumed items according to the invention are therefore washing agents or care products, in particular in the range of body and hair care, cosmetics and household products.

The preferred surfactant-containing formulations, which can be used in the context of the present invention, comprise in addition to the fragrance compositions according to the invention general substances from the class of anionic surfactants, such as for example carboxylates, sulfates, sulfonates and phosphates, the cationic surfactants, such as for example quaternary ammonium salts, amphoteric surfactants, such as for example betaines, and the non-ionic surfactants, such as for example ethoxylates and propoxylates.

Of the anionic surfactants, the sulfates and sulfonates are preferred. The preferred sulfates are those with 12 through 18 carbon atoms and a degree of ethoxylation of 1 to a maximum of 5. In particular preference is for sodium lauryl ether sulfate, preferably with an average degree of ethoxylation of 2 through 4.

Of the sulfonates in particular the linear potassium alkyl benzene sulfonates with an average of approximately 12 carbon atoms in the alkyl chain, wherein these consist of homologous radicals with 10 through 14 carbon atoms (“dodecyl benzene sulfinate”), are preferred.

Of the group of non-ionic surfactants the ethoxylated fatty alcohols, obtained by ethoxylation of alcohols with 12 through 18 carbon atoms (fatty alcohol ethoxylates with 12 through 18 C-atoms), are preferred. The degree of ethoxylation here can vary within broad limits, but particularly preferred are products with an average degree of ethoxylation of 5 through 10, in particular of 7 Mol added ethylene oxide per Mol of fatty alcohol.

Of the betaines in particular those of the acid amide type with the structure shown are preferred.

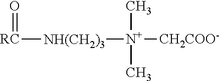

A preferred radical RC═O here is the section of the fatty acids of coconut oil in which the lauric acid at 45 through 50% is the main component.

In combination with selected surfactants the favorable properties of the compounds of Formula (I) according to the invention or to be used according to the invention are surprisingly strongly pronounced. A corresponding surfactant formulation according to the invention (as an example of an item according to the invention) comprises one or a plurality of compounds of Formula (I) and one or a plurality of surfactants selected from the group consisting of:
linear alkyl benzene sulfonates (in particular those mentioned above, such as for example the linear sodium alkyl benzene sulfonates),
fatty alcohol ethoxylates with 12 through 18 C-atoms (in particular those mentioned above, thus for example those with the degree of ethoxylation identified as preferred),
lauryl ether sulfates (in particular those mentioned above, thus for example the abovementioned sodium lauryl ether sulfate) and
betaines (in particular those mentioned above, thus for example betaines of the acid amide type with the structure shown above).

Linear alkyl benzene sulfonates and fatty alcohol ethoxylates with 12 through 18 C-atoms are preferably used alongside each other here, in particular in all-purpose detergent powders.

Similarly, lauryl ether sulfates (in particular the abovementioned sodium lauryl ether sulfate) and betaines (in particular betaines of the acid amide type with the structure shown above) are preferably used alongside each other, in particular in mild-action detergents, shampoos and shower gels.

The concentration of the surface-active substances in the surfactant formulations according to the invention is generally not critical. Preferred concentrations are dependent upon the type of surfactant and the respective application. They can for example in special bleach products be less than 1 Wt. %, but in soaps or washing powders more than 99 Wt. %.

For certain application areas in surfactant formulations according to the invention certain combinations and concentrations are preferred. Thus mixtures according to the invention (detergent formulations) are preferred in which the proportion of linear alkyl benzene sulfonates is in the range 7-10 Wt %. and/or in which the proportion of fatty alcohol ethoxylates with 12-18 C-atoms is in the range 3-6 Wt. %, in each case in relation to the total weight of the mixture. In addition mixtures according to the invention (formulations for mild-action detergents, shampoos and shower gels) are preferred, in which the proportion of sodium lauryl ether sulfate is in the range 7-13 Wt. % and/or the proportion of betaine (in particular betaine of the acid amide type with the structure shown above) is in the range 1 through 3 Wt. %, in each case in relation to the total weight of the mixture.

The items according to the invention and preparations within the meaning of the present invention may also be in the form of capsules, tablets (uncoated and coated tablets, e.g. enteric coatings), dragees, granules, pellets, solids mixtures, dispersions in liquid phases, in the form of emulsions, in the form of powders, in the form of solutions, in the form of pastes or in the form of other swallowable or chewable preparations as food supplements

Preparations according to the invention for nutrition or pleasure are preferably alcoholic or non-alcoholic drinks which also preferably comprise one or a plurality of usual common additives such as fruit components, inorganic or organic acids permitted for food purposes, antioxidants, opacifiers, loading materials, sweeteners, aromas, colorings, thickening agents, so-called “functional” additives and preservatives.

Fruit components here primarily mean fruit aromas, fruit juices, fruit purees and fruit juice concentrates. Fruit juices or fruit concentrates that can be used are those with a citrus fruit base, for example orange, lemon, grapefruit and mandarin, and other fruits, for example apple or grape. In addition fruit juices and fruit juice concentrates from soft fruit, such as blackberries, gooseberries, currants, blueberries, strawberries and raspberries can be used. Furthermore, fruit juices and fruit juice concentrates from exotic fruits, for example guava, papaya, passion fruit, mango and banana can be used.

As sweetening agents common sugars (in particular sucrose), sweeteners, sugar substitutes and sweetening compositions, as well as mixtures of these, can be used.

Suitable sweeteners are in particular acesulfame-K, aspartame, cyclamate (and Na- and Ca-salts thereof), neohesperidine dihydrochalcone, sucralose and saccharine (and Na-, K- and Ca-salts thereof). Vegetable sweeteners can also be used, for instance glycyrrhizine and thaumatine. Acesulfame-K, aspartame, cyclamate, Na-cyclamate, saccharin, Na-saccharin and sucralose are particularly preferred.

Suitable sugar substitutes are sugar alcohols, such as isomatitol (E953), lactitol (E966), maltitol, manitol (E421), sorbitol (E420), xylitol (E967) as well as mixtures thereof. Furthermore, mixtures of synthetic sweetener and dearomatized or non-dearomatized concentrated fruit preparations can be used as sweetening agents.

Suitable emulsifiers in drinks according to the invention are for example gum Arabic or modified starches, with preference here being for starch sodium octenyl succinate (E 1450).

Alcoholic or non-alcoholic drinks according to the invention can also contain terpene oils. Preferred terpene oils in the context of the present invention comprise or consist of orange, lemon and/or grapefruit oil or fractions obtainable therefrom, preferably limonene (in particular d-limonene) and/or orange oil terpenes.

Preferred loading materials used are SAIB (saccharoseacetate-isobutyrate, E 444), ester gum (E 445) and to a limited extent dammar gum and BVOs (brominated vegetable oils). These allow stable drink clouding in emulsion drinks such as for example in soft drinks or fruit juice drinks.

A drink according to the invention usually contains one or a plurality of food acids. Preferred food acids are citric acid, tartaric acid, lactic acid, phosphoric acid and malic acid. The pH of emulsion drinks according to the invention is usually in the range 3 through 5.

Alcoholic or non-alcoholic drinks according to the invention may also contain hydrocolloids as thickening agents. Suitable thickening agents are carboxymethylcellulose, Xanthan, carob gum, gellan gum, guar gum, gum Arabic, carrageenan, alginic acid, alginate, pectin and mixtures thereof.

Alcoholic or non-alcoholic drinks according to the invention can advantageously also contain so-called “functional” additives, such as for example vitamins (A, B, C, D, E, K), minerals, herb extracts, roughage, prebiotic additives, amino acids, taurine and/or caffeine.

Alcoholic or non-alcoholic drinks according to the invention can be colored with food colorings and/or coloring foods (e.g. beta-carotene).

Suitable preservatives are, for example, sorbic acid, benzoic acid and the alkaline salts thereof.

A drink according to the invention can also contain natural and synthetic antioxidants. Suitable natural antioxidants are, for example, tocopherols, L-ascorbic acid, fatty acid esters thereof, such as L-ascorbyl palmitate, gallic acid esters, and flavonides. Suitable synthetic antioxidants are, for example, tert.-butyl hydroxyanisole and tert.-butyl hydroquinone.

Preparations according to the invention can in particular be or be contained in seasonings. Suitable seasonings contain, for example, synthetic, natural or nature identical flavoring substances as well as carriers such as polysaccharides, e.g. maltodextrin, salts, e.g. cooking salt, spices, e.g. paprika and pepper, sugars or sugar substitutes or sweeteners, e.g. saccharine, and taste enhancers, e.g. monosodium glutamate and/or inosine monophosphate.

The preparations according to the invention (as described above) can be produced by incorporating the compounds of Formula (I) according to the invention or to be used according to the invention as a solution in the form of a mixture with a solid or liquid carrier in the preparation for nutrition, oral hygiene or pleasure. Advantageously the preparations according to the invention which are thus in the form of a solution can be converted by spray drying into a solid preparation.

In order to produce a preparation according to the invention (as described above) according to a preferred configuration the compounds of Formula (I) according to the invention or to be used according to the invention or preferably a flavor composition according to the invention (as described above) and optionally other components of the preparation according to the invention can also be incorporated first (that is to say prior to incorporation in the preparation) in emulsions, in liposomes, e.g. based on phosphatidylcholines, in microspheres, in nanospheres or also in capsules made from a matrix suitable for staple and semi luxury foods, e.g. starch, starch derivatives, other polysaccharides, natural fats, natural waxes or proteins, e.g. gelatin. A further preferred embodiment is characterized in that the compounds of Formula (I) according to the invention or to be used according to the invention, in particular a flavor composition according to the invention (as described above), are first complexed with a suitable complexing agent, for example with cyclodextrins or cyclodextrin derivatives, preferably beta-cyclodextrin, and used in this form.

As other components for preparations according to the invention for nutrition or pleasure there may be used further basic substances, adjuvants and additives conventional for foodstuffs or semi luxury foods, for example water, mixtures of fresh or processed, vegetable or animal base or raw substances (e.g. raw, roast, dried, fermented, smoked and/or boiled meat, egg, bone, cartilage, fish, crustaceans and shellfish, vegetables, fruits, herbs, nuts, vegetable or fruit juices or pastes or mixtures thereof), digestible or non-digestible carbohydrates (e.g. sucrose, maltose, fructose, glucose, dextrins, amylose, amylopectin, inulin, xylans, cellulose), sugar alcohols (e.g. sorbitol, mannitol, xylitol), natural or hardened fats (e.g. tallow, lard, palm oil, coconut fat, hardened vegetable fat), fatty oils (e.g. sunflower oil, groundnut oil, maize oil, thistle oil, olive oil, walnut oil, fish oil, soybean oil, sesame oil), fatty acids or their salts (e.g. potassium stearate, potassium palmitate), proteinogenic or non-proteinogenic amino acids and related compounds (e.g. taurine, creatine, creatinine), peptides, natural or processed proteins (e.g. gelatin), enzymes (e.g. peptidases, glucosidases, lipases), nucleic acids, nucleotides (inositol phosphate), taste-modulating substances (e.g. monosodium glutamate, 2-phenoxypropionic acid), emulsifiers (e.g. lecithins, diacylglycerols), stabilizers (e.g. carrageenan, alginate, locust bean flour, guar flour), preservatives (e.g. benzoic acid, sorbic acid), antioxidants (e.g. tocopherol, ascorbic acid), chelators (e.g. citric acid), organic or inorganic acidifying agents (e.g. malic acid, acetic acid, citric acid, tartaric acid, phosphoric acid), bitter substances (e.g. quinine, caffeine, limonene), sweeteners (e.g. saccharine, cyclamate, aspartame, neotame, neohesperidine dihydrochalcone), mineral salts (e.g. sodium chloride, potassium chloride, magnesium chloride, sodium phosphates), substances that prevent enzymatic browning (e.g. sulfite, ascorbic acid), essential oils, plant extracts, natural or synthetic colorings or coloring pigments (e.g. carotinoids, flavonoids, anthocyanins, chlorophyll and derivatives thereof), spices, trigeminally-active substances or plant extracts containing such trigeminally-active substances and taste modifiers.

According to a further aspect of the present invention the preparations according to the invention (as described above) are preferably used as semi-finished products for flavoring (further) preparations (for example for the production of finished products).

Tooth care products (as a basis for oral care preparations according to the invention as described above), generally comprise an abrasive system (grinding or polishing agent), such as e.g. silicas, calcium carbonates, calcium phosphates, aluminum oxides and/or hydroxyl apatites, surface-active substances, such as e.g. sodium lauryl sulfate, sodium lauryl sarcosmate and/or cocamidopropyl betaine, humectants, such as e.g. glycerol and/or sorbitol, thickeners, such as e.g. carboxymethyl cellulose, polyethylene glycols, carrageenans and/or Laponite®, sweeteners, such as for example saccharine, flavor correctors for unpleasant flavor impressions, flavor correctors for other, generally not unpleasant flavor impressions, flavor-modulating substances (e.g. inositol phosphate, nucleotides such as guanosine monophosphate, adenosine monophosphate or other substances such as sodium glutamate or 2-phenoxypropionic acid), cooling agents, such as for example menthol or menthol derivatives (e.g. L-menthol, L-menthyl lactate, L-menthly alkyl carbonate, menthone ketals, menthane carboxylic acid amides), 2,2,2-trialkyl acetic acid amides (e.g. 2.2.-diisopropyl propionic acid methyl amides), icilin and icilin derivatives, stabilizers and active ingredients, such as for example sodium fluoride, sodium monofluorophosphate, tin difluoride, quaternary ammonium fluorides, zinc citrate, zinc sulfate, tin pyrophosphate, tin dichloride, mixtures of various pyrophosphates, triclosan, cetyl pyridinium chloride, aluminum lactate, potassium citrate, potassium nitrate, potassium chloride, strontium chloride, hydrogen peroxide, aromas and/or sodium bicarbonate or odor correctors.

Chewing gums, as a preferred configuration of a preparation according to the invention for oral care (as described above), generally comprise a chewing gum base, in other words a chewing compound that becomes plastic when chewed, sugars of various types, sugar substitutes, other sweeteners, sugar alcohols, flavor correctors for unpleasant flavor impressions, other flavor modulators for other, generally not unpleasant flavor impressions, flavor-modulating substances (e.g. inositol phosphate, nucleotides such as guanosine monophosphate, adenosine monophosphate or other substances such as monosodium glutamate or 2-phenoxypropionic acid), humectants, thickeners, emulsifiers, aromas and stabilizers or odor correctors.

For the compounds of Formula (I) or mixtures or fragrance or flavoring compositions to be used by preference that stated above applies by analogy in each case.

The following examples explain the invention. Unless otherwise stated all details, in particular amounts, contents and percentages, relate to the weight.

Abbreviations used: DPG=dipropylene glycol, TEC=triethylcitrate, IPM=isopropyl myristate; BB=benzoylbenzoate; Crist.=crystalline; Abs.=absolute.

### EXAMPLE 1

### Synthesis of 2-isobutyl-4-vinyl-[1,3]dioxolane

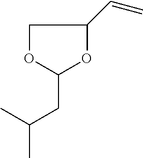

A mixture of 200 g 2-butene-1,4-diol (2.27 mol, 1.00 equiv.), 196 g isovaleraldehyde (2.27 mol, 1.00 equiv.) and 9.00 g copper(I)chloride (90.8 mmol, 0.04 equiv.) was heated in a mixture of 100 g 55% sulfuric acid and 50 g cyclohexane at 70° C. for 2 hours. Following cooling the phases were separated, the organic phase was washed with water and the aqueous phase again separated off. Following removal of the solvent the raw product obtained was purified by distillation over sodium carbonate. The product was obtained as a clear liquid (approx. 1:1-mixture of the cis- and trans-isomers) with a yield of 40% of theoretical. The boiling point of this product was 75° C. at 25 mbar.

Taste profile of this mixture of cis- and trans-2-isobutyl-4-vinyl-[1,3]dioxolane: olives, fatty, olive oil, green, spicy, tenacious, mouthfill.

The two isomers cis- or trans-2-isobutyl-4-vinyl-[1,3]dioxolane then underwent separate olfactory assessment.

Cis-isomer: MS: m/z (%)=155 (3), 126 (3), 11 (1), 99 (100), 85 (15), 71 (40), 69 (6), 57 (10), 43 (46), 41 (22), 27 (12).—¹H NMR (400 MHz, CDC₃): δ (ppm)=0.96 (d, J=6.7 Hz, 6H), 1.58 (dd, J=7.2, 5.3 Hz, 1H), 1.59 (dd, J=6.7, 5.1 Hz, 1H), 1.84 (sept, J=6.8 Hz, 1H), 3.61 (dd, J=7.9, 6.3 Hz, 1H), 3.97 (dd, J=7.9, 7.1 Hz, 1H), 4.41-4.45 (m, 1H), 4.98 (t, J=5.2 Hz, 1H), 5.20 (ddd, J=10.3, 1.5, 1.0 Hz, 1H), 5.34 (ddd, J=17.1, 1.4, 1.0 Hz, 1H), 5.84 (ddd, J=17.1, 10.3, 7.2 Hz, 1H).—¹³C NMR (100 MHz, CDCl₃): δ (ppm)=22.9 (2×CH₃), 24.6 (CH), 42.9 (CH₂), 69.5 (CH₂), 77.6 (CH), 104.4 (CH), 118.0 (CH₂), 136.1 (CH).

Olfactory description: olives, in particular green olives, natural, rosy aspects.

Trans-isomer: MS: m/z (%)=155 (2), 126 (1), 99 (100), 85 (13), 71 (33), 69 (4), 57 (7), 43 (37), 41 (18), 27 (9).—¹H NMR (400 MHz, CDCl₃): δ (ppm)=0.95 (d, J=6.7 Hz, 3H), 0.96 (d, J=6.7 Hz, 3H), 1.55 (dd, J=7.1, 5.2 Hz, 1H), 1.56 (dd, J=6.8, 5.2 Hz, 1H), 1.84 (sept, J=6.8 Hz, 1H), 3.51 (dd, J=8.3, 7.4 Hz, 1H), 4.17 (ddd, J=8.3, 6.4, 0.4 Hz, 1H), 4.44-4.50 (m, 1H), 5.05 (t, J=5.2 Hz, 1H), 5.21 (ddd, J=10.3, 1.5, 1.0 Hz, 1H), 5.33 (ddd, J=17.1, 1.5, 1.1 Hz, 1H), 5.83 (ddd, J=17.1, 10.3, 6.8 Hz, 1H).—¹³C NMR (100 MHz, CDCl₃): δ (ppm)=22.9 (2×CH₃), 24.5 (CH), 42.9 (CH₂), 70.1 (CH₂), 76.7 (CH), 103.8 (CH), 117.6 (CH₂), 135.8 (CH).

Olfactory description: olives, in particular green olives, garlic, fatty.

### EXAMPLE 2

### Synthesis of 2-secbutyl-4-vinyl-[1,3]dioxolane

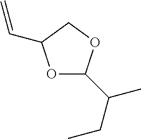

A mixture of 20.0 g 1-butene-3,4-diol (227 mmol, 1.00 equiv.), 19.5 g 2-methylbutyraldehyde (227 mmol, 1.00 equiv.) and 0.5 g p-toluolenesulfonic acid (0.27 mmol, 0.01 equiv.) was heated in 100 ml cyclohexane for 1 hour on the water separator. After cooling 50 ml water were added to the initial solution followed by agitation for 15 minutes. Then the phases were separated and the organic phase distilled over sodium carbonate.

30.6 g 2-secbutyl-4-vinyl-[1,3]dioxolane with a purity of 99.9% were isolated (4 isomers in the ratio 1:1:1:1). This corresponds to a yield of 86% of theoretical.

Boiling point: 67° C./17.5 mbar.—MS: m/z (%)=156 (M⁺, 1), 155 (1), 126 (2), 111 (1), 99 (100), 85 (4), 71 (72), 55 (27), 43 (77), 41 (54), 29 (35).—¹H NMR (400 MHz, CDCl₃): δ (ppm)=0.90-0.96 (m, 24H), 1.14-1.26 (m, 8H), 1.54-1.68 (m, 4H), 3.52 (dd, J=8.3, 7.9 Hz, 2H), 3.57 (dd, J=7.7, 6.7 Hz, 1H), 3.57 (dd, J=7.7, 6.6 Hz, 1H), 3.99 (dd, J=7.7, 6.9 Hz, 2H), 4.15 (dd, J=8.2, 6.1 Hz, 1H), 4.16 (dd, J=8.2, 6.1 Hz, 1H), 4.40-4.48 (m, 4H), 4.78 (d, J=4.3 Hz, 1H), 4.79 (d, J=4.3 Hz, 1H), 4.85 (d, J=4.3 Hz, 1H), 4.86 (d, J=4.3 Hz, 1H), 5.21 (ddd, J=10.3, 1.5, 0.9 Hz, 4H), 5.31-5.38 (m, 4H), 5.78-5.91 (m, 4H).—¹³C NMR (100 MHz, CDCl₃): δ (ppm)=11.5 (3×CH₃), 11.6 (CH₃), 13.1 (CH₃), 13.2 (CH₃), 13.3 (2×CH₃), 24.2 (CH₂), 24.3 (CH₂), 24.4 (CH₂), 26.8 (CH₂), 38.5 (CH), 38.6 (CH), 38.8 (2×CH), 69.7 (2×CH₂), 70.3 (2×CH₂), 77.1 (CH), 77.2 (CH), 77.6 (CH), 77.7 (CH), 107.8 (CH), 108.2 (3×CH), 117.8 (2×CH₂), 118.0 (2×CH₂), 135.8 (4×CH).

### EXAMPLE 3

### Synthesis of 2-isopropyl-4-vinyl-[1,3]dioxolane

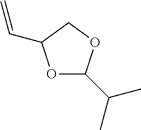

2-isopropyl-4-vinyl-[1,3]dioxolane was synthesized by analogy to Example 2, starting with 20.0 g 1-butene-3,4-diol and 16.4 g isobutyraldehyde. 24.8 g 2-isopropyl-4-vinyl-[1,3]dioxolane with a purity of 99.9% were isolated (2 isomers in the ratio 1:1). This corresponds to a yield of 77% of theoretical.

Boiling point: 56° C./27 mbar.—MS: m/z (%)=142 (M⁺, 1), 112 (2), 99 (100), 71 (74), 56 (36), 54 (11), 43 (98), 41 (48), 39 (26), 29 (14), 27 (28). ¹H NMR (400 MHz, CDC₃): δ (ppm)=0.95 (d, J=6.9 Hz, 3H), 0.96 (d, J=6.9 Hz, 3H), 0.97 (d, J=6.9 Hz, 3H), 0.98 (d, J=6.9 Hz, 3H), 1.83 (dd, J=6.9, 4.6 Hz, 1H), 1.86 (dd, J=6.9, 4.6 Hz, 1H), 3.53 (dd, J=8.1, 7.6 Hz, 1H), 3.58 (dd, J=7.8, 6.7 Hz, 1H), 3.99 (dd, J=7.7, 6.6 Hz, 1H), 4.16 (dd, J=8.2, 6.2 Hz, 1H), 4.42-4.49 (m, 2H), 4.72 (d, J=4.6 Hz, 1H), 4.79 (d, J=4.6 Hz, 1H), 5.20-5.25 (m, 2H), 5.31-5.39 (m, 2H), 5.79-5.91 (m, 2H).—¹³C NMR (100 MHz, CDCl₃): δ (ppm)=16.7 (CH₃), 16.8 (2×CH₃), 16.9 (CH₃), 32.0 (CH), 32.2 (CH), 69.8 (CH₂), 70.3 (CH₂), 77.2 (CH₂), 77.8 (CH₂), 108.5 (CH), 108.9 (CH), 117.8 (CH₂), 118.0 (CH₂), 135.7 (CH), 135.8 (CH).

### EXAMPLE 4

### Synthesis of 2-(2,4-dimethylcyclohex-3-enyl)-4-vinyl-[1,3]dioxolane

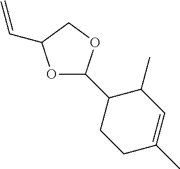

2-(2,4-dimethylcyclohex-3-enyl)-4-vinyl-[1,3]dioxolane was synthesized by analogy to Example 2, starting with 20.0 g 1-butene-3,4-diol and 31.3 g 2,4-dimethylcyclohex-3-enecarbaldehyde. 41.2 g 2-(2,4-dimethylcyclohex-3-enyl)-4-vinyl-[1,3]dioxolane with a purity of 98.5% were isolated (8 isomers). This corresponds to a yield of 86% of theoretical.

Boiling point: 77° C./0.64 mbar.—MS: m/z (%)=208 (M⁺, 1), 193 (2), 154 (3), 137 (8), 120 (21), 109 (35), 99 (64), 93 (21), 79 (19), 71 (79), 67 (41), 55 (24), 43 (100).

### EXAMPLE 5

### Synthesis of 2-(2,6-dimethylhept-5-enyl)-4-vinyl-[1,3]dioxolane

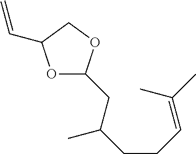

2-(2,6-dimethylhept-5-enyl)-4-vinyl-[1,3]dioxolane was synthesized by analogy to Example 2, starting with 20.0 g 1-butene-3,4-diol and 35.0 g citronellal. 7.3 g 2-(2,6-dimethylhept-5-enyl)-4-vinyl-[1,3]dioxolane with a purity of 94% were isolated (4 isomers in the ratio 1:1:1:1). This corresponds to a yield of 13% of theoretical.

Boiling point: 68° C./0.24 mbar.—MS: m/z (%)=224 (M⁺, 1), 209 (1), 193 (3), 181 (1), 167 (4), 153 (6), 139 (38), 121 (31), 109 (31), 99 (58), 81 (55), 69 (98), 55 (37), 41 (100).—¹H NMR (400 MHz, CDC₃): δ (ppm)=0.96 (d, J=6.7 Hz, 12H), 0.90-1.55 (m, 20H), 1.60 (s, 12H), 1.68 (s, 12H), 1.92-2.05 (m, 8H), 3.51 (dd, J=8.3, 7.5 Hz, 1H), 3.52 (dd, J=8.3, 7.5 Hz, 1H), 3.61 (dd, J=7.8, 7.1 Hz, 1H), 3.62 (dd, J=7.8, 7.1 Hz, 1H), 3.97 (dd, J=7.9, 7.2 Hz, 1H), 3.98 (dd, J=7.9, 7.2 Hz, 1H), 4.17 (dd, J=8.2, 6.4 Hz, 1H), 4.18 (dd, J=8.2, 6.4 Hz, 1H), 4.41-4.50 (m, 4H), 4.98-5.13 (m, 8H), 5.19-5.24 (m, 4H), 5.31-5.37 (m, 4H), 5.79-5.90 (m, 4H).—¹³C NMR (100 MHz, CDCl₃): δ (ppm)=17.6 (2×CH₃), 18.6 (CH₃), 18.7 (CH₃), 19.6 (CH₃), 19.8 (2×CH₃), 19.9 (CH₃), 25.4 (4×CH₂), 25.7 (4×CH₃), 29.0 (3×CH), 29.2 (CH), 36.9 (CH₂), 37.4 (CH₂), 37.5 (2×CH₂), 41.2 (4×CH₂), 69.5 (CH₂), 69.6 (CH₂), 70.1 (CH₂), 70.2 (CH₂), 77.6 (4×CH), 103.8 (CH), 103.9 (CH), 104.4 (2×CH), 117.6 (2×CH₂), 117.9 (2×CH₂), 124.2 (CH), 124.7 (3×CH), 131.2 (4×C_{quart.}), 135.8 (2×CH), 136.1 (2×CH).

### EXAMPLE 6

### Synthesis of 2-ethyl-2-(2-methylbutyl)-4-vinyl-[1,3]dioxolane

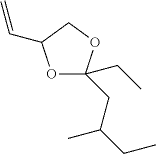

2-ethyl-2-(2-methylbutyl)-4-vinyl-[1,3]dioxolane was synthesized by analogy to Example 2, starting with 20.0 g 1-butene-3,4-diol and 29.1 g 5-methyl-3-heptanone. 28.9 g 2-Ethyl-2-(2-methylbutyl)-4-vinyl-[1,3]dioxolane with a purity of 97% were isolated (4 isomers in the ratio 1:1:1:1). This corresponds to a yield of 62% of theoretical.

Boiling point: 109° C./14 mbar.—MS: m/z (%)=169 (13), 127 (52), 113 (10), 99 (31), 86 (4), 71 (29), 57 (100), 43 (24), 29 (31).—¹H NMR (400 MHz, CDCl₃): δ (ppm)=0.86 (2 t, J=7.5 Hz, 6H), 0.87 (t, J=7.5 Hz, 3H), 0.87 (t, J=7.5 Hz, 3H), 0.92 (3 t, J=7.5 Hz, 9H), 0.93 (t, J=7.5 Hz, 3H), 0.94 (d, J=6.4 Hz, 6H), 0.95 (t, J=6.4 Hz, 6H), 1.13-1.24 (m, 4H), 1.35-1.48 (m, 8 H), 1.50-1.58 (m, 4H), 1.63-1.72 (m, 12H), 3.53 (t, J=8.0 Hz, 1H), 3.55 (2 t, J=8.0 Hz, 2H), 3.56 (t, J=8.1 Hz, 1H), 4.08 (dd, J=8.0, 6.3 Hz, 1H), 4.09 (dd, J=8.0, 6.3 Hz, 1H), 4.10 (2 dd, J=8.0, 6.3 Hz, 2H), 4.43-4.52 (m, 4H), 5.20 (3 ddd, J=10.3, 1.5, 1.0 Hz, 3H), 5.21 (ddd, J=10.3, 1.5, 1.0 Hz, 1H), 5.34 (3 ddd, J=17.1, 1.4, 1.0 Hz, 3H), 5.35 (ddd, J=17.1, 1.4, 1.0 Hz, 1H), 5.82 (ddd, J=17.2, 10.3, 7.1 Hz, 2H), 5.83 (ddd, J=17.2, 10.3, 7.1 Hz, 2H).—¹³C NMR (100 MHz, CDCl₃): δ (ppm)=8.1 (2×CH₃), 8.2 (2×CH₃), 11.3 (2×CH₃), 11.4 (2×CH₃), 20.5 (3×CH₃), 20.7 (CH₃), 30.2 (CH), 30.3 (2×CH₂), 30.3 (CH), 30.4 (CH), 30.4 (2×CH₂), 30.6 (CH), 30.7 (2×CH₂), 30.8 (2×CH₂), 43.1 (2×CH₂), 43.4 (CH₂), 43.5 (CH₂), 69.4 (CH₂), 69.5 (2×CH₂), 69.6 (CH₂), 77.4 (CH), 77.5 (CH), 77.6 (CH), 77.7 (CH), 113.4 (2×CH), 113.5 (2×CH), 117.9 (2×CH₂), 118.0 (CH₂), 118.1 (CH₂), 135.9 (3×CH), 136.0 (CH).

### EXAMPLE 7

### Synthesis of 2-(2,4,4-trimethylpentyl)-4-vinyl-[1,3]dioxolane

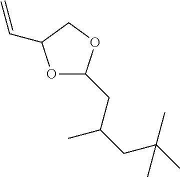

2-(2,4,4-trimethylpentyl)-4-vinyl-[1,3]dioxolane was synthesized by analogy to Example 2, starting with 20.0 g 1-butene-3,4-diol and 32.3 g 3,5,5-trimethylhexanal. 41.2 g 2-(2,4,4-trimethylpentyl)-4-vinyl-[1,3]dioxolane with a purity of 97% were isolated (4 isomers in the ratio 1:1:1:1). This corresponds to a yield of 83% of theoretical.

Boiling point: 62° C./0.88 mbar.—MS: m/z (%)=138 (1), 109 (12), 99 (100), 83 (19), 71 (40), 57 (33), 43 (38), 29 (12).—¹H NMR (400 MHz, CDCl₃): δ (ppm)=0.90 (3 s, 36H), 0.99 (4 d, J=6.6 Hz, 12H), 1.08 (dd, J=14.0, 6.1 Hz, 2H), 1.09 (dd, J=14.0, 6.1 Hz, 2H), 1.27 (dd, J=14.0, 3.9 Hz, 2H), 1.28 (dd, J=14.0, 3.9 Hz, 2H), 1.45-1.58 (m, 4H), 1.62-1.71 (m, 4H), 1.71-1.81 (m, 4H), 3.51 (dd, J=8.3, 7.4 Hz, 2H), 3.61 (dd, J=7.9, 6.4 Hz, 1H), 3.61 (dd, J=7.9, 6.3 Hz, 1H), 3.96 (dd, J=7.8, 7.0 Hz, 1H), 3.97 (dd, J=7.9, 7.0 Hz, 1H), 4.17 (dd, J=8.3, 6.4 Hz, 1H), 4.17 (ddd, J=8.2, 6.5, 1.4 Hz, 1H), 4.40-4.45 (m, 2H), 4.44-4.51 (m, 2H), 4.96 (dd, J=5.7, 4.5 Hz, 1H), 4.97 (t, J=5.0 Hz, 1H), 5.03 (dd, J=5.6, 4.7 Hz, 1H), 5.04 (t, J=5.1 Hz, 1 H), 5.20 (2 ddd, J=10.3, 1.5, 1.1 Hz, 2H), 5.21 (ddd, J=10.3, 1.5, 1.1 Hz, 1H), 5.22 (ddd, J=10.3, 1.5, 1.1 Hz, 1H), 5.33 (2 ddd, J=17.2, 1.5, 1.1 Hz, 2H), 5.34 (2 ddd, J=17.2, 1.5, 1.1 Hz, 2H), 5.82 (2 ddd, J=17.2, 10.3, 7.2 Hz, 2H), 5.84 (ddd, J=17.2, 10.3, 7.2 Hz, 1H), 5.85 (ddd, J=17.2, 10.3, 7.2 Hz, 1H).—¹³C NMR (100 MHz, CDCl₃): δ (ppm)=22.9 (CH₃), 23.0 (3×CH₃), 25.8 (4×CH), 30.0 (12×CH₃), 31.2 (4×C_{quart.}), 43.5 (3×CH₂), 43.6 (CH₂), 51.4 (CH₂), 51.5 (3×CH₂), 69.5 (CH₂), 69.6 (CH₂), 70.1 (2×CH₂), 76.6 (CH), 76.7 (CH), 77.5 (CH), 77.6 (CH), 103.8 (CH), 103.9 (CH), 104.4 (2×CH), 117.4 (CH₂), 117.6 (CH₂), 117.8 (CH₂), 117.9 (CH₂), 135.8 (2×CH), 136.1 (2×CH).

### EXAMPLE 8

### Synthesis of 2-(3,5-dimethylhex-4-enyl)-2-methyl-4-vinyl-[1,3]dioxolane

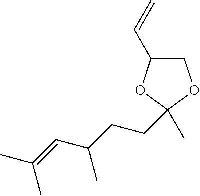

2-(3,5-dimethylhex-4-enyl)-2-methyl-4-vinyl-[1,3]dioxolane was synthesized by analogy to Example 2, starting with 20.0 g 1-butene-3,4-diol and 35.0 g 5,7-dimethyloct-6-en-2-one. 35.5 g 2-(3,5-dimethylhex-4-enyl)-2-methyl-4-vinyl-[1,3]dioxolane with a purity of 99.4% were isolated (4 isomers in the ratio 1:1:1:1). This corresponds to a yield of 69% of theoretical.

Boiling point: 104° C./7.7 mbar.—MS: m/z (%)=209 (2), 153 (6), 123 (9), 113 (38), 96 (40), 83 (13), 71 (15), 55 (25), 43 (100), 29 (6).—¹H NMR (400 MHz, CDCl₃): δ (ppm)=0.91 (d, J=6.7 Hz, 6H), 0.92 (d, J=6.7 Hz, 6H), 1.20-1.65 (m, 16H), 1.32 (s, 6H), 1.35 (s, 6H), 1.59 (2 s, 12 H), 1.67 (2 s, 12H), 2.23-2.35 (m, 4H), 3.53 (t, J=7.9 Hz, 1H), 3.54 (t, J=8.0 Hz, 1H), 3.59 (t, J=8.2 Hz, 2H), 4.08 (dd, J=8.0, 6.2 Hz, 1H), 4.09 (dd, J=8.0, 6.2 Hz, 1H), 4.09 (dd, J=8.2, 6.2 Hz, 1H), 4.10 (dd, J=8.2, 6.2 Hz, 1H), 4.41-4.48 (m, 2H), 4.48-4.54 (m, 2H), 4.85-4.89 (m, 4H), 5.19-5.23 (m, 4H), 5.31-5.38 (m, 4H), 5.82 (ddd, J=17.1, 10.3, 7.2 Hz, 2H), 5.82 (ddd, J=17.3, 10.1, 7.2 Hz, 1H), 5.83 (ddd, J=17.3, 10.1, 7.2 Hz, 1H).—¹³C NMR (100 MHz, CDCl₃): δ (ppm)=18.0 (4×CH₃), 21.4 (4×CH₃), 25.8 (4×CH₃), 31.9 (2×CH₂), 32.0 (2×CH₂), 32.6 (4×CH), 37.4 (2×CH₂), 37.8 (2×CH₂), 69.3 (2×CH₂), 69.5 (CH₂), 69.6 (CH₂), 77.1 (CH), 77.2 (CH), 77.8 (2×CH), 111.2 (2×C_{quart.}), 111.3 (2×C_{quart.}), 118.0 (2×CH₂), 118.1 (2×CH₂), 130.1 (4×C_{quart.}), 131.1 (4×C_{quart.}), 135.7 (2×CH), 136.1 (2×CH).

### EXAMPLE 9

### Synthesis of 2-methyl-2-(4-methylpent-3-enyl)-4-vinyl-[1,3]dioxolane

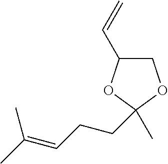

2-methyl-2-(4-methylpent-3-enyl)-4-vinyl-[1,3]dioxolane was synthesized by analogy to Example 2, starting with 20.0 g 1-butene-3,4-diol and 28.6 g 6-methylhept-5-en-2-one. 36.5 g 2-methyl-2-(4-methylpent-3-enyl)-4-vinyl-[1,3]dioxolane with a purity of 96% were isolated (2 isomers in the ratio 1:1). This corresponds to a yield of 78% of theoretical.

Boiling point: 80° C./1.4 mbar.—MS: m/z (%)=196 (M⁺, 2), 125 (4), 113 (30), 108 (4), 93 (3), 83 (5), 69 (14), 55 (8), 43 (100), 27 (5).—¹H NMR (400 MHz, CDC₃): δ (ppm)=1.35 (s, 3H), 1.38 (s, 3H), 1.60-1.63 (m, 6H), 1.67-1.69 (m, 6H), 2.14-2.3 (m, 8H), 3.55 (t, J=8.0 Hz, 1H), 3.61 (t, J=8.1 Hz, 1H), 4.11 (dd, J=8.0, 6.2 Hz, 1H), 4.12 (dd, J=8.1, 6.2 Hz, 1H), 4.46 (ddd, J=8.1, 6.2, 1.0 Hz, 1H), 4.53 (ddd, J=8.1, 6.2, 1.0 Hz, 1H), 5.07-5.15 (m, 2H), 5.21 (ddd, J=10.3, 1.5, 0.8 Hz, 1H), 5.22 (ddd, J=10.3, 1.5, 0.9 Hz, 1H), 5.35 (ddd, J=17.1, 1.4, 1.0 Hz, 1H), 5.36 (ddd, J=17.1, 1.4, 1.0 Hz, 1H), 5.83 (ddd, J=17.1, 10.2, 7.1 Hz, 1H), 5.83 (ddd, J=17.1, 10.3, 7.1 Hz, 1H).—¹³C NMR (100 MHz, CDCl₃): δ (ppm)=17.6 (2×CH₃), 22.7 (CH₂), 22.8 (CH₂), 24.2 (CH₃), 24.8 (CH₃), 25.7 (2×CH₃), 39.3 (CH₂), 39.8 (CH₂), 69.3 (CH₂), 69.6 (CH₂), 77.2 (CH), 77.9 (CH), 110.8 (C_{quart.}), 110.9 (C_{quart.}), 118.1 (2×CH₂), 124.0 (CH), 124.1 (CH), 131.6 (C_{quart.}), 131.7 (C_{quart.}), 135.6 (CH), 136.1 (CH).

### EXAMPLE 10

### Synthesis of 8-tertbutyl-2-vinyl-1,4-dioxaspiro-[4.5]-decane

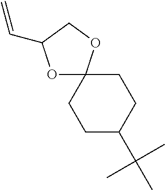

8-tertbutyl-2-vinyl-1,4-dioxaspiro-[4.5]-decane was synthesized by analogy to Example 2, starting with 20.0 g 1-butene-3,4-diol and 35.0 g 4-tertbutylcyclohexanone. 43.7 g 8-tertbutyl-2-vinyl-1,4-dioxaspiro-[4.5]-decane with a purity of 99% were isolated (2 isomers in the ratio 1:1). This corresponds to a yield of 79% of theoretical.

Boiling point: 82° C./0.95 mbar.—MS: m/z (%)=194 (1), 167 (2), 153 (18), 139 (5), 125 (100), 112 (3), 97 (5), 84 (6), 69 (12), 55 (94), 41 (28), 29 (6).—H NMR (400 MHz, CDCl₃): δ (ppm)=0.86 (s, 18H), 0.98-1.10 (m, 2H), 1.21-1.63 (m, 16H), 3.59 (dd, J=8.2, 7.4 Hz, 1H), 3.60 (t, J=7.7 Hz, 1H), 4.09 (dd, J=8.1, 6.3 Hz, 2H), 5.46-5.54 (m, 2H), 5.18-5.22 (m, 2H), 5.34 (ddd, J=17.2, 1.5, 1.0 Hz, 1H), 5.35 (ddd, J=17.2, 1.5, 1.0 Hz, 1H), 5.82 (ddd, J=17.2, 10.1, 7.0 Hz, 1H), 5.84 (ddd, J=17.2, 10.3, 7.0 Hz, 1H).—¹³C NMR (100 MHz, CDCl₃): δ (ppm)=24.7 (2×CH₂), 24.8 (CH₂), 24.9 (CH₂), 27.7 (6×CH₃), 32.3 (2×C_{quart.}), 35.3 (CH₂), 35.7 (CH₂), 36.1 (CH₂), 36.6 (CH₂), 47.1 (CH), 47.4 (CH), 68.9 (CH₂), 69.0 (CH₂), 76.7 (CH), 77.2 (CH), 110.0 (2×C_{quart.}), 117.7 (CH₂), 117.9 (CH₂), 136.3 (CH), 136.4 (CH).

### EXAMPLE 11

### Synthesis of 2-(2,6-dimethylhepta-1,5-dienyl-4-vinyl-[1,3]dioxolane

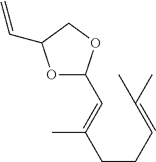

2-(2,6-dimethylhepta-1,5-dienyl-4-vinyl-[1,3]dioxolane was synthesized by analogy to Example 2, starting with 20.0 g 1-butene-3,4-diol and 34.5 g Citral. 17.9 g 2-(2,6-dimethylhepta-1,5-dienyl-4-vinyl-[1,3]dioxolane with a purity of 93% were isolated (4 isomers in the ratio 1.5:1.5:1:1). This corresponds to a yield of 33% of theoretical.

Boiling point: 107° C./2.1 mbar.—MS: m/z (%)=207 (1), 179 (1), 165 (6), 152 (11), 135 (7), 121 (11), 109 (18), 99 (21), 81 (15), 69 (87), 55 (26), 41 (100).—¹H NMR (400 MHz, CDC₃): δ (ppm)=1.60-1.61 (m, 15H), 1.68-1.69 (m, 15H), 1.77-1.80 (m, 15H), 2.02-2.20 (m, 20H), 3.51 (dd, J=7.9, 7.2 Hz, 1H), 3.55 (dd, J=8.3, 7.4 Hz, 1.5H), 3.65 (dd, J=7.8, 6.3 Hz, 1H), 3.66 (dd, J=7.8, 6.3 Hz, 1.5H), 4.00 (dd, J=7.8, 7.0 Hz, 1H), 4.01 (dd, J=7.8, 7.0 Hz, 1.5H), 4.20 (dd, 8.2, 6.0 Hz, 1H), 4.22 (dd, J=8.3, 6.3 Hz, 1.5H), 4.43-4.56 (m, 5H), 5.07-5.15 (m, 5H), 5.20-5.40 (m, 15H), 5.57 (d, J=7.5 Hz, 1H), 6.60 (d, J=7.1 Hz, 1.5H), 5.64 (d, J=7.5 Hz, 1H), 5.67 (d, J=7.2 Hz, 1.5H), 5.80-5.92 (m, 5H).—¹³C NMR (100 MHz, CDCl₃): δ (ppm)=17.0 (CH₃), 17.7 (CH₃), 23.6 (CH₃), 25.7 (CH₃), 26.1 (CH₂), 26.9 (CH₂), 32.6 (CH₂), 39.5 (CH₂), 76.8 (CH), 76.9 (CH), 77.7 (CH), 101.1 (CH), 100.4 (CH), 100.6 (CH), 101.0 (CH), 117.5 (CH₂), 117.7 (CH₂), 118.1 (CH₂), 118.2 (CH₂), 120.9 (CH), 122.0 (CH), 123.6 (CH), 123.7 (CH), 131.9 (C_{quart.}), 132.2 (C_{quart.}), 135.6 (CH), 135.7 (CH), 144.7 (C_{quart.}), 144.8 (C_{quart.}), 144.9 (C_{quart.}), 145.1 (C_{quart.}).

### EXAMPLE 12

### Synthesis of 2-[2-(4-methylcyclohex-3-enyl)-propyl]-4-vinyl-[1,3]dioxolane

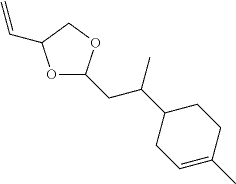

2-[2-(4-methylcyclohex-3-enyl)-propyl]-4-vinyl-[1,3]dioxolane was synthesized by analogy to Example 2, starting with 20.0 g 1-butene-3,4-diol and 37.7 g limonenal. 40.0 g 2-[2-(4-methylcyclohex-3-enyl)-propyl]-4-vinyl-[1,3]dioxolane with a purity of 98% were isolated (8 isomers). This corresponds to a yield of 73% of theoretical.

Boiling point: 90° C./0.22 mbar.—MS: m/z (%)=235 (1), 205 (6), 179 (2), 165 (4), 161 (2), 148 (65), 139 (24), 133 (56), 121 (64), 105 (42), 93 (79), 81 (44), 71 (99), 67 (47), 55 (47), 43 (100).

### EXAMPLE 13

### Synthesis of 2-phenyl-4-vinyl-[1,3]dioxolane

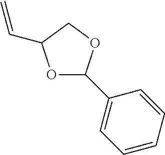

2-phenyl-4-vinyl-[1,3]dioxolane was synthesized by analogy to Example 2, starting with 25.0 g 1-butene-3,4-diol and 30.1 g benzaldehyde. 31.1 g 2-phenyl-4-vinyl-[1,3]dioxolane with a purity of 96% were isolated (2 isomers in the ratio 1:1). This corresponds to a yield of 60% of theoretical.

Boiling point: 73° C./0.70 mbar.—MS: m/z (%)=176 (M⁺, 3), 175 (16), 145 (7), 131 (3), 117 (24), 105 (78), 99 (5), 91 (28), 77 (46), 71 (10), 63 (7), 54 (100), 51 (24), 39 (19), 27 (13).—¹H NMR (400 MHz, CDCl₃): δ (ppm)=3.72 (dd, J=8.2, 7.3 Hz, 1H), 3.79 (dd, J=7.9, 6.7 Hz, 1 H), 4.17 (dd, J=7.9, 6.9 Hz, 1H), 4.31 (ddd, J=8.2, 6.3, 0.3 Hz, 1H), 4.61-4.69 (m, 2H), 5.26 (ddd, J=10.3, 1.3, 0.9 Hz, 1H), 5.27 (ddd, J=10.4, 1.3, 1.1 Hz, 1H), 5.40 (ddd, J=17.1, 1.3, 1.0 Hz, 1H), 5.41 (ddd, J=17.1, 1.3, 1.1 Hz, 1H), 5.87 (s, 1H), 5.92 (ddd, J=17.1, 10.4, 2.5 Hz, 1H), 5.94 (ddd, J=17.2, 10.3, 2.8 Hz, 1H), 5.98 (s, 1H), 7.35-7.40 (m, 6H), 7.47-7.52 (m, 4H).—¹³C NMR (100 MHz, CDCl₃): δ (ppm)=70.0 (CH₂), 70.4 (CH₂), 77.3 (CH), 78.3 (CH), 103.7 (CH), 104.4 (CH), 118.1 (CH₂), 118.4 (CH₂), 126.4 (2×CH), 126.6 (2×CH), 128.3 (4×CH), 129.1 (CH), 129.3 (CH), 135.3 (CH), 135.4 (CH), 137.6 (C_{quart.}), 138.0 (C_{quart.}).

### EXAMPLE 14

### Synthesis of 2-cyclohexyl-4-vinyl-[1,3]dioxolane

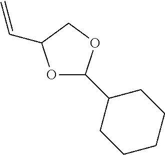

2-cyclohexyl-4-vinyl-[1,3]dioxolane was synthesized by analogy to Example 2, starting with 25.0 g 1-butene-3,4-diol and 31.8 g cyclohexane carbaldehyde. 38.3 g 2-cyclohexyl-4-vinyl-[1,3]dioxolane with a purity of 97% were isolated (2 isomers in the ratio 2:1). This corresponds to a yield of 72% of theoretical.

Boiling point: 67° C./1.6 mbar.—MS: m/z (%)=152 (1), 134 (1), 126 (1), 108 (1), 99 (100), 81 (10), 71 (39), 67 (9), 55 (23), 43 (32), 27 (10).—¹H NMR (400 MHz, CDCl₃): δ (ppm)=1.06-1.28 (m, 15H), 1.50-1.70 (m, 6H), 1.72-1.85 (m, 12H), 3.51 (dd, J=8.2, 7.5 Hz, 1H), 3.56 (dd, 7.7, 6.6 Hz, 2H), 3.98 (dd, J=7.8, 6.9 Hz, 2H), 4.14 (dd, J=8.2, 5.0 Hz, 1H), 4.39-4.46 (m, 3H), 4.69 (d, J=5.0 Hz, 2H), 4.77 (d, J=4.9 Hz, 1H), 5.21 (ddd, J=10.3, 1.5, 1.0 Hz, 3H), 5.33 (ddd, J=17.1, 1.4, 1.2 Hz, 1H), 5.34 (ddd, J=17.2, 1.4, 1.0 Hz, 2H), 5.82 (ddd, J=17.1, 10.4, 7.0 Hz, 1H), 5.83 (ddd, J=17.2, 10.2, 7.2 Hz, 2H).—¹³C NMR (100 MHz, CDCl₃): δ (ppm)=25.7 (CH₂), 25.8 (CH₂), 26.4 (CH₂), 27.2 (CH₂), 27.3 (CH₂), 27.4 (CH₂), 41.8 (CH), 42.0 (CH), 69.7 (CH₂), 70.2 (CH₂), 77.1 (CH), 77.6 (CH), 107.8 (CH), 108.2 (CH), 117.8 (CH₂), 118.0 (CH₂), 135.7 (CH), 135.8 (CH).

### EXAMPLE 15

### Synthesis of 2-(2,2,3-trimethylcyclopent-3-enylmethyl)-4-vinyl-[1,3]dioxolane

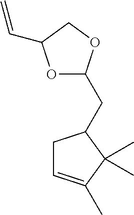

2-(2,2,3-trimethylcyclopent-3-enylmethyl)-4-vinyl-[1,3]dioxolane was synthesized by analogy to Example 2, starting with 25.0 g 1-butene-3,4-diol and 43.2 g campholene aldehyde. 43.8 g 2-(2,2,3-trimethylcyclopent-3-enylmethyl)-4-vinyl-[1,3]dioxolane with a purity of 97% were isolated (4 isomers in the ratio 2:2:1:1). This corresponds to a yield of 71% of theoretical.

Boiling point: 87° C./1.0 mbar.—MS: m/z (%)=168 (4), 153 (24), 136 (7), 119 (4), 108 (100), 93 (64), 79 (14), 67 (18), 54 (17), 43 (53).—¹H NMR (400 MHz, CDCl₃): δ (ppm)=0.76 (s, 6H), 0.77 (s, 12H), 0.98 (s, 6H), 0.99 (s, 12H), 1.60-1.61 (s, 18H), 1.65-1.85 (m, 14H), 1.88-2.02 (m, 10H), 2.31-2.42 (m, 6H), 3.52 (dd, J=8.3, 7.4 Hz, 1H), 3.53 (dd, J=8.3, 7.4 Hz, 1H), 3.62 (dd, J=7.8, 6.4 Hz, 2H), 3.63 (dd, J=7.8, 6.4 Hz, 2H), 3.98 (dd, J=7.8, 7.0 Hz, 2H), 3.99 (dd, J=7.8, 7.0 Hz, 2H), 4.18 (dd, J=8.3, 6.4 Hz, 1H), 4.19 (dd, J=8.3, 6.4 Hz, 1H), 4.42-4.53 (m, 6H), 4.98 (dd, J=4.0, 0.9 Hz, 2H), 4.99 (dd, J=4.6, 0.9 Hz, 2H), 5.05 (dd, J=4.1, 1.7 Hz, 1H), 5.06 (dd, J=4.1, 1.7 Hz, 1H), 5.19-5.25 (m, 12H), 5.33 (ddd, J=17.1, 1.3, 0.7 Hz, 1H), 5.34 (ddd, J=17.1, 1.3, 0.7 Hz, 2H), 5.34 (dd, J=17.1, 1.2 Hz, 1H), 5.34 (dd, J=17.1, 1.2 Hz, 2H), 5.83 (ddd, J=17.1, 10.3, 6.8 Hz, 1H), 5.84 (ddd, J=17.1, 10.3, 6.8 Hz, 1 H), 5.85 (ddd, J=17.2, 10.3, 7.1 Hz, 2H), 5.86 (ddd, J=17.1, 10.4, 7.2 Hz, 2H).—¹³C NMR (100 MHz, CDCl₃): δ (ppm)=12.6 (CH₃), 19.7 (CH₃), 25.5 (CH₃), 34.7 (CH₂), 35.7 (CH₂), 35.8 (CH₂), 45.8 (CH), 46.9 (C_{quart.}), 69.5 (CH₂), 69.7 (CH₂), 70.1 (CH₂), 70.2 (CH₂), 76.7 (CH), 76.9 (CH), 77.6 (CH), 77.7 (CH), 104.6 (CH), 104.7 (CH), 105.1 (CH), 105.3 (CH), 117.5 (CH₂), 117.9 (CH₂), 121.8 (CH), 135.8 (CH), 136.0 (CH), 148.2 (C_{quart.}).

### EXAMPLE 16

### Synthesis of 2-vinyl-1,4-dioxaspiro[4.5]decane

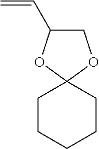

2-vinyl-1,4-dioxa-spiro[4.5]decane was synthesized by analogy to Example 2, starting with 25.0 g 1-butene-3,4-diol and 27.9 g cyclohexanone. 32.9 g 2-vinyl-1,4-dioxaspiro[4.5]decane with a purity of 98% were isolated. This corresponds to a yield of 66% of theoretical.

Boiling point: 73° C./8.9 mbar.—MS: m/z (%)=168 (M⁺, 11), 139 (13), 125 (56), 112 (14), 97 (7), 84 (8), 69 (10), 55 (100), 41 (18), 27 (9).—¹H NMR (400 MHz, CDC₃): δ (ppm)=1.35-1.45 (m, 2H), 1.55-1.67 (m, 8H), 3.60 (dd, J=8.1, 7.5 Hz, 1H), 4.01 (dd, J=8.1, 6.2 Hz, 1H), 4.50 (dddd, J=7.5, 7.2, 6.2, 0.9 Hz, 1H), 5.21 (ddd, J=10.3, 1.5, 0.9 Hz, 1H), 5.35 (ddd, J=17.1, 1.5, 1.0 Hz, 1H), 5.83 (ddd, J=17.1, 10.3, 7.2 Hz, 1H).—¹³C NMR (100 MHz, CDCl₃): δ (ppm)=23.9 (CH₂), 24.0 (CH₂), 25.2 (CH₂), 35.5 (CH₂), 36.3 (CH₂), 69.0 (CH₂), 77.0 (CH), 110.0 (C_{quart.}), 117.9 (CH₂), 136.2 (CH).

### EXAMPLE 17

### Synthesis of 7-methyl-2-vinyl-1,4-dioxaspiro[4.5]decane

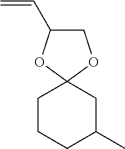

7-methyl-2-vinyl-1,4-dioxaspiro[4.5]decane was synthesized by analogy to Example 2, starting with 25.0 g 1-butene-3,4-diol and 31.8 g 3-methylcyclohexanone. 38.1 g 7-methyl-2-vinyl-1,4-dioxaspiro[4.5]decane with a purity of 96% were isolated (4 isomers in the ratio 1:1:1:1). This corresponds to a yield of 71% of theoretical.

Boiling point: 56° C./2.1 mbar.—MS: m/z (%)=182 (M⁺, 5), 167 (2), 152 (2), 139 (64), 125 (18), 11 (15), 95 (15), 84 (7), 69 (100), 55 (66), 41 (30), 27 (11).—¹H NMR (400 MHz, CDCl₃): δ (ppm)=0.90 (d, J=6.4 Hz, 6H), 0.91 (d, J=6.4 Hz, 3H), 0.92 (d, J=6.4 Hz, 3H), 1.10-1.82 (m, 36H), 3.56-3.62 (m, 4H), 4.07-4.13 (m, 4H), 4.45-4.54 (m, 4H), 5.17-5.23 (m, 4H), 5.31-5.38 (m, 4H), 5.78-5.88 (m, 4H).—¹³C NMR (100 MHz, CDCl₃): δ (ppm)=22.3 (3×CH₃), 22.4 (CH₃), 23.1 (CH₂), 23.2 (CH₂), 23.3 (CH₂), 23.4 (CH₂), 30.3 (CH), 30.4 (CH), 30.5 (CH), 30.7 (CH), 33.9 (4×CH₂), 34.6 (CH₂), 34.9 (CH₂), 35.3 (CH₂), 35.9 (CH₂), 43.6 (CH₂), 44.1 (CH₂), 44.4 (CH₂), 44.9 (CH₂), 68.8 (CH₂), 68.9 (CH₂), 69.1 (2×CH₂), 76.9 (CH), 77.0 (CH), 77.2 (2×CH), 110.4 (4×C_{quart.}), 117.8 (CH₂), 117.9 (2×CH₂), 118.0 (CH₂), 136.1 (CH), 136.2 (2×CH), 136.3 (CH).

### EXAMPLE 18

### Synthesis of 2-(2-methylpropenyl)-4-vinyl-[1,3]dioxolane

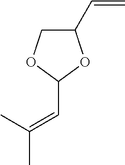

2-(2-methylpropenyl)-4-vinyl-[1.3]dioxolane was synthesized by analogy to Example 2, starting with 25.0 g 1-butene-3,4-diol and 23.9 g 3-methylbut-2-enal. 5.7 g 2-(2-methylpropenyl)-4-vinyl-[1.3]dioxolane with a purity of 96% were isolated (2 isomers in the ratio 1:1). This corresponds to a yield of 13% of theoretical.

Boiling point: 95° C./1.8 mbar.—MS: m/z (%)=153 (2), 139 (2), 124 (1), 109 (5), 95 (16), 83 (50), 67 (25), 54 (100), 39 (28), 27 (17).—¹H NMR (400 MHz, CDCl₃): δ (ppm)=1.76-1.78 (m, 12H), 3.55 (dd, J=8.3, 7.5 Hz, 1H), 3.66 (dd, J=7.8, 6.4 Hz, 1H), 4.01 (dd, J=7.8, 7.0 Hz, 1 H), 4.21 (ddd, J=8.3, 6.4, 0.4 Hz, 1H), 4.45-4.51 (m, 1H), 4.49-4.55 (m, 1H), 5-21-5.25 (m, 2H), 5.25-5.30 (m, 2H), 5.33-5.39 (m, 2H), 5.58 (d, J=7.3 Hz, 1H), 5.65 (d, J=7.4 Hz, 1H), 5.81-5.92 (m, 2H).—¹³C NMR (100 MHz, CDCl₃): δ (ppm)=18.4 (2×CH₃), 25.9 (2×CH₃), 69.6 (CH₂), 70.2 (CH₂), 76.9 (CH), 77.8 (CH), 100.4 (CH), 100.9 (CH), 117.7 (CH₂), 118.2 (CH₂), 121.5 (CH), 121.6 (CH), 135.6 (CH), 135.8 (CH), 141.5 (2×C_{quart.}).

### EXAMPLE 19

### Synthesis of 2-methyl-2-(3-methylbutyl)-4-vinyl-[1,3]dioxolane

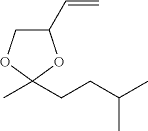

2-methyl-2-(3-methylbutyl)-4-vinyl-[1,3]dioxolane was synthesized by analogy to Example 2, starting with 25.0 g 1-butene-3,4-diol and 23.9 g 5-methyl-2-hexanone. 33.1 g 2-methyl-2-(3-methylbutyl)-4-vinyl-[1,3]dioxolane with a purity of 99% were isolated (2 isomers in the ratio 1:1). This corresponds to a yield of 63% of theoretical.

Boiling point: 35° C./1.0 mbar.—MS: m/z (%)=169 (3), 154 (1), 128 (3), 113 (64), 99 (8), 85 (12), 71 (19), 55 (9), 43 (100), 29 (6).—¹H NMR (400 MHz, CDCl₃): δ (ppm)=0.89 (2 d, J=6.6 Hz, 12H), 1.26-1.33 (m, 4H), 1.34 (s, 3H), 1.37 (s, 3H), 1.49-1.57 (m, 2H), 1.62-1.69 (m, 4 H), 3.55 (t, J=7.9 Hz, 1H), 3.60 (t, J=8.1 Hz, 1H), 4.08-4.12 (m, 2H), 4.43-4.49 (m, 1H), 4.49-4.55 (m, 1H), 5.20-5.24 (m, 2H), 5.32-5.38 (m, 2H), 5.78-5.88 (m, 2H).—¹³C NMR (100 MHz, CDC₃): δ (ppm)=22.5 (CH₃), 22.6 (3×CH₃), 24.1 (CH₃), 24.8 (CH₃), 28.2 (CH), 28.3 (CH), 32.9 (CH₂), 33.0 (CH₂), 37.3 (CH₂), 37.8 (CH₂), 69.3 (CH₂), 69.6 (CH₂), 77.2 (CH), 77.8 (CH), 111.2 (C_{quart.}), 111.3 (C_{quart.}), 118.1 (2×CH₂), 135.6 (CH), 136.0 (CH).

Where in the following examples the compound preferred according to the invention 2-isobutyl-4-vinyl-[1,3]dioxolane has been used, it is a matter in each case of a 1:1 mixture of the cis- and trans-isomers.

### EXAMPLE 20

### Fine Fragrance

| | | Perfume oil A2 |
|---|---|---|
| | Perfume oil A1 | (According to |
| Fragrance substances | (Comparison) | the invention) |
| AMBRAROME 10% in DPG | 20.0 | 20.0 |
| BENZOIN SIAM RESIN 50% in BB | 25.0 | 25.0 |
| BOULEAU ABS. 10% in DPG | 7.0 | 7.0 |
| CEDRENE | 15.0 | 15.0 |
| CISTUS, LABDANUM ABS. | 25.0 | 25.0 |
| 10% in DPG | | |
| COUMARIN | 60.0 | 60.0 |
| DAMASCONE BETA 10% in DPG | 10.0 | 10.0 |
| DIPROPYLENE GLYCOL | 580.0 | 540.0 |
| EUGENOL | 2.0 | 2.0 |
| BETA IONONE | 15.0 | 15.0 |
| ISO E SUPER | 100.0 | 100.0 |
| LINALOOL | 15.0 | 15.0 |
| LINALYL ACETATE | 20.0 | 20.0 |
| METHYL CYCLOPENTENOLONE- | 3.0 | 3.0 |
| 3,2,2, NATURAL, 10% in DPG | | |
| OLIBANUM COEUR 50% in TEC | 30.0 | 30.0 |
| OPOPONAX OIL 10% in DPG | 10.0 | 10.0 |
| SANDALORE | 10.0 | 10.0 |
| LEATHER BASE (SUEDERAL LT) | 3.0 | 3.0 |
| TOLU RESIN 50% in DPG | 15.0 | 15.0 |
| VANILLIN | 20.0 | 20.0 |
| VETIVER OIL JAVA 10% in DPG | 15.0 | 15.0 |
| 2-ISOBUTYL-4-VINYL-[1,3] | | 40.0 |
| DIOXOLANE 10% in DPG | | |
| | 1000.0 | 1000.0 |

Through the addition of 2-isobutyl-4-vinyl-[1,3]dioxolane to the scent composition (at a dosage of 6% of the perfume oil in ethanol) the leathery note is enhanced. 2-isobutyl-4-vinyl-[1,3]dioxolane also provides the composition with greater power and fullness.

### EXAMPLE 21

### Body Lotion

| Component | Wt. % | Wt. % |
|---|---|---|
| Paraffin oil | 5.00 | 5.00 |
| Isopropyl palmitate | 5.00 | 5.00 |
| Cetyl alcohol | 2.00 | 2.00 |
| Beeswax | 2.00 | 2.00 |
| Ceteareth-20 | 2.00 | 2.00 |
| PEG-20-glyceryl stearate | 1.50 | 1.50 |
| Glycerin | 3.00 | 3.00 |
| Phenoxyethanol | 0.50 | — |
| Parabens (mixture of methyl-, ethyl-, | — | 0.50 |
| propyl-, butyl- and isobutylparaben) | | |
| Perfume oil B1 or B2 | 0.50 | 0.50 |
| Water | To 100 | To 100 |

### Perfume Oils for Body Lotion of Example 21

| | | Perfume oil B2 |
|---|---|---|
| | Perfume oil B1 | (According to the |
| Fragrance substances | (Comparison) | invention) |
| AGRUMEX LC | 5.0 | 5.0 |
| ALDEHYDE C14 SO-CALLED | 2.0 | 2.0 |
| AMAROCIT ® | 2.0 | 2.0 |
| AMBERWOODO ® F | 10.0 | 10.0 |
| BENZOIN SIAM RESIN 50% IN BB | 3.0 | 3.0 |
| BERGAMOT SYNTHESSENCE | 40.0 | 40.0 |
| AFRICAN | | |
| CEDAR WOOD OIL VIRGINIA | 30.0 | 30.0 |
| CEDRAMBER | 15.0 | 15.0 |
| CITRONELLOL | 6.0 | 6.0 |
| COUMARIN | 3.0 | 3.0 |
| DIETHYL MALONATE | 8.0 | 8.0 |
| DIHYDROMYRCENOL | 8.0 | 8.0 |
| ETHYL DECADIENOATE TRANS | 10.0 | 10.0 |
| CIS-2,4 10% in IPM | | |
| ETHYL VANILLIN | 4.0 | 4.0 |
| ETHYLENE BRASSYLATE | 120.0 | 120.0 |
| FLOROSA | 30.0 | 30.0 |
| GERANIOL | 5.0 | 5.0 |
| GERANYL ACETATE | 2.0 | 2.0 |
| CHINESE GINGER OIL | 2.0 | 2.0 |
| GIVESCONE 10% in DPG | 6.0 | 6.0 |
| HEDIONE | 65.0 | 65.0 |
| HELIONAL | 30.0 | 30.0 |
| HEXENOL CIS-3 10% in DPG | 10.0 | 10.0 |
| HEXENYL ACETATE CIS-3 | 20.0 | 20.0 |
| 10% in DPG | | |
| HEXYL ACETATE | 3.0 | 3.0 |
| HEXYL SALICYLATE | 65.0 | 65.0 |
| INDOLE 10% in DPG | 3.0 | 3.0 |
| ISO E SUPER | 225.0 | 225.0 |
| LILIAL | 30.0 | 30.0 |
| LINALOOL | 35.0 | 35.0 |
| LINALYL ACETATE | 40.0 | 40.0 |
| METHYL ANTHRANILATE | 2.0 | 2.0 |
| METHYL NAPHTYL KETONE | 3.0 | 3.0 |
| BETA CRIST. | | |
| NEROLI BASE | 6.0 | 6.0 |
| ORANGE OIL BRAZILIAN | 60.0 | 60.0 |
| PRENYL ACETATE 10% in DPG | 10.0 | 10.0 |
| SANDRANOL ® | 10.0 | 10.0 |
| STYRALLYL ACETATE 10% in DPG | 8.0 | 8.0 |
| SYMROXANE ® | 3.0 | 3.0 |
| TETRAHYDROLINALOOL | 50.0 | 50.0 |
| TUBEROSE ABS. 1% in DPG | 1.0 | 1.0 |
| DIPROPYLENE GLYCOL (DPG) | 10.0 | |
| 2-ISOBUTYL-4-VINYL-[1,3] 0.1% in | | 10.0 |
| DPG | | |
| | 1000.0 | 1000.0 |

Through the addition of 2-isobutyl-4-vinyl-[1,3]dioxolane to the scent composition a more natural, stronger, rosy note results. In addition the indolic top note is smoothed and the end note is creamier.

### EXAMPLE 22

### Fabric Softener

| Material | Manufacturer | Chemical name | Function | Wt. % |
|---|---|---|---|---|
| Deionized | | Water | Solvent | 72.4 |
| water | | | | |
| Rewoquat | Evonic | Dialkyl- | Cationic | 16.6 |
| WE 18 | Goldschmidt | esterammonium- | surfactant | |
| | GmbH | ethosulfate | | |
| Mergal K9N | Honeywell | 5-chloro-2-methyl-3- | Preservative | 0.10 |
| | Austria | (2H)-isothiazolone | | |
| | GmbH | and 2-methyl-3- | | |
| | | (2H)-isothiazolone | | |
| Dow Corning | Dow Corning | Polydimethyl- | Defoaming | 0.30 |
| 1520 Antifoam | GmbH, | siloxane | agent | |
| | Deutschland | | | |
| Magnesium | | Magnesium chloride | Consistency | 10.00 |
| chloride 1% | | solution | agent | |
| solution | | | | |
| Fragrance oil | Symrise | | Fragrance | 0.60 |
| C1 or C2 | | | (fragrance) | |

### Perfume Oils for the Fabric Softener of Example 22

| | | Perfume oil |
|---|---|---|
| | Perfume oil | C2 (Accord- |
| | C1 (Com- | ing to the |
| Fragrance substances | parison) | invention) |
| ACETO ACETIC ACID | 15.0 | 15.0 |
| ETHYL ESTER | | |
| ALDEHYDE C14 SO-CALLED | 75.0 | 75.0 |
| ALLYL CAPRONATE | 15.0 | 15.0 |
| BENZALDEHYDE | 5.0 | 5.0 |
| BENZYL ACETATE | 50.0 | 50.0 |
| CASSIS BASE 345B TYPE | 15.0 | 15.0 |
| CITRONELLOL | 30.0 | 30.0 |
| GAMMA DECALACTONE | 60.0 | 60.0 |
| DIPROPYLENE GLYCOL (DPG) | 145.0 | 144.0 |
| ETHYL ACETATE | 5.0 | 5.0 |
| ETHYL BUTYRATE | 5.0 | 5.0 |
| GLOBALIDE ® | 65.0 | 65.0 |
| HEDIONE | 100.0 | 100.0 |
| HERBAFLORAT | 170.0 | 170.0 |
| HEXENOL CIS-3 | 15.0 | 15.0 |
| HEXYL ACETATE | 75.0 | 75.0 |
| IONONE BETA | 5.0 | 5.0 |
| ISOAMYL ACETATE | 3.0 | 3.0 |
| ISORALDEINE 70 | 35.0 | 35.0 |
| LIGUSTRAL | 10.0 | 10.0 |
| LINALOOL | 45.0 | 45.0 |
| METHYL CINNAMATE | 25.0 | 25.0 |
| ROSE OXIDE | 2.0 | 2.0 |
| SANDRANOL ® | 25.0 | 25.0 |
| VANILLIN 10% in DPG | 5.0 | 5.0 |
| 2-ISOBUTYL-4-VINYL-[1,3] | | 1.0 |
| DIOXOLANE 10% in DPG | | |
| | 1000.0 | 1000.0 |

Through the addition of 2-isobutyl-4-vinyl-[1,3]dioxolane to the scent composition a more natural, fruitier note results.

### EXAMPLE 23

### Soap

| Material | Manufacturer | Chemical name | Function | Wt. % |
|---|---|---|---|---|
| Deionized water | | Water | Solvent | 2.0 |
| Soap bases mix | Various | Sodium | Surfactants | 96.95 |
| | | tallowates/ | | |
| | | palmitates | | |
| Titanium | Kronos Titan | Titanium | Colorant/ | 1.0 |
| dioxide | GmbH, | dioxide | lightener | |
| | Deutschland | | | |
| 2-isobutyl- | Symrise | | Perfume | 0.05 |
| 4-vinyl- | | | (fragrance) | |
| [1,3]dioxolane | | | | |

The addition of 2-isobutyl-4-vinyl-[1,3]dioxolane results in a pleasant, natural olives note, reminiscent of “Savon de Marseille”. Furthermore the basic odor of the soap is very well masked by the proportion of 2-isobutyl-4-vinyl-[1,3]dioxolane.

### EXAMPLE 24

### Olive Oil Flavor Concentrate

| | Flavor | Flavor |
|---|---|---|
| | concentrate | concentrate D2 |
| | D2 | (According to the |
| Flavoring substances | (Comparison) | invention) |
| HEXENOL CIS-3 | 2.0 | 2.0 |
| L-ALPHA TERPINEOL CRIST. | 2.0 | 2.0 |
| TRANS-2-DECENAL | 2.0 | 2.0 |
| ETHYL CAPRINATE | 2.0 | 2.0 |
| BUTYRIC ACID | 5.0 | 5.0 |
| ETHYL BUTYRATE | 5.0 | 5.0 |
| CAPRYLIC ACID NATURAL | 5.0 | 5.0 |
| ETHYL LAURATE | 5.0 | 5.0 |
| GUAIACOL | 5.0 | 5.0 |
| ETHYL METHYL BUTYRATE-2 | 7.0 | 7.0 |
| TRANS-2-HEXENAL 10% | 10.0 | 10.0 |
| in TRIACETIN | | |
| ACETIC ACID | 10.0 | 10.0 |
| TRANS, TRANS-2,4-DECADIENAL | 10.0 | 10.0 |
| 10% in VEGETABLE OIL | | |
| PHENYLETHYL ALCOHOL | 20.0 | 20.0 |
| LINALOOL | 10.0 | 10.0 |
| 2-METHYLBUTYRIC ACID | 20.0 | 20.0 |
| 2,5-DIMETHYLPHENOL 10% | 200.0 | 200.0 |
| in ETHANOL | | |
| TRIACETIN | 680.0 | 630.0 |
| 2-ISOBUTYL-4-VINYL- | | 50.0 |
| [1,3]DIOXOLANE | | |
| | 1000.0 | 1000.0 |

The flavor concentrate D2 containing 5% 2-isobutyl-4-vinyl-[1,3]dioxolane had a more authentic, oily, fatty, light green note. This was more reminiscent of fresh cold-pressed olive oil. The flavor concentrate D2 according to the invention was also characterized by more fullness and mouthfill.

### EXAMPLE 25

### Washing Powder

| Material | Manufacturer | Chemical name | Function | Wt. % |
|---|---|---|---|---|
| Sodium | Akzo Nobel | Sodium | | 48.0 |
| metasilicate | Chemicals, | metasilicate | | |
| pentahydrate | Deutschland | pentahydrate | | |
| Sodium hydrogen | Various | Sodium hydrogen | Alkali | 15.0 |
| carbonate | | carbonate | | |
| Sodium carbonate | Various | Sodium carbonate | Bleaching | 15.0 |
| peroxyhydrate | | peroxyhydrate | agent | |
| Peractive AC | Clariant GmbH, | TAED/Na- | Activator | 5.00 |
| Blue | Deutschland | carboxymethyl- | | |
| | | cellulose | | |
| Genapol OA-080 | Clariant GmbH, | Oxoalkohol | Nonionic | 3.00 |
| | Deutschland | C14-15, 8EO | surfactant | |
| Texapon K12 | Cognis | Sodium lauryl | Anionic | 7.00 |
| powder | Deutschland | sulfate C12 | surfactant | |
| | GmbH | | | |
| Tinopal CBS-X | Ciba, | | Lightener | 0.50 |
| | Deutschland | | | |
| Savinase 6.0 T, | Novozymes | Protease | Enzyme | 0.40 |
| Type W | | | | |
| Termamyl 120 T | Novozymes | Alpha-amylase | Enzyme | 0.30 |
| Sodium sulfate | Various | Sodium sulfate | Filler | 5.50 |
| Fragrance oil | Symrise | | Perfume | 0.30 |
| E1 or E2 | | | (fragrance) | |

### Perfume Oils for the Washing Powder of Example 25:

| | Perfume | Perfume oil E2 |
|---|---|---|
| | oil E1 | (According to |
| Fragrance substances | (Comparison) | the invention) |
| ROSEMARY OIL | 5.0 | 5.0 |
| DIHYDROMYRCENOL | 140.0 | 140.0 |
| HERBAFLORAT (ACETIC ACID TRICYCLO[5.2.1.0]- | 50.0 | 50.0 |
| 4-DECEN-8-YL ESTER) | | |
| ALPHA-HEXYL CINNAMALDEHYDE | 125.0 | 125.0 |
| CUMARIN | 10.0 | 10.0 |
| DIPHENYL OXIDE | 5.0 | 5.0 |
| LILIAL ® (2-METHYL-3-(4-TERT.-BUTYLPHENYL)- | 40.0 | 40.0 |
| PROPANAL) | | |
| GALAXOLID ® 50% in DEP (1,1,2,3,3,8-hexamethyl- | 150.0 | 150.0 |
| 1,2,3,5,7,8-hexahydro-6-oxa- | | |
| cyclopenta[b]naphthalene) | | |
| ISORALDEIN ® 70 ((E)-3-methyl-4-(2,6,6-trimethyl- | 30.0 | 30.0 |
| cyclohex-2-enyl)-but-3-en-2-one) | | |
| ALLYL AMYL GLYCOLATE | 7.0 | 7.0 |
| ISO E SUPER ® (3-ACETYL-3,4,10,10- | 80.0 | 80.0 |
| TETRAMETHYL-1(6)-BICYCLO[4.4.0]DECENE) | | |
| ROSE BASE | 100.0 | 100.0 |
| GALBANUM BASE | 15.0 | 15.0 |
| DIPROPYLENE GLYCOL (DPG) | 5.0 | 4.9 |
| APPLE BASE | 25.0 | 25.0 |
| OZONIL 10% in DPG (1Z-DODECENYL CYANIDE) | 15.0 | 15.0 |
| MELONAL ® 10% in DPG (2,6-DIMETHYL-5- | 3.0 | 3.0 |
| HEPTENAL) | | |
| METHYL OCTIN CARBONATE 10% in DPG | 5.0 | 5.0 |
| DELTA DAMASCON 10% in DPG (1-(2,6,6-trimethyl- | 7.0 | 7.0 |
| cyclohex-3-enyl)-but-2-en-1-one) | | |
| SANDRANOL ® (2-ETHYL-4-(2,2,3-TRIMETHYL-3- | 20.0 | 20.0 |
| CYCLOPENTENYL)-2E-BUTENOL) | | |
| VERTOCITRAL (TRANS-2,4-DIMETHYL-1-FORMYL- | 3.0 | 3.0 |
| 3-CYCLOHEXENE) | | |
| AGRUMEX (ACETIC ACID-2-TERT.- | 100.0 | 100.0 |
| BUTYLCYCLOHEXYL ESTER) | | |
| BENZYL ACETONE | 10.0 | 10.0 |
| ALDEHYDE C12 MNA (methyl nonyl acetaldehyde) | 5.0 | 5.0 |
| PROJASMON P (2-HEPTYL-CYCLOPENTANONE) | 10.0 | 10.0 |
| NEROLIN YARA YARA | 15.0 | 15.0 |
| INTRELEVEN ALDEHYDE (10-undecenal) 10% in | 5.0 | 5.0 |
| DPG | | |
| PATCHOULI OIL | 15.0 | 15.0 |
| 2-ISOBUTYL-4-VINYL-[1,3]DIOXOLANE | | 0.1 |
| | 1000.0 | 1000.0 |

At a dosing of 0.3% of perfume oil E1 or E2 in a washing powder (powder detergent) the following finding was made: the amount of 2-isobutyl-4-vinyl-[1,3]dioxolane in perfume oil E2 brings about an intensification of the green notes. Overall, the composition with 2-isobutyl-4-vinyl-[1,3]dioxolane also radiates more freshness.

### EXAMPLE 26

### All-Purpose Cleaner

| Material | Manufacturer | Chemical name | Function | Wt. % |
|---|---|---|---|---|
| Deionized | | Water | Solvent | 59.6 |
| water | | | | |
| Mergal K9N | Troy Chemie, | 5-chloro-2-methyl- | Preservative | 0.1 |
| | Seelze | 3-(2H)-isothiazolone | | |
| | | and 2-methyl-3- | | |
| | | (2H)-isothiazolone | | |
| Trisodium | Various | Trisodium citrate | Complexing | 3.0 |
| citrate | | dihydrate | agent | |
| dihydrate | | | | |
| Zetesol | Zschimmer & | Fatty alcohol C12- | Anionic | 30.0 |
| NL-2 | Schwarz, | 14-sulfate, sodium | surfactant | |
| | Deutschland | | | |
| Imbentin | Dr. W. Kolb | Fatty alcohol | Non-ionic | 5.0 |
| C/125/055 | AG Chem. | C12-C15, 8EO | surfactant | |
| Ethanol 96% | Various | Ethanol | Solvent | 2.0 |
| Fragrance | Symrise | | Perfume | 0.3 |
| oil F1 or F2 | | | (fragrance) | |

Perfume Oils for the all-Purpose Cleaner of Example 26:

| | Perfume | Perfume oil F2 |
|---|---|---|
| | oil F1 | (According to the |
| Fragrance substances | (Comparison) | invention) |
| CEDAR WOOD OIL | 8.0 | 8.0 |
| AMBROCENID ® 10% in DPG | 1.0 | 1.0 |
| ROSEMARY OIL | 8.0 | 8.0 |
| DIHYDROMYRCENOL | 80.0 | 80.0 |
| ISOBUTYLQUINOLINE | 0.5 | 0.5 |
| AMBROXAN | 1.0 | 1.0 |
| LIGUSTRAL (TRANS-2,4-DIMETHYL-1-FORMYL- | 2.0 | 2.0 |
| 3-CYCLOHEXENE) | | |
| AMYL SALICYLATE N/ISO | 24.0 | 24.0 |
| CITRONELLOL | 8.0 | 8.0 |
| VERTOFIX (1-(3,6,8,8-tetramethyl-2,3,4,7,8,8a- | 30.0 | 30.0 |
| hexahydro-1H-3a,7-methano-azulen-5-yl)- | | |
| ethanone) | | |
| ALPHA-HEXYL CINNAMALDEHYDE | 50.0 | 50.0 |
| CUMARIN | 4.0 | 4.0 |
| COUMARONE (2-ACETYL-BENZOFURAN) | 0.6 | 0.6 |
| ISOBORNYL ACETATE | 30.0 | 30.0 |
| CAMPHOR | 8.0 | 8.0 |
| LILIAL ® (2-METHYL-3-(4-TERT.- | 30.0 | 30.0 |
| BUTYLPHENYL)-PROPANAL) | | |
| LINALOOL | 40.0 | 40.0 |
| LINALYL ACETATE | 40.0 | 40.0 |
| TERPINEOL | 100.0 | 100.0 |
| GALAXOLID ® 50% in DEP (1,1,2,3,3,8- | 24.0 | 24.0 |
| hexamethyl-1,2,3,5,7,8-hexahydro-6-oxa- | | |
| cyclopenta[b]naphthalene) | | |
| GLOBALIDE ® (PENTADECEN-1,15-OLIDE, | 5.0 | 5.0 |
| 11E/Z) | | |
| ETHYLENE BRASSYLATE (BRASSYLIC ACID | 24.0 | 24.0 |
| ETHANE DIOL ESTER) | | |
| AGRUNITRIL (2,6-DIMETHYL-5- | 16.0 | 16.0 |
| HEPTENYLCYANIDE) | | |
| ISORALDEIN ® 70 (E)-3-methyl-4-(2,6,6-trimethyl- | 20.0 | 20.0 |
| cyclohex-2-enyl)-but-3-en-2-one) | | |
| OZONIL (1Z-DODECENYLCYANIDE) | 1.5 | 1.5 |
| ALLYL AMYL GLYCOLATE (2-METHYLBUTOXY- | 15.0 | 15.0 |
| ACETIC ACID ALLYL ESTER) | | |
| DAMASCONE DELTA ((1-(2,6,6-trimethyl- | 2.0 | 2.0 |
| cyclohex-3-enyl)-but-2- en-1-one) | | |
| TIMBEROL ® (2,2,6-TRIMETHYL-1-(3- | 2.0 | 2.0 |
| HYDROXYHEXYL)-CYCLOHEXANE) | | |
| ESTRAGOL | 1.0 | 1.0 |
| MYSORANE ® BASE | 10.0 | 10.0 |
| DIPROPYLENE GLYCOL | 346.2 | 346.15 |
| LEMON OIL TERPENE | 20.0 | 20.0 |
| GERANIOL | 8.0 | 8.0 |
| GERANIUM BASE | 5.0 | 5.0 |
| PATCHOULI BASE | 8.0 | 8.0 |
| GALBASCONE 10% in DPG (4,4-DIMETHYL-2-(1- | 2.0 | 2.0 |
| OXO-4-PENTENYL)-CYCLOHEXENE) | | |
| CALONE ® 1951 10% in DPG (7-METHYL-3,4- | 4.0 | 4.0 |
| DIHYDRO-3-BENZODIOXEPINONE | | |
| ISOBUTYL QUINOLINE | 0.2 | 0.2 |
| EUGENOL | 1.5 | 1.5 |
| FARENAL ® ((2,6,10-TRIMETHYL-9- | 0.5 | 0.5 |
| UNDECENAL) | | |
| LIME OIL | 12.0 | 12.0 |
| ANETHOLE | 4.0 | 4.0 |
| BETA METHYL NAPHTHYL KETONE | 3.0 | 3.0 |
| 2-ISOBUTYL-4-VINYL-[1,3]DIOXOLANE | | 0.05 |
| | 1000.0 | 1000.0 |

At a dosing of 0.3% of the perfume oil in the all-purpose cleaner, the following finding was made: the amount of 2-isobutyl-4-vinyl-[1,3]dioxolane in perfume oil F2 brings about an intensification of the woody-mossy note. In addition, the diffusivity is increased.

### EXAMPLE 27

### Shampoo

| Material | Manufacturer | INCI-Name | Wt. % |
|---|---|---|---|
| Deionized water | | Water | 71.5 |
| Plantacare PS 10 | Cognis | Sodium laureth sulfate, | 20.0 |
| | Deutschland | | |
| | GmbH | Lauryl glucoside | |
| Euperlan PK 771 | Cognis | Glycol distearate, | 6.0 |
| | Deutschland | | |
| | GmbH | Sodium lauryl | |
| | | sulfate, Cocamide | |
| | | MEA, Laureth-10 | |
| Dragocid Liquid | Symrise | Phenoxyethanol, | 0.5 |
| | | Methylparaben, | |
| | | Ethylparaben, | |
| | | Butylparaben, | |
| | | Propylparaben, | |
| | | Isobutylparaben | |
| Sodium chloride | | Sodium chloride | 1.4 |
| Citric acid | | Citric acid | 0.1 |
| monohydrate | | | |
| crystalline | | | |
| Fragrance oil | Symrise | Perfume (Fragrance) | 0.5 |
| G1 or G2 | | | |

### Perfume Oils for the Shampoos of Example 27:

| | Perfume | Perfume oil G2 |
|---|---|---|
| | oil G1 | (According to the |
| Fragrance substances | (Comparison) | invention) |
| CLOVE BUD OIL | 10.0 | 10.0 |
| PATCHOULI OIL | 79.5 | 79.5 |
| DIHYDROMYRCENOL | 60.0 | 60.0 |
| ISORALDEINE ® ((E)-3-methyl-4-(2,6,6-trimethyl- | 20.0 | 20.0 |
| cyclohex-2-enyl)-but-3-en-2-one) | | |
| EBANOL (3-METHYL-5-(2,2,3-TRIMETHYL-3- | 15.0 | 15.0 |
| CYCLOPENTENYL)-4E/Z-PENTEN-2-ONE) | | |
| GLOBALIDE ® (E/Z-11-PENTADECEN-1,15-GLIDE) | 60.0 | 60.0 |
| ALPHA-HEXYL CINNAMALDEHYDE | 40.0 | 40.0 |
| COUMARIN | 20.0 | 20.0 |
| LILIAL ® (2-METHYL-3-(4-TERT.-BUTYLPHENYL)- | 30.0 | 30.0 |
| PROPANAL) | | |
| LINALOOL | 20.0 | 20.0 |
| LINALYL ACETATE | 30.0 | 30.0 |
| VANILLIN | 20.0 | 20.0 |
| LYRAL ® (4-FORMYL-2-(4-40.0 40.0 HYDROXY-4- | 40.0 | 40.0 |
| METHYL-PENTYL)-CYCLOHEXENE) | | |
| HEDION ® (METHYL-CIS/TRANS- | 30.0 | 30.0 |
| DIHYDROJASMONATE) | | |
| EVERNYL ® (2,4-DIHYDROXY-3,6-DIMETHYL- | 5.0 | 5.0 |
| BENZOIC ACID METHYL ESTER) | | |
| CEDRAMBER (CEDRYL METHYL ETHER) | 20.0 | 20.0 |
| ISO E SUPER ® (3-ACETYL-3,4,10,10- | 150.5 | 150.0 |
| TETRAMETHYL-1(6)-BICYCLO[4.4.0]DECENE) | | |
| GERANIUM BASE | 20.0 | 20.0 |
| BERGAMOT BASE | 80.0 | 80.0 |
| WORMWOOD OIL | 10.0 | 10.0 |
| GALBANUM OIL 10% in DPG | 15.0 | 15.0 |
| AMBROCENIDE ® 0.1% in DPG | 20.0 | 20.0 |
| CYCLOGALBANAT ® 10% in DPG | 5.0 | 5.0 |
| (CYCLOHEXYLOXYACETIC ACID ALLYL ESTER) | | |
| CISTUS OIL 10% in DPG | 10.0 | 10.0 |
| SPEARMINT OIL 10% in DPG | 10.0 | 10.0 |
| AURELIONE (E/Z-7/8-CYCLOHEXADECENONE) | 120.0 | 120.0 |
| AMBROXIDE | 5.0 | 5.0 |
| MANDARIN OIL | 5.0 | 5.0 |
| LAVANDIN OIL GROSSO | 30.0 | 30.0 |
| LEMON OIL | 20.0 | 20.0 |
| 2-ISOBUTYL-4-VINYL-[1,3]DIOXOLANE | | 0.5 |
| | 1000.0 | 1000.0 |

At a dosage of 0.5% of the perfume oil in the shampoo the following finding was made: the addition of 2-isobutyl-4-vinyl-[1,3]dioxolane in perfume oil G2 intensifies the fruity character. The composition also appears to be much creamier and rounder.

### EXAMPLE 28

### Shower Gel

| Material | Manufacturer | INCI-Name | Wt. % |
|---|---|---|---|
| Deionized water | | Water | 76.3 |
| Plantacare PS 10 | Cognis | Sodium laureth sulfate, | 20.0 |
| | Deutschland | Lauryl glucoside | |
| | GmbH | | |
| Dragocid Liquid | Symrise | Phenoxyethanol, | 0.5 |
| | | Methylparaben, | |
| | | Ethylparaben, | |
| | | Butylparaben, | |
| | | Propylparaben, | |
| | | Isobutylparaben | |
| Sodium chloride | | Sodium chloride | 1.4 |
| Citric acid Citric | | Citric acid | 1.3 |
| acid monohydrate | | | |
| crystalline | | | |
| Fragrance oil | Symrise | Perfume (Fragrance) | 0.5 |
| H1 or H2 | | | |

### Perfume Oil for the Shower Gels of Example 28:

| | Perfume | Perfume oil H2 |
|---|---|---|
| | oil H1 | (According to the |
| Fragrance substance(s) | (Comparison) | invention) |
| CASSIS BASE | 10.0 | 10.0 |
| CLOVE BUD OIL | 5.0 | 5.0 |
| PATCHOULI OIL | 5.0 | 5.0 |
| DIHYDROMYRCENOL | 60.0 | 60.0 |
| HEXYL SALICYLATE | 20.0 | 20.0 |
| HEDION ® (METHYL-CIS/TRANS- | 210.0 | 210.0 |
| DIHYDROJASMONATE) | | |
| ORANGE OIL | 25.0 | 25.0 |
| GLOBALIDE ® (E/Z-11-PENTADECEN-1,15-OLIDE) | 20.0 | 20.0 |
| POLYSANTOL ® (3,3-DIMETHYL-(2,2,3-TRIMETHYL- | 5.0 | 5.0 |
| 3-CYCLOPENTENYL)-4-PENTEN-2-OL) | | |
| LAVANDIN OIL GROSSO | 20.0 | 20.0 |
| YSAMBER ® K (ISOLONGIFOLANONE | 20.0 | 20.0 |
| ETHANEDIOL KETAL) | | |
| ALPHA-HEXYL CINNAMALDEHYDE | 100.0 | 100.0 |
| LILIA ® (2-METHYL-3-(4-TERT.-BUTYLPHENYL)- | 50.0 | 50.0 |
| PROPANAL) | | |
| LINALOOL | 60.0 | 60.0 |
| LINALYL ACETATE | 50.0 | 50.0 |
| TERPINEOL | 20.0 | 20.0 |
| ETHYLENE BRASSYLATE (BRASSYLIC ACID | 20.0 | 20.0 |
| ETHANEDIOL ESTER) | | |
| ALLYL AMYL GLYCOLATE (2-METHYLBUTOXY- | 10.0 | 10.0 |
| ACETIC ACID ALLYL ESTER) | | |
| ISO E SUPER ® (3-ACETYL-3,4,10,10- | 50.0 | 50.0 |
| TETRAMETHYL-1(6)-BICYCLO[4.4.0]DECENE) | | |
| KEPHALIS (3,3,5,5-TETRAMETHYL-4-(1- | 5.0 | 5.0 |
| ETHOXYVINYL)-CYCLOHEXANONE) | | |
| BERGAMOT BASE | 100.0 | 100.0 |
| FLORALOZONE (2-METHYL-2-(4-ETHYLBENZYL)- | 10.0 | 10.0 |
| PROPANAL) | | |
| MANDARIN ALDEHYDE 10% in TEC (2E- | 5.0 | 5.0 |
| DODECENAL) | | |
| LIGUSTRAL ® 10% in DPG (TRANS-2,4-DIMETHYL- | 10.0 | 10.0 |
| 1-FORMYL-3-CYCLOHEXENE) | | |
| ALPHA-DAMASCONE 1% in DPG ((E/Z)-1-(2,6,6- | 20.0 | 20.0 |
| trimethyl-cyclohex-2-enyl)-but-2-en-1-one) | | |
| FARENAL ® 1% in DPG (2,6,10-TRIMETHYL-9- | 20.0 | 20.0 |
| UNDECENAL) | | |
| LEAFOVERT ® 10% in DPG (CARBONIC ACID-3Z- | 20.0 | 20.0 |
| HEXENYL METHYL ESTER) | | |
| CALONE ® 1951 10% in DPG (7-METHYL-3,4- | 30.0 | 30.0 |
| DIHYDRO-3-BENZODIOXEPINONE) | | |
| DIPROPYLENE GLYCOL | 20.0 | 19.8 |
| 2-ISOBUTYL-4-VINYL-[1,3]DIOXOLANE | | 0.2 |
| | 1000.0 | 1000.0 |

At a dosing of 0.5% of the perfume oil in the shower gel, the following finding is made: the addition of 2-isobutyl-4-vinyl-[1,3]dioxolane in perfume oil H2 gives the composition a more radiating aromatic note.

### EXAMPLE 29

### Transparent Deodorant Sticks (Formulations A and B) or Deodorant Crème Sticks (Formulations C and D)

| | A | B | C | D |
|---|---|---|---|---|
| Components | Wt. % | Wt. % | Wt. % | Wt. % |
| Aluminum zirconium | 25.00 | 20.00 | 25.00 | 20.00 |
| tetrachlorohydrate-glycine complex | | | | |
| Dimethicone (10 Cst) | — | — | 5.00 | 5.00 |
| Cyclopentasiloxane | — | 0.50 | 1.00 | 0.50 |
| Petrolatum | 5.00 | 4.70 | 5.00 | 5.00 |
| Ozocerite | 1.00 | 1.50 | — | — |
| Stearyl alcohol | 12.00 | 12.00 | — | — |
| 2-butyloctanic acid | 0.50 | — | 0.50 | — |
| Wax | — | — | 1.25 | 1.25 |
| PPG-14 butyl ether | 9.00 | 9.00 | — | — |
| Hardened rapeseed oil | — | — | 5.00 | 5.00 |
| Silicon dioxide | — | — | 1.00 | — |
| Farnesol | 0.25 | — | 0.25 | — |
| Paraffin oil | 0.50 | 0.50 | — | — |
| Hydrogenated castor oil (castor wax) | 3.50 | 3.50 | — | — |
| Talc | 4.00 | 4.00 | — | — |
| Behenyl alcohol | 0.20 | 0.20 | — | — |
| d-Panthenyl triacetate | 1.00 | 1.00 | — | — |
| Preservative | q.s. | q.s. | q.s. | q.s. |
| Perfume oil G2 from Example 27 | 1.50 | — | 1.15 | — |
| Perfume oil H2 from Example 28 | | 0.90 | — | 0.75 |
| Water | To 100 | To 100 | To 100 | To 100 |

### EXAMPLE 30

### Antiperspirant Roll-on

| Components | Wt. % | Wt. % |
|---|---|---|
| Caprylyl trimethicone (SilCare ™ silicone 31 M 50) | 0.30 | 0.30 |
| Steareth-20 (GENAPOL ™ HS 200) | 3.00 | 3.00 |
| Steareth-2 (GENAPOL ™ HS 020) | 1.50 | 1.50 |
| Dicaprylyl ether (Cetiol ™ OE) | 2.00 | 2.00 |
| Coco-caprylate/caprate (Cetiol ™ LC) | 2.00 | 2.00 |
| Glycerin | 2.00 | 2.00 |
| Glyceryl stearate (Cutina ™ GMS) | 2.00 | 2.00 |
| Octyl dodecanol (Eutanol ™ G) | 1.00 | 1.00 |
| Stearyl alcohol | 2.50 | 2.50 |
| Aluminum chlorohydrate according to Example 1 of EP | 10.00 | 10.00 |
| 1321431 | | |
| Avocado extract Persea gratissima | 0.30 | 0.20 |
| Perfume oil G2 from Example 27 | 0.50 | — |
| Perfume oil H2 from Example 28 | — | 0.60 |
| Water | To 100 | To 100 |

### EXAMPLE 31

### Antiperspirant Stick

| Components | Wt. % | Wt. % |
|---|---|---|
| Phenyl trimethicone (SilCare ™ Silicone 15 M 50) | 13.50 | 13.50 |
| Cetearyl alcohol | To 100 | To 100 |
| Cetiol CC (dicaprylyl carbonate) | 13.50 | 13.50 |
| Stearic acid | 3.50 | 3.50 |
| PEG-40 hydrogenated castor oil (Emulsogen ™ HCO | 4.10 | 4.10 |
| 040) | | |
| PEG-8 distearate (Cithrol 4 DS) | 4.10 | 4.10 |
| Petrolatum | 6.90 | 6.90 |
| Aluminum chlorohydrate | 13.80 | 13.80 |
| Aluminum zirconium trichlorohydrex Gly | 19.50 | 20.00 |
| Ethylhexyl glycerine (octoxy glycerine) | 0.30 | 0.20 |
| 4-methyl-4-phenyl-2-pentanol (Vetikol) | 0.25 | 0.10 |
| Perfume oil A2 from Example 20 | 1.00 | — |
| Perfume oil G2 from Example 27 | — | 0.80 |

### EXAMPLE 32

### Aerosol Spray

| Components | Wt. % | Wt. % | Wt. % |
|---|---|---|---|
| Octyldodecanol | 0.50 | — | 0.50 |
| Phenoxyethanol | — | — | 0.30 |
| 1,2-pentanediol | 1.00 | 1.00 | 0.50 |
| 1,2-hexanediol | 0.25 | 0.15 | 0.25 |
| 1,2-octanediol | 0.25 | 0.25 | 0.25 |
| Farnesol | — | 0.25 | 0.15 |
| Ethylhexyl glycerine (octoxy glycerine) | 0.50 | 0.30 | 0.50 |
| Perfume oil A2 from Example 20 | 0.80 | — | 0.50 |
| Perfume oil H2 from Example 28 | — | 1.15 | 0.50 |
| Ethanol | To 100 | To 100 | To 100 |

The mixture obtained after mixing together each of the components indicated was filled with a propane-butane mixture (2:7) in a weight ratio of 2:3 in an aerosol container.

### EXAMPLE 33

### Hair Conditioner with UV Protection

| Components | INCI Name | Wt. % | Wt. % |
|---|---|---|---|
| Lanette O | Cetearyl alcohol | 4.00 | 4.00 |
| Dragoxat 89 | Ethylhexyl isononanoate | 4.00 | 4.00 |
| Emulsiphos | Potassium cetyl phosphate, | 0.50 | 0.50 |
| | Hydrogenated palm glycerides | | |
| Natrosol 250 HR | Hydroxyethylcellulose | 0.25 | 0.25 |
| Neo Heliopan Hydro | Phenylbenzimidazole sulfonic acid | 2.00 | 2.00 |
| L- Arginin | Arginine | 1.20 | 1.20 |
| Benzophenon-4 | Benzophenone-4 | 0.50 | 0.50 |
| Neo Heliopan AP | Disodium phenyl dibenzimidazole | 0.50 | 1.00 |
| | tetrasulfonate | | |
| Edeta BD | Disodium EDTA | 0.05 | 0.05 |
| Dragocide Liquid | Phenoxyethanol (and) methylparaben | 0.80 | 0.80 |
| | (and) butyparaben (and) ethyparaben | | |
| | (and) propylparaben | | |
| Dow Corning 949 Cationic | Amodimethicone, Cetrimonium | 2.00 | 2.00 |
| Emulsion | Chloride, Trideceth-12 | | |
| Dow Corning 5200 | Laurylmethicone copolyol | 0.50 | 0.50 |
| Perfume oil B2 from Example 21 | Fragrance | 0.95 | — |
| Perfume oil H2 from Example 28 | Fragrance | | 1.25 |
| Water | Water (Aqua) | To 100 | To 100 |

### EXAMPLE 34

### Sun Protection Spray

| Part | Raw materials | INCI name | Wt. % |
|---|---|---|---|
| A | Water, demineralized | Water (Aqua) | 69.50 |
| | Glycerin | Glycerin | 4.00 |
| | 1,3 butylene glycol | Butylene glycol | 5.00 |
| | D-Panthenol | Panthenol | 0.50 |
| | Lara Care A-200 | Galactoarabinan | 0.25 |
| B | Baysilone oil M 10 | Dimethicone | 1.00 |
| | Edeta BD | Disodium EDTA | 0.10 |
| | Copherol 1250 | Tocopheryl acetate | 0.50 |
| | Cetiol OE | Dicaprylyl ether | 3.00 |
| | Neo Heliopan ® HMS | Homosalate | 5.00 |
| | Neo Heliopan ® AV | Ethylhexyl methoxycinnamate | 6.00 |
| | Neo Heliopan ® 357 | Butyl methoxydibenzoylmethane | 1.00 |
| | Corapan TQ | Diethylhexylnaphthalate | 2.00 |
| | Alpha Bisabolol | Bisabolol | 0.10 |
| | Pemulen TR-2 | Acrylates/C10-30 alkyl acrylate | 0.25 |
| | | crosspolymer | |
| C | Phenoxyethanol | Phenoxyethanol | 0.70 |
| | Solbrol M | Methylparaben | 0.20 |
| | Solbrol P | Propylparaben | 0.10 |
| D | NaOH, 10% | Sodium Hydroxide | 0.60 |
| E | Perfume oil B2 from | Fragrance (perfume) | 0.20 |
| | Example 21 | | |

Production method
Part A: Dissolve the Lara Care A-200 in the other components while stirring.
Part B: Weigh in all the raw materials (except the Pemulen) and dissolve the crystalline substances by heating. Disperse the Pemulen. Add part B to part A and homogenize for 1 minute.
Parts C-E: add and homogenize for a further 1-2 minutes with the Ultra Turrax.

### EXAMPLE 35

### Sun Protection Soft Cream (W/O), Sun Protection Factor SPF 40

| Part | Raw materials | INCI name | Wt. % |
|---|---|---|---|
| A | Dehymuls PGPH | Polyglyceryl-2 | 5.00 |
| | | dipolyhydroxystearate | |
| | Copherol 1250 | Tocopheryl acetate | 0.50 |
| | Permulgin 3220 | Ozokerite | 0.50 |
| | Zinc stearate | Zinc stearate | 0.50 |
| | Tegosoft TN | C12-15 alkyl benzoate | 10.00 |
| | Neo Heliopan ® E1000 | Isoamyl-p-methoxycinnamate | 2.00 |
| | Neo Heliopan ® 303 | Octocrylene | 5.00 |
| | Neo Heliopan ® MBC | 4-methylbenzylidene camphor | 3.00 |
| | Zinc oxide neutral | Zinc oxide | 5.00 |
| B | Water, distilled | Water (Aqua) | To 100 |
| | EDETA BD | Disodium EDTA | 0.10 |
| | Glycerin | Glycerin | 4.00 |
| | Phenoxyethanol | Phenoxyethanol | 0.70 |
| | Solbrol M | Methylparaben | 0.20 |
| | Solbrol P | Propylparaben | 0.10 |
| | Magnesium sulfate | Magnesium sulfate | 0.50 |
| C | Perfume oil B2 from | Perfume (Fragrance) | 0.30 |
| | Example 21 | | |

Production method
Part A: Heat to approximately 85° C.
Part B: Heat to approximately 85° C. (excluding zinc oxide; disperse the zinc oxide with the Ultra Turrax). Add B to A. Allow to cool while stirring.
Part C: Add and then homogenize.

### EXAMPLE 36

### Sun Protection Milk (W/O)

| Part | Raw materials | INCI name | Wt. % |
|---|---|---|---|
| A | Dehymuls PGPH | Polyglyceryl-2 | 3.00 |
| | | dipolyhydroxystearate | |
| | Beeswax 8100 | Beeswax | 1.00 |
| | Monomuls 90-0-18 | Glyceryl oleate | 1.00 |
| | Zinc stearate | Zinc stearate | 1.00 |
| | Cetiol SN | Cetearyl isononanoate | 5.00 |
| | Cetiol OE | Dicaprylyl ether | 5.00 |
| | Tegosoft TN | C12-15 alkyl benzoate | 4.00 |
| | Vitamin E | Tocopherol | 0.50 |
| | Solbrol P | Propylparaben | 0.10 |
| | Neo Heliopan ® OS | Ethylhexyl salicylate | 5.00 |
| | Neo Heliopan ® AV | Ethylhexyl methoxycinnamate | 7.50 |
| | Uvinul ® T150 | Ethylhexyl triazone | 1.50 |
| B | Water, distilled | Water (Aqua) | To 100 |
| | Trilon BD | Disodium EDTA | 0.10 |
| | Glycerin | Glycerin | 5.00 |
| | Solbrol M | Methylparaben | 0.20 |
| | Phenoxyethanol | Phenoxyethanol | 0.70 |
| | Neo Heliopan ® AP 10% | Disodium phenyl dibenzimidazole | 15.00 |
| | solution, neutralized with | tetrasulfonate | |
| | NaOH | | |
| C | Perfume oil G2 from | Perfume (Fragrance) | 0.25 |
| | Example 27 | | |
| | Alpha Bisabolol | Bisabolol | 0.10 |

Production method
Part A: Heat to approximately 85° C.
Part B: Heat to approximately 85° C. Add B to A. Allow to cool while stirring.
Part C: Add and then homogenize.

### EXAMPLE 37

### Production of a Flavoring of the Eucalyptus-Menthol Type Using 2-Isobutyl-4-Vinyl-[1,3]Dioxolane

| Components | Wt. % |
|---|---|
| Anethole | 10 |
| Peppermint oil, Mentha piperita, Willamette | 20 |
| type | |
| Peppermint oil, Mentha arvensis, rectified | 20 |
| 1,8-cineol (Eucalyptol) | 5 |
| l-menthol | 44 |
| 2-isobutyl-4-vinyl-[1,3]dioxolane | 1 |
| Total | 100 |

The flavoring obtained in this way was incorporated into a standard toothpaste mass with a silica base in a concentration of 1.2 Wt. %. The toothpaste was tested by a panel of sensorially trained experts under practical conditions. The taste of the toothpaste according to the invention was assessed as fuller and more pleasant than that of a toothpaste with a corresponding flavoring without 2-isobutyl-4-vinyl-[1,3]dioxolane.

### EXAMPLE 38

### Production of a Flavoring of the Spearmint Type Using 2-Isobutyl-4-Vinyl-[1,3]Dioxolane.

| Components | Wt. % |
|---|---|
| l-menthol | 29 |
| l-carvone | 20 |
| Spearmint, native type | 20 |
| Anethole | 5 |
| Peppermint oil, Mentha arvensis, rectified | 10 |
| Peppermint oil, Mentha piperita, Willamette | 15 |
| type | |
| 2-isobutyl-4-vinyl-[1,3]dioxolane | 1 |
| Total | 100 |

The flavoring obtained in this way was incorporated into a toothpaste mass in a concentration of 1.2 Wt. %, wherein the toothpaste mass consisted of 65 Wt. % of sodium bicarbonate. The resultant toothpaste was tested by a panel of sensorially trained experts under practical conditions. The flavor of this toothpaste was assessed as a more pleasant, stronger spearmint taste coupled with a voluminous fresh taste.

### EXAMPLE 39

Production of a Flavoring with a Spicy-Aromatic Taste Using 2-Isobutyl-4-Vinyl-[1,3]Dioxolane.

| Components | Wt. % |
|---|---|
| l-menthol | 29 |
| Peppermint oil, Mentha arvensis, rectified | 25 |
| Peppermint oil, Willamette type | 15 |
| Anethole | 10 |
| Spearmint, native type | 10 |
| Cinnamaldehyde | 5 |
| Eugenol | 5 |
| 2-isobutyl-4-vinyl-[1,3]dioxolane | 1 |
| Total | 100 |

This flavoring was incorporated into a standard toothpaste mass with a silica base in a concentration of 1.2 Wt. %. The toothpaste was tested by a panel of sensorially trained experts under practical conditions. The sensorial assessment of the toothpaste was of a very pleasant, full, minty, fruity-fresh taste.

### EXAMPLE 40

Production of a Flavoring with a Wintergreen Taste Using 2-Isobutyl-4-Vinyl-[1,3]Dioxolane.

| Components | Wt. % |
|---|---|
| Anethole | 10 |
| Peppermint oil, Mentha arvensis | 12 |
| Peppermint oil, Mentha piperita, Willamette | 12 |
| type | |
| Methyl salicylate | 25 |
| l-menthol | 40 |
| 2-isobutyl-4-vinyl-[1,3]dioxolane | 1 |
| Total | 100 |

The flavoring obtained in this way was incorporated into a standard toothpaste mass with a silica base in a concentration of 1.2 Wt. %. The toothpaste was tested by a panel of sensorially trained experts under practical conditions. The taste of this toothpaste according to the invention was assessed as fuller and slightly sweeter than that of a toothpaste with a corresponding flavoring without 2-isobutyl-4-vinyl-[1,3]dioxolane.

### EXAMPLE 41

Production of a Flavoring with a Peppermint Taste Using 2-Isobutyl-4-Vinyl-[1,3]Dioxolane.

| Components | Wt. % |
|---|---|
| Anethole | 9 |
| Peppermint oil, Mentha arvensis | 50 |
| l-menthone | 20 |
| l-menthol | 20 |
| 2-isobutyl-4-vinyl-[1,3]dioxolane | 1 |
| Total | 100 |

The flavoring obtained in this way was incorporated into a sugar-free standard chewing gum mass in a concentration of 1.5 Wt. %. The chewing gum was assessed by a panel of trained experts for its sensorial quality. The flavoring demonstrated a pronounced fruity-fresh note, wherein in addition the peppermint taste was intensified. The flavoring further ensured a full taste impression of the chewing gum.

### EXAMPLE 42

### Production of a Mouthwash Flavoring Using 2-Isobutyl-4-Vinyl-[1,3]Dioxolane.

| Components | Wt. % |
|---|---|
| Anethole | 30 |
| Eucalyptol | 25 |
| l-menthol | 44 |
| 2-isobutyl-4-vinyl-[1,3]dioxolane | 1 |
| Total | 100 |

The flavoring was incorporated in a concentration of 0.15 Wt. % into a ready-to-use mouthwash or in a concentration of 3 Wt. % in a mouthwash concentrate. The sensorial assessment by a panel of trained experts in each case showed that the flavoring, as a result of the presence of 2-isobutyl-4-vinyl-[1,3]dioxolane, has a full, very pleasant fruity-fresh note.

The flavoring compositions used in the following examples 43 through 58 are suitable for use in a whole range of different items, wherein the use is not restricted to items such as toothpastes, chewing gums or hardboiled candies. In all the following examples in particular a fuller and more pleasant taste was perceptible.

### EXAMPLE 43

### Toothpaste (‘Silica Opaque’)

All particulars in Wt. % unless otherwise stated.

| Component | A | B |
|---|---|---|
| Deionized water | 26.53 | 26.53 |
| Sorbitol 70% | 45 | To 100 |
| Solbrol M Na-salt | 0.1 | 50.15 |
| Trisodium phosphate | 0.1 | 0.1 |
| Saccharin | 0.2 | 0.2 |
| Sodium monofluorphosphate | 1.12 | 1.12 |
| PEG 1500 | 5 | 5 |
| Sident 9 (abrasive silica) | 10 | 10 |
| Sident 22 S (thickening silica) | 8 | 8 |
| Sodium carboxymethylcellulose | 0.9 | 0.9 |
| Titanium (IV) oxide | 0.5 | 0.5 |
| Sodium lauryl sulfate (SLS) | 1.5 | 1.5 |
| Pellitorin solution (containing 10% Pellitorin) | — | 0.025 |
| Eucalyptus-menthol type flavoring from Example 37 | 1 | 1 |

### EXAMPLE 44

### Toothpaste (Calcium Carbonate Base)

| Component | A | B |
|---|---|---|
| Deionized water | 27.5 | To 100 |
| Saccharin | 0.2 | 0.2 |
| Solbrol M sodium salt | 0.2 | 0.2 |
| Sodium monofluorphosphate | 0.8 | 0.8 |
| Sorbitol 70% | 29 | 29 |
| Calcium carbonate | 35 | 35 |
| Sident 22 S (thickening silica) | 2.5 | 2.5 |
| Sodium carboxymethylcellulose | 1.3 | 1.3 |
| Titanium dioxide | 0.5 | 0.5 |
| Sodium lauryl sulfate | 2 | 2 |
| Pellitorin solution (containing 10% Pellitorin) | — | 0.02 |
| Eucalyptus-menthol type flavoring from Example 37 | 1 | 1 |

### EXAMPLE 45

### Toothpaste with Whitening Effect

| Component | A | B |
|---|---|---|
| Polyphosphate (Glass H, (n ≈ 21), Astaris) | 7 | 7 |
| Calcium peroxide | 1 | — |
| Na-percarbonate | — | 11 |
| Poloxamer 407 | 5 | 2 |
| Polyethylene glycol | 3 | — |
| Sorbitol, 70% in water | — | 22 |
| Glycerin | 43.8 | 12.5 |
| 1,2-propylene glycol | 4 | — |
| Na-Saccharin | 0.4 | 0.2 |
| Sodium bicarbonate | — | 5 |
| Sodium carbonate | 2 | 2 |
| Silica | 20 | 22 |
| Na-carboxymethylcellulose | 0.6 | 0.55 |
| Sodium lauryl sulfate | 1 | 4 |
| Xanthan gum | 0.2 | 0.2 |
| Titanium dioxide (Anatas) | 0.5 | 0.5 |
| Eucalyptus-menthol type flavoring from Example 37 | 1 | |
| Spicy-aromatic flavoring from Example 39 | | 1.25 |
| Distilled water | To 100 | To 100 |

### EXAMPLE 46

### Toothpastes with Tin and Zinc Salts

| Component | A | B | C |
|---|---|---|---|
| Sodium fluoride NaF | 0.42 | 0.5 | — |
| Tin fluoride SnF₂ | — | 0.9 | 0.95 |
| Tin chloride SnCl₂ | 1.5 | — | 2 |
| Zinc lactate | 2 | 2 | — |
| Zinc carbonate ZnCO₃ | — | 1 | 1.5 |
| Na-gluconate | — | 0.67 | 1.5 |
| Poloxamer 407 | 14.5 | — | — |
| Polyethylene glycol | 1 | 3 | — |
| Sorbitol, 70% in water | — | 38 | 37.5 |
| Glycerin | 37.5 | 5 | 14.4 |
| 1,2-propylene glycol | 7 | 5 | — |
| Na-Saccharin | 0.3 | 0.5 | 0.5 |
| Abrasive silica | 20 | 22.5 | 25 |
| Sodium hydroxide | — | 0.1 | 0.2 |
| Sodium lauryl sulfate | — | 2 | 1.5 |
| Na-polyphosphate | — | — | 4 |
| Tetrasodium pyrophosphate | 1 | 2.5 | — |
| Coloring (1% in water) | 0.4 | 0.5 | 0.5 |
| Eucalyptus-menthol type flavoring | 0.95 | — | — |
| from Example 37 | | | |
| Spicy-aromatic type flavoring | — | 1.2 | — |
| from Example 39 | | | |
| Wintergreen type flavoring | — | — | 1.15 |
| from Example 40 | | | |
| Distilled water | To 100 | To 100 | To 100 |

### EXAMPLE 47

### Phosphate-Based Toothpaste

| Component | Wt. % |
|---|---|
| Deionized water | 36.39 |
| Glycerin | 20 |
| Solbrol M (sodium salt) | 0.15 |
| Sodium monofluorphosphate | 0.76 |
| Saccharin | 0.2 |
| Dicalcium phosphate dihydrate | 36 |
| Aerosil ® 200 (Silica) | 3 |
| Sodium carboxymethylcellulose | 1.2 |
| Sodium lauryl sulfate (Texapon) | 1.3 |
| Spearmint type flavoring from Example 38 | 1 |

### EXAMPLE 48

### Toothpaste (Transparent Gel Formulation)

| Component | A | B |
|---|---|---|
| Sorbitol 70% | 63 | To 100 |
| Deionized water | 11.31 | 11.31 |
| Saccharin | 0.2 | 0.2 |
| Sodium monofluorphosphate | 1.14 | 1.14 |
| Solbrol | 0.15 | 0.15 |
| Trisodium phosphate | 0.1 | 0.1 |
| PEG 1500 (PEG 32) | 5 | 5 |
| Sident 9 (abrasive Silica) | 8 | 8 |
| Sident 22 S (thickening silica) | 8 | 8 |
| Sodium carboxymethylcellulose | 0.6 | 0.6 |
| Sodium lauryl sulfate | 1.5 | 1.5 |
| Pellitorin solution (containing 10% Pellitorin) | — | 0.025 |
| Spicy-aromatic type flavoring from Example 39 | 1 | — |
| Wintergreen type flavoring from Example 40 | — | 1 |

### EXAMPLE 49

### Mouthwash Concentrate with Wintergreen Flavor

| Component | Wt. % |
|---|---|
| Ethyl alcohol 96% | 42 |
| Cremophor RH 455 | 5 |
| Deionized water | 48.67 |
| Allantoin | 0.2 |
| Sodium saccharin 450 | 0.1 |
| Color L-Blue 5000 (1% in water) | 0.03 |
| Wintergreen type flavoring from Example 40 | 4 |

### EXAMPLE 50

### Mouthwash (Ready to Use, Alcohol-Free)

| Component | Wt. % |
|---|---|
| Cremophor RH 455 | 1.8 |
| Deionized water | 87.57 |
| Sorbitol 70% | 10 |
| Sodium fluoride | 0.18 |
| Sodium saccharin 450 | 0.1 |
| Solbrol M sodium salt | 0.15 |
| Mouthwash flavoring | 0.2 |
| from Example 42 | |

### EXAMPLE 51

### Mouthwash (Ready to Use, with Alcohol)

| Component | A | B | C |
|---|---|---|---|
| Ethyl alcohol 96% | 10 | 5 | 7 |
| Cremophor CO 40 | 1 | 1 | 1 |
| Benzoic acid | 0.1 | 0.12 | 0.1 |
| Deionized water | To 100 | To 100 | To 100 |
| Sorbitol 70% | 5 | 1 | 5 |
| Sodium saccharin 450 | 0.07 | 0.05 | 0.05 |
| L-Blue 5000 (1% in water) | 0.1 | 0.1 | 0.1 |
| Glycerin | — | 8 | — |
| 1,2-propylene glycol | — | 2 | 3 |
| Cetylpyridinium chloride | — | — | 0.07 |
| Hydrogen peroxide (35% H₂O₂ in water) | — | 3 | 4 |
| Wintergreen type flavoring | 0.25 | — | — |
| from Example 40 | | | |
| Mouthwash flavoring from Example 42 | — | 0.25 | 0.25 |

### EXAMPLE 52

### Tooth Cream and Mouthwash as a 2-in-1 Product

| Component | Wt. % |
|---|---|
| Ethanol, 96% | 5 |
| Sorbitol, 70% in water | 40 |
| Glycerin | 20 |
| Saccharin | 0.2 |
| Na-monofluorophosphate | 0.76 |
| Solbrol M, Na-salt | 0.15 |
| Abrasive silica (Sident 9) | 20 |
| Thickening silica (Sident 22S) | 2 |
| Na-carboxymethylcellulose | 0.3 |
| Sodium lauryl sulfate | 1.2 |
| Green coloring (1% in water) | 0.5 |
| Eucalyptus-menthol type flavoring | 1 |
| from Example 37 | |
| Distilled water | To 100 |

### EXAMPLE 53

### Standard Chewing Gum

| Component | A | B |
|---|---|---|
| Gum base | 21 | 21 |
| Glucose syrup | 17 | 17 |
| Glycerin | 0.5 | 0.5 |
| Powdered sugar | To 100 | To 100 |
| Peppermint type flavoring | 2 | — |
| from Example 41 | | |
| Spearmint type flavoring | — | 2 |
| from Example 38 | | |

### EXAMPLE 54

### Sugar-Free Chewing Gum

| Component | A | B |
|---|---|---|
| Gum base | 30 | 30 |
| Powdered Sorbitol | 40 | To 100 |
| Powdered Isomalt | 9.5 | 9.5 |
| Xylitol | 2 | 2 |
| Mannitol D | 3 | 3 |
| Aspartame | 0.1 | 0.1 |
| Acesulfame K | 0.1 | 0.1 |
| EmulgumTM (soya-lecithin with | 0.3 | 0.3 |
| a high content of phospholipids) | | |
| Sorbitol (70% in water) | 13 | 13 |
| 1,2-propylene glycol | — | 1 |
| Glycerin | 1 | — |
| Pellitorin solution (containing | — | 0.035 |
| 10% Pellitorin) | | |
| Peppermint type flavoring | 1 | 1 |
| from Example 41 | | |

### EXAMPLE 55

### Chewing Gum (with Sugar and Sugar-Free)

| Component | A | B |
|---|---|---|
| Gum Base | 21 | 30 |
| Glycerin | 0.5 | 1 |
| Glucose syrup | 16.5 | — |
| Powdered sugar | To 100 | — |
| Sorbitol (in powder form) | — | To 100 |
| Palatinit | — | 9.5 |
| Xylitol | — | 2 |
| Mannitol | — | 3 |
| Aspartame | — | 0.1 |
| Acesulfame K | — | 0.1 |
| Emulgum ™ (emulsifier) | — | 0.3 |
| Sorbitol 70%, in water | — | 14 |
| Spearmint type flavoring | 1 | 1.4 |
| from Example 38 | | |

### EXAMPLE 56

### Sugar-Free Chewing Gum

The gum base K1 consisted of 2.0% butyl rubber (isobutene-isoprene Copolymer, MW=400 000, 6.0% polyisobutene (MW=43,800), 43.5% polyvinyl acetate (MW=12,000), 31.5% polyvinyl acetate (MW=47,000), 6.75% triacetin and 10.25% calcium carbonate. The chewing gum base K1 and the chewing gum can be produced analogously to U.S. Pat. No. 5,601,858.

| Component | A | B | C |
|---|---|---|---|
| Chewing gum base K1 | 26 | 27 | 26 |
| Triacetin | 0.25 | 0.25 | 0.25 |
| Lecithin | 0.5 | 0.5 | 0.5 |
| Sorbitol, crystalline | To 100 | To 100 | To 100 |
| Mannitol | 15.3 | 15.2 | 15.1 |
| Glycerin | 12.1 | 12 | 11.8 |
| Saccharin-Na | 0.17 | — | 0.1 |
| Encapsulated aspartame | 1.08 | 1.18 | 1.08 |
| Amorphous silica | 1 | 1 | 1 |
| Cottonseed oil | 0.5 | 0.5 | 0.5 |
| Polyoxyethylene sorbitan monolaurate | 1 | 1 | 1 |
| (E-432) | | | |
| Encapsulated /-Carvone (loading: 30%) | — | 0.2 | — |
| /-menthyl-/-lactate | — | — | 0.2 |
| Spearmint type flavoring | 1 | — | 1.7 |
| from Example 38 | | | |
| Peppermint type flavoring | 0.5 | 1.4 | — |
| from Example 41 | | | |

### EXAMPLE 57

### Hardboiled Candy, Sugar-Free

| Component | A | B |
|---|---|---|
| Water | 2.24 | 2.24 |
| Isomalt | 94.98 | To 100 |
| Xylitol | 2.4 | 2.4 |
| Sucralose | 0.03 | 0.03 |
| Acesulfame K | 0.05 | 0.05 |
| Citric acid | 0.05 | 0.05 |
| Pellitorin solution (containing | — | 0.0075 |
| 10% Pellitorin) | | |
| Eucalyptus-menthol type flavoring | 0.25 | 0.2 |
| from Example 37 | | |

### EXAMPLE 58

### Hardboiled Candy

| Component | A | B |
|---|---|---|
| Water | 2.75 | 2.5 |
| Sugar | 60.1 | To 100 |
| Glucose syrup | 36.9 | 36 |
| Maltose | — | 2 |
| Palm nut oil | — | 0.8 |
| Citric acid | — | 0.25 |
| Ginseng extract | — | 0.4 |
| Blue coloring | — | 0.01 |
| Spearmint type flavoring | 0.25 | 0.35 |
| from Example 38 | | |

1. A fragrance and/or flavoring composition comprising one, two, three or a plurality of compounds of Formula (I), 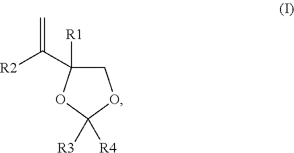
including the stereosiomers, wherein
R1 and R2 in each case and independently of one another is either hydrogen or methyl, preferably hydrogen, and
a) R3 and R4 form a ring with a total of 5, 6, 7 or 8 ring carbon atoms, wherein the ring optionally contains one or two double bonds and/or optionally 1 to a maximum of 3 oxygen atoms, and wherein the ring is optionally substituted by one or a plurality of branched or unbranched, bridged or unbridged, alkyl groups, alkenyl groups, cycloalkyl groups, cycloalkenyl groups, aryl groups, aryl alkyl groups, alkoxy alkyl groups or alkoxy aryl groups, and wherein R3 and R4 together comprise in total 3 through 30 carbon atoms,
or
b) R3 is either hydrogen or an organic radical with 1 through 15 carbon atoms, wherein the organic radical optionally contains 1 to a maximum of 3 oxygen atoms, preferably R3 is either hydrogen or a saturated or unsaturated, aromatic or aliphatic, branched or unbranched, cyclical or linear, bridged or unbridged structure element with 1 through 15 carbon atoms, wherein the structure element optionally contains 1 to a maximum of 3 oxygen atoms and the structure element is optionally substituted by 1 to a maximum of 3 alkyl- and/or alkenyl groups and/or by one or two hydroxy groups,
and
c) R4 is an organic radical with 2 through 15 carbon atoms, wherein the organic radical optionally contains 1 to a maximum of 3 oxygen atoms, preferably R4 is a saturated or unsaturated, aromatic or aliphatic, branched or unbranched, cyclical or linear, bridged or unbridged structure element with 2 through 15 carbon atoms, wherein the structure element optionally contains 1 to a maximum of 3 oxygen atoms and the structure element is optionally substituted by 1 to a maximum of 3 alkyl- and/or alkenyl groups and/or by one or two hydroxy groups,
and wherein at least one of the following conditions applies:
i. where R4 is a branched or unbranched alkyl radical with 4 through 15 carbon atoms with optionally one, two or a plurality of double bonds, at position 3 to position 4 of R4 a carbon-carbon single bond is present
or
ii. where R4 is a branched or unbranched alkyl radical with 2 through 15 carbon atoms with optionally one, two or a plurality of double bonds, R3 is not hydrogen,
and wherein the fragrance and/or flavoring composition contains at least 3, 4, 5, 6, 7, 8 or a plurality of further fragrance and/or flavoring substances, in which in each case a compound of Formula (I) is not involved,
and wherein the fragrance and/or flavoring composition does not contain 2-hexyl-4-vinyl-1,3-dioxolane.

2. The fragrance and/or flavoring composition as claimed in claim 1, wherein
R1 and R2 in each case and independently of one another is either hydrogen or methyl, in each case preferably hydrogen,
and
a) R3 and R4 together result in a cyclical alkyl system with 5, 6, 7 or 8 carbon atoms, which is optionally substituted by one or a plurality of branched or unbranched alkyl radicals selected from the group consisting of methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl, and wherein the alkyl system optionally contains one or two double bonds,
or
b) R3 is either hydrogen or a branched or unbranched alkyl radical with 1 through 15 carbon atoms and optionally one, two or a plurality of double bonds or a cyclical alkyl system with 5, 6 or 7 carbon atoms with optionally one, two or a plurality of double bonds, wherein the alkyl radical is preferably selected from the group consisting of methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl,
and
1. R4 is an aromatic compound, wherein the aromatic compound is optionally substituted by one, two or a plurality of alkyl radicals with 1 through 3 carbon atoms and/or one, two or a plurality of hydroxy radicals and/or one, two or a plurality of alkoxy radicals,
or
2. R4 is a mono-, bi- or tricyclic alkyl system, which is optionally substituted by one, two or a plurality of alkyl radicals, hydroxy radicals or alkoxy radicals, wherein the alkyl radicals are preferably selected from the group consisting of methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl,
or
3. R4 is a branched or unbranched alkyl radical with 2 through 15 carbon atoms with optionally one, two or a plurality of double bonds or a ring system with 5, 6 or 7 carbon atoms with optionally one, two or a plurality of double bonds.

3. The fragrance and/or flavoring composition as claimed in claim 1 or 2, wherein the fragrance and/or flavoring composition comprises one, two, three or a plurality of compounds of Formula (II): 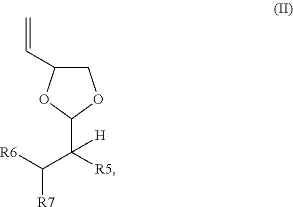
including the stereoisomers, wherein
a) R5 and R6 in each case and independently of one another is either hydrogen or methyl,
and b) R7 is an organic radical with 1 through 10 carbon atoms, wherein the organic radical optionally contains 1 to a maximum of 3 oxygen atoms, preferably R7 is a saturated or unsaturated, aromatic or aliphatic, branched or unbranched, cyclical or linear, bridged or unbridged structure element with 1 through 10 carbon atoms, wherein the structure element optionally contains 1 to a maximum of 3 oxygen atoms and the structure element is optionally substituted by 1 to a maximum of 3 alkyl- and/or alkenyl groups and/or by a hydroxy group,
and
wherein where R7 is a branched or unbranched alkyl radical with 1 through 10 carbon atoms, at position 1 to position 2 of R7 a carbon-carbon single bond is present.

4. The fragrance and/or flavoring composition as claimed in claim 3, wherein the fragrance and/or flavoring composition comprises one, two, three or a plurality of compounds of Formula (II), wherein
a) R5 and R6 in each case and independently of one another is either hydrogen or methyl,
and
b) R7 is a branched or unbranched alkyl radical with 1 through 10 carbon atoms, preferably selected from the group consisting of methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl,
or
R7 is a cyloalkyl radical with 5, 6 or 7 carbon atoms, that is optionally substituted by one or a plurality of alkyl radicals, selected from the group consisting of methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl, a hydroxy radical or an alkoxy radical.

5. The fragrance and/or flavoring composition as claimed in any one of the preceding claims, wherein the fragrance and/or flavoring composition comprises one, two, three or a plurality of compounds, selected from the group consisting of:
2-isobutyl-4-vinyl-[1,3]dioxolane,
2-secbutyl-4-vinyl-[1,3]dioxolane,
2-isopropyl-4-vinyl-[1,3]dioxolane,
2-(3,5-dimethylcyclohex-3-enyl)-4-vinyl-[1,3]dioxolane,
2-(2,4-dimethylcyclohex-3-enyl)-4-vinyl-[1,3]dioxolane,
2-(2,6-dimethylhept-5-enyl)-4-vinyl-[1,3]dioxolane,
2-ethyl-2-(2-methylbutyl)-4-vinyl-[1,3]dioxolane,
2-(2,4,4-trimethylpentyl)-4-vinyl-[1,3]dioxolane,
2-(3,5-dimethylhex-4-enyl)-2-methyl-4-vinyl-[1,3]dioxolane,
2-methyl-2-(4-methylpent-3-enyl)-4-vinyl-[1,3]dioxolane,
8-tertbutyl-2-vinyl-1,4-dioxaspiro-[4.5]-decane,
2-(2,6-dimethylhepta-1,5-dienyl-4-vinyl-[1,3]dioxolane,
2-[2-(4-methylcyclohex-3-enyl)-propyl]-4-vinyl-[1,3]dioxolane,
2-phenyl-4-vinyl-[1,3]dioxolane,
2-cyclohexyl-4-vinyl-[1,3]dioxolane,
2-(2,2,3-trimethylcyclopent-3-enylmethyl)-4-vinyl-[1,3]dioxolane,
2-vinyl-1,4-dioxaspiro[4.5]decane,
7-methyl-2-vinyl-1,4-dioxaspiro[4.5]decane,
2-(2-methylpropenyl)-4-vinyl-[1,3]dioxolane, and
2-methyl-2-(3-methylbutyl)-4-vinyl-[1,3]dioxolane.

6. The fragrance and/or flavoring composition as claimed in any one of the preceding claims, wherein the fragrance and/or flavoring composition comprises a total quantity of compounds of Formula (I) and/or (II) of 0.0001 through 70 Wt. %, preferably 0.001 through 50 Wt. % and particularly preferably 0.01 through 20 Wt. %, in each case in relation to the total quantity of fragrance and/or flavoring substances, wherein the fragrance or flavoring composition preferably comprises one, two, three, four, five or a plurality of fragrance and/or flavoring substances, that impart a flowery and/or fruity odor or taste.

7. A compound of Formula (I) 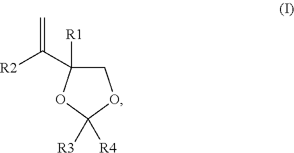
including the stereoisomers,
wherein
R1 and R2 in each case and independently of one another is either hydrogen or methyl, preferably hydrogen
and
a) R3 and R4 together result in a cyclical alkyl system with 5, 6, 7 or 8 carbon atoms, which is optionally substituted by one or a plurality of branched or unbranched alkyl radicals selected from the group consisting of methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl, and wherein the alkyl system optionally contains one or two double bonds,
or
b) R3 is either hydrogen or a branched or unbranched alkyl radical with 1 through 15 carbon atoms and optionally one, two or a plurality of double bonds or a cyclical alkyl system with 5, 6 or 7 carbon atoms with optionally one, two or a plurality of double bonds, wherein the alkyl radical is preferably selected from the group consisting of methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl,
and
1. R4 is an aromatic compound, wherein the aromatic compound optionally is substituted by one, two or a plurality of alkyl radicals with 1 through 3 carbon atoms and/or one, two or a plurality of hydroxy radicals and/or one, two or a plurality of alkoxy radicals,
or
2. R4 is a mono-, bi- or tricyclic alkyl system, which optionally is substituted by one, two or a plurality of alkyl radicals, hydroxy radicals or alkoxy radicals, wherein the alkyl radicals are preferably selected from the group consisting of methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl,
or
3. R4 is a branched or unbranched alkyl radical with 2 through 15 carbon atoms with optionally one, two or a plurality of double bonds or a ring system with 5, 6 or 7 carbon atoms with optionally one, two or a plurality of double bonds,
and wherein at least one of the following conditions applies:
i. where R4 is a branched or unbranched alkyl radical with 4 through 15 carbon atoms with optionally one, two or a plurality of double bonds, at position 3 to position 4 of R4 a carbon-carbon single bond is present
or
ii where R4 is a branched or unbranched alkyl radical with 2 through 15 carbon atoms with optionally one, two or a plurality of double bonds, R3 is a branched or unbranched alkyl radical with 1 through 15 carbon atoms and optionally one, two or a plurality of double bonds,
excluding the following compounds:
2-ethyl-4-vinyl-[1,3]dioxolane2-propyl-4-vinyl-[1,3]dioxolane
2-isopropyl-4-vinyl-[1,3]dioxolane
2-butyl-4-vinyl-[1,3]dioxolane
2-isobutyl-4-vinyl-[1,3]dioxolane
2-pentyl-4-vinyl-[1,3]dioxolane
2-hexyl-4-vinyl-[1,3]dioxolane
2-heptyl-4-vinyl-[1,3]dioxolane
2-cyclohexyl-4-vinyl-[1,3]dioxolane
2-phenyl-4-vinyl-[1,3]dioxolane
2-o-tolyl-4-vinyl-[1,3]dioxolane
2-m-tolyl-4-vinyl-[1,3]dioxolane
2-p-tolyl-4-vinyl-[1,3]dioxolane
2-(2-methoxy-phenyl)-4-vinyl-[1,3]dioxolane
2-(3-methoxy-phenyl)-4-vinyl-[1,3]dioxolane
2-(4-methoxy-phenyl)-4-vinyl-[1,3]dioxolane
5-(4-vinyl-[1,3]dioxolane-2-yl)-benzo[1,3]dioxol
2-furan-3-yl-4-vinyl-[1,3]dioxolane
2-ethyl-2-methyl-4-vinyl-[1,3]dioxolane
2-methyl-2-propyl-4-vinyl-[1,3]dioxolane
2-butyl-2-methyl-4-vinyl-[1,3]dioxolane
2-isopropyl-2-methyl-4-vinyl-[1,3]dioxolane
2-methyl-2-(3-methylbutyl)-4-vinyl-[1,3]dioxolane
2-methyl-2-(4-methyl-pentyl)-4-vinyl-[1,3]dioxolane
2,2-diethyl-4-vinyl-[1,3]dioxolane
2,2-dipropyl-4-vinyl-[1,3]dioxolane
2,2-diisopropyl-4-vinyl-[1,3]dioxolane
2-methyl-2-pentyl-4-vinyl-[1,3]dioxolane
2-hexyl-2-methyl-4-vinyl-[1,3]dioxolane
2-methyl-2-octyl-4-vinyl-[1,3]dioxolane
2-methyl-2-nonyl-4-vinyl-[1,3]dioxolane
2-methyl-2-phenyl-4-vinyl-[1,3]dioxolane
2-cyclohexyl-2-methyl-4-vinyl-[1,3]dioxolane
2-cyclopentyl-4-vinyl-[1,3]dioxolane
2-cycloheptyl-4-vinyl-[1,3]dioxolane
2,2-pentamethylen-4-vinyl-[1,3]-dioxolane
2-methyl-2-isopropyl-4-vinyl-[1,3]-dioxolane
2,2-hexamethyl en-4-vinyl-[1,3]-dioxolane
2-but-3-enyl-2-methyl-4-vinyl-[1,3]dioxolane
(S)-2,2-diethyl-4-vinyl-[1,3]dioxolane
(R)-2-vinyl-1,4-dioxa-spiro[4.5]decane
(S)-2-vinyl-1,4-dioxa-spiro[4.5]decane
2-((E)-propenyl)-4-vinyl-[1,3]dioxolane
2-(4-vinyl-[1,3]dioxolane-2-yl)-phenol
2,4-divinyl-[1,3]dioxolane
2-tert-butyl-2-methyl-4-vinyl-[1,3]dioxolane
2-butyl-2-ethyl-4-vinyl-[1,3]dioxolane
2-(2-methyl-cyclohexyl)-4-vinyl-[1,3]dioxolane
2-(4-methyl-cyclohexyl)-4-vinyl-[1,3]dioxolane
(S)-2-methyl-2-vinyl-1,4-dioxa-spiro[4.5]decane
2-methyl-2-vinyl-1,4-dioxa-spiro[4.5]decane
(R)-2-(4-methoxy-phenyl)-4-methyl-4-vinyl-[1,3]dioxolane
4-isopropenyl-2-p-tolyl-[1,3]dioxolane
4-isopropenyl-2-phenyl-[1,3]dioxolane
(2S,4S)-4-methyl-2-((R)-1-methyl-allyl)-4-vinyl-[1,3]dioxolane
(2R,4R)-4-methyl-2-((R)-1-methyl-allyl)-4-vinyl-[1,3]dioxolane
(2S,4R)-4-methyl-2-((R)-1-methyl-allyl)-4-vinyl-[1,3]dioxolane
(2R,4S)-4-methyl-2-((R)-1-methyl-allyl)-4-vinyl-[1,3]dioxolane
2-ethyl-2,4-dimethyl-4-vinyl-[1,3]dioxolane
4-methyl-2-(1-phenyl-ethyl)-4-vinyl-[1,3]dioxolane
2-isobutyl-2-methyl-4-vinyl-[1,3]dioxolane.

8. A use of a compound of Formula (I) 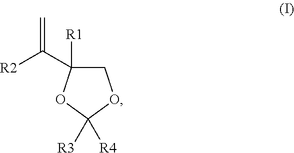
including the stereoisomers,
wherein
R1 and R2 in each case and independently of one another is either hydrogen or methyl, preferably hydrogen, and
a) R3 and R4 form a ring with a total of 5, 6, 7 or 8 ring carbon atoms, wherein the ring optionally contains one or two double bonds and/or optionally 1 to a maximum of 3 oxygen atoms, and wherein the ring is optionally substituted by one or a plurality of branched or unbranched, bridged or unbridged, alkyl groups, alkenyl groups, cycloalkyl groups, cycloalkenyl groups, aryl groups, aryl alkyl groups, alkoxy alkyl groups or alkoxy aryl groups, and wherein R3 and R4 together comprise in total 3 through 30 carbon atoms,
or
b) R3 is either hydrogen or an organic radical with 1 through 15 carbon atoms, wherein the organic radical optionally contains 1 to a maximum of 3 oxygen atoms, preferably R3 is either hydrogen or a saturated or unsaturated, aromatic or aliphatic, branched or unbranched, cyclical or linear, bridged or unbridged structure element with 1 through 15 carbon atoms, wherein the structure element optionally contains 1 to a maximum of 3 oxygen atoms and the structure element is optionally substituted by 1 to a maximum of 3 alkyl- and/or alkenyl groups and/or by one or two hydroxy groups,
and
R4 is an organic radical with 2 through 15 carbon atoms, wherein the organic radical optionally contains 1 to a maximum of 3 oxygen atoms, preferably R4 is a saturated or unsaturated, aromatic or aliphatic, branched or unbranched, cyclical or linear, bridged or unbridged structure element with 2 through 15 carbon atoms, wherein the structure element optionally contains 1 to a maximum of 3 oxygen atoms and the structure element is optionally substituted by 1 to a maximum of 3 alkyl- and/or alkenyl groups and/or by one or two hydroxy groups
and wherein at least one of the following conditions applies:
i. where R4 is a branched or unbranched alkyl radical with 4 through 15 carbon atoms with optionally one, two or a plurality of double bonds, at position 3 to position 4 of R4 a carbon-carbon single bond is present
or
ii. where R4 is a branched or unbranched alkyl radical with 2 through 15 carbon atoms with optionally one, two or a plurality of double bonds, R3 is not hydrogen,
as a fragrance and/or flavoring substance for imparting, enhancing and/or modifying one, two or a plurality of scents and/or flavors, wherein no fragrance and/or flavor of pineapple is imparted.

9. The use as claimed in claim 8, wherein
R1 and R2 in each case and independently of one another is either hydrogen or methyl, preferably hydrogen
and
a) R3 and R4 together result in a cyclical alkyl system with 5, 6, 7 or 8 carbon atoms, which is optionally substituted by one or a plurality of branched or unbranched alkyl radicals selected from the group consisting of methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl, and wherein the alkyl system optionally contains one or two double bonds,
or
b) R3 is either hydrogen or a branched or unbranched alkyl radical with 1 through 15 carbon atoms and optionally one, two or a plurality of double bonds or a cyclical alkyl system with 5, 6 or 7 carbon atoms with optionally one, two or a plurality of double bonds, wherein the alkyl radical is preferably selected from the group consisting of methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl,
and
1. R4 is an aromatic compound, wherein the aromatic compound optionally is substituted by one, two or a plurality of alkyl radicals with 1 through 3 carbon atoms and/or one, two or a plurality of hydroxy radicals and/or one, two or a plurality of alkoxy radicals,
or
2. R4 is a mono-, bi- or tricyclic alkyl system, which optionally is substituted by one, two or a plurality of alkyl radicals, hydroxy radicals or alkoxy radicals, wherein the alkyl radicals are preferably selected from the group consisting of methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl,
or
3. R4 is a branched or unbranched alkyl radical with 2 through 15 carbon atoms with one, two or a plurality of double bonds or a ring system with 5, 6 or 7 carbon atoms with one, two or a plurality of double bonds,
and wherein at least one of the following conditions applies:
i. where R4 is a branched or unbranched alkyl radical with 4 through 15 carbon atoms with optionally one, two or a plurality of double bonds, at position 3 to position 4 of R4 a carbon-carbon single bond is present
or
ii. where R4 is a branched or unbranched alkyl radical with 2 through 15 carbon atoms with optionally one, two or a plurality of double bonds, R3 is a branched or unbranched alkyl radical with 1 through 15 carbon atoms and optionally one, two or a plurality of double bonds,
preferably a compound according to Formula (II) 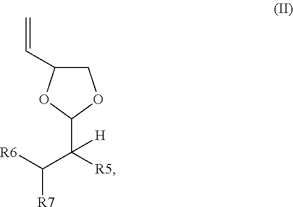
including the stereoisomers,
wherein
a) R5 and R6 in each case and independently of one another is either hydrogen or methyl,
and
b) R7 is a branched or unbranched alkyl radical with 1 through 10 carbon atoms, preferably selected from the group consisting of methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl,
or
R7 is a cyloalkyl radical with 5, 6 or 7 carbon atoms, which is optionally substituted by one or a plurality of alkyl radicals, selected from the group consisting of methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl, a hydroxy radical or an alkoxy radical,
wherein where R7 is a branched or unbranched alkyl radical with 1 through 10 carbon atoms, at position 1 to position 2 of R7 a carbon-carbon single bond is present.

10. The use as claimed in claim 8 or 9, wherein the compound is selected from the group consisting of:
2-isobutyl-4-vinyl-[1,3]dioxolane,
2-secbutyl-4-vinyl-[1,3]dioxolane,
2-isopropyl-4-vinyl-[1,3]dioxolane,
2-(3,5-dimethylcyclohex-3-enyl)-4-vinyl-[1,3]dioxolane,
2-(2,4-dimethylcyclohex-3-enyl)-4-vinyl-[1,3]dioxolane,
2-(2,6-dimethylhept-5-enyl)-4-vinyl-[1,3]dioxolane,
2-ethyl-2-(2-methylbutyl)-4-vinyl-[1,3]dioxolane,
2-(2,4,4-trimethylpentyl)-4-vinyl-[1,3]dioxolane,
2-(3,5-dimethylhex-4-enyl)-2-methyl-4-vinyl-[1,3]dioxolane,
2-methyl-2-(4-methylpent-3-enyl)-4-vinyl-[1,3]dioxolane,
8-tertbutyl-2-vinyl-1,4-dioxaspiro-[4.5]-decane,
2-(2,6-dimethylhepta-1,5-dienyl-4-vinyl-[1,3]dioxolane,
2-[2-(4-methylcyclohex-3-enyl)-propyl]-4-vinyl-[1,3]dioxolane,
2-phenyl-4-vinyl-[1,3]dioxolane,
2-cyclohexyl-4-vinyl-[1,3]dioxolane,
2-(2,2,3-trimethylcyclopent-3-enylmethyl)-4-vinyl-[1,3]dioxolane,
2-vinyl-1,4-dioxaspiro[4.5]decane,
7-methyl-2-vinyl-1,4-dioxaspiro[4.5]decane,
2-(2-methylpropenyl)-4-vinyl-[1,3]dioxolane, and
2-methyl-2-(3-methylbutyl)-4-vinyl-[1,3]dioxolane.

11. The use as claimed in any one of claims 8 through 10 for imparting, enhancing and/or modifying one, two, three or a plurality of the scents and/or flavors flowery, fatty, green olives, leathery, fruity, green, flowers, rose, myrrh, pear, mango, grapefruit, melon, lemon, linalool, Mirabelle, spearmint, phenolic, mushroomy, metallic, chamomile, apple, carbinol, aromatic, watery, fresh, herby.

12. The perfumed and/or flavored item, comprising a fragrance and/or flavoring composition as claimed in any one of claims 1 through 5 and/or a compound as claimed in claim 6 or 7,
wherein the perfumed item is preferably selected from the group consisting of detergents, hygiene or care products, in particular in the range of body and hair care, cosmetic and household, and the flavored items, is preferably selected from the group consisting of foodstuffs, semi-luxury foods, drinks, oral care products or pharmaceutical products.

13. A method for producing a compound of Formula (I) as defined in claims 1 through 5, comprising the following step:
reaction of a compound of Formula (III) with a compound of Formula (IV) to form the compound of Formula (I), preferably in the presence of copper(I)chloride, according to the scheme 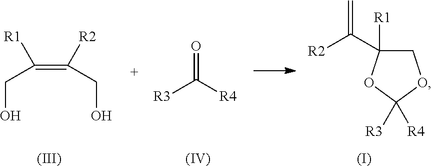
wherein the radicals R1, R2, R3 and R4 of Formulas (III) and (IV) are defined in the same way as the corresponding radicals for Formula (I).

14. The method as claimed in claim 13, wherein the compound of Formula (I) is selected from the group consisting of:
2-isobutyl-4-vinyl-[1,3]dioxolane,
2-secbutyl-4-vinyl-[1,3]dioxolane,
2-isopropyl-4-vinyl-[1,3]dioxolane,
2-(3,5-dimethylcyclohex-3-enyl)-4-vinyl-[1,3]dioxolane,
2-(2,4-dimethylcyclohex-3-enyl)-4-vinyl-[1,3]dioxolane,
2-(2,6-dimethylhept-5-enyl)-4-vinyl-[1,3]dioxolane,
2-ethyl-2-(2-methylbutyl)-4-vinyl-[1,3]dioxolane,
2-(2,4,4-trimethylpentyl)-4-vinyl-[1,3]dioxolane,
2-(3,5-dimethylhex-4-enyl)-2-methyl-4-vinyl-[1,3]dioxolane,
2-methyl-2-(4-methylpent-3-enyl)-4-vinyl-[1,3]dioxolane,
8-tertbutyl-2-vinyl-1,4-dioxaspiro-[4.5]-decane,
2-(2,6-dimethylhepta-1,5-dienyl-4-vinyl-[1,3]dioxolane,
2-[2-(4-methylcyclohex-3-enyl)-propyl]-4-vinyl-[1,3]dioxolane,
2-phenyl-4-vinyl-[1,3]dioxolane,
2-cyclohexyl-4-vinyl-[1,3]dioxolane,
2-(2,2,3-trimethylcyclopent-3-enylmethyl)-4-vinyl-[1,3]dioxolane,
2-vinyl-1,4-dioxaspiro[4.5]decane,
7-methyl-2-vinyl-1,4-dioxaspiro[4.5]decane,
2-(2-methylpropenyl)-4-vinyl-[1,3]dioxolane, and
2-methyl-2-(3-methylbutyl)-4-vinyl-[1,3]dioxolane.

15. A method for imparting, modifying and/or enhancing an odor and/or taste without imparting the odor or taste of the pineapple note, wherein a sensorially effective quantity
of a fragrance and/or flavoring composition as claimed in any one of claims 1 through 6
or
a compound as claimed in claim 7 is brought into contact or mixed with a product, preferably for imparting, modifying and/or enhancing one, two or a plurality of the scents and/or flavors flowery, fatty, green olives, leathery, fruity, green, flowers, rose, myrrh, pear, mango, grapefruit, melon, lemon, linalool, Mirabelle, spearmint, phenolic, mushroomy, metallic, camomile, apple, carbinol, aromatic, watery, fresh, herby.
